(19) United States
(12) Patent Application Publication
(10) Pub. No.: US 2019/0198770 A1
(43) Pub. Date: Jun. 27, 2019

(54) HETEROCYCLIC COMPOUND, COMPOSITION INCLUDING HETEROCYCLIC COMPOUND, AND ORGANIC LIGHT-EMITTING DEVICE INCLUDING HETEROCYCLIC COMPOUND

(71) Applicant: Samsung Electronics Co., Ltd., Suwon-si (KR)

(72) Inventors: Keisuke KORAI, Kanagawa (JP); Rie SAKURAI, Suwon-si (KR); Mitsunori ITO, Kanagawa (JP); Tadao YAGI, Hwaseong-si (KR)

(21) Appl. No.: 16/233,751

(22) Filed: Dec. 27, 2018

(30) Foreign Application Priority Data
Dec. 27, 2017 (JP) 2017-252570
Nov. 14, 2018 (KR) 10-2018-0140095

(51) Int. Cl. H01L 51/00; C07D 403/10; C09K 11/06

(52) U.S. Cl. CPC H01L 51/0067; C07D 403/10; H01L 51/0072; H01L 51/5016; H01L 51/0035; H01L 51/0007; C09K 11/06

(57) ABSTRACT

A heterocyclic compound represented by Formula 1:

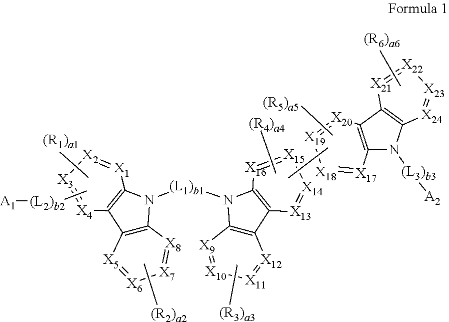

wherein, in Formula 1, groups and variables are the same as described in the specification.

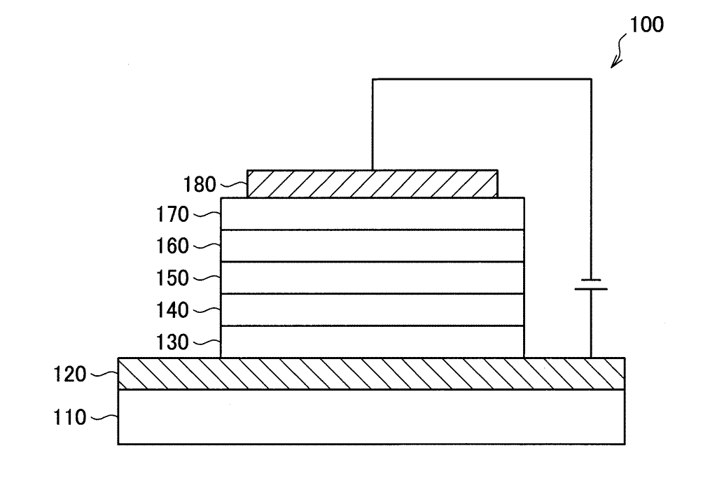

### CROSS-REFERENCE TO RELATED APPLICATIONS

This application claims priority to Japanese Patent Application No. 2017-252570, filed on Dec. 27, 2017, in the Japanese Patent Office, and Korean Patent Application No. 10-2018-0140095, filed on Nov. 14, 2018, in the Korean Intellectual Property Office, and all the benefits accruing therefrom under 35 U.S.C. § 119, the contents of which are incorporated herein in their entireties by reference.

### BACKGROUND

### 1. Field

The present disclosure relates to a heterocyclic compound, a material for an organic light-emitting device including the heterocyclic compound, and an organic light-emitting device including the heterocyclic compound.

### 2. Description of the Related Art

Organic light-emitting devices (OLEDs) are self-emission devices that have relatively wide viewing angles, relatively high contrast ratios, relatively short response times, and increased luminance, driving voltage, and response speed characteristics. OLEDs may produce full-color images.

Typical OLEDs include an anode, a cathode, and an organic layer that is between the anode and the cathode and includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. Carriers, such as holes and electrons, recombine in the emission layer to produce excitons. These excitons transit from an excited state to a ground state to thereby generate light.

Various types of organic light emitting devices are known. However, there still remains a need in OLEDs having low driving voltage, high efficiency, high brightness, and long lifespan.

### SUMMARY

Provided are a heterocyclic compound, a composition including the heterocyclic compound, and an organic light-emitting device including the heterocyclic compound, for example, the organic light-emitting device may have excellent current efficiency and emission lifespan by including the heterocyclic compound. Also, the heterocyclic compound may have suitable properties in using solution coating.

Additional aspects will be set forth in part in the description which follows and, in part, will be apparent from the description, or may be learned by practice of the presented embodiments.

According to an aspect of an embodiment, a heterocyclic compound may be represented by Formula 1:

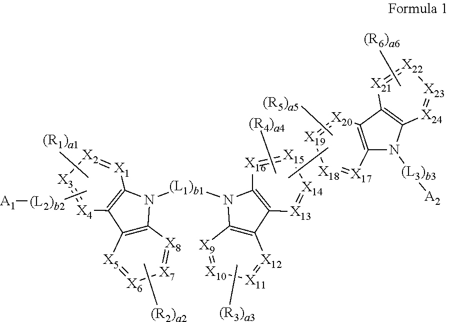

wherein, in Formula 1,

X₁ to X₂₄ may each independently be selected from carbon (C) and nitrogen (N),

at least one selected from X₁ and X₄ may be C and be bound to -(L₂)_{b2}-A₁,

L₁ may be selected from a substituted or unsubstituted C₁-C₂₀ alkylene group, a substituted or unsubstituted C₃-C₂₀ cycloalkylene group, a substituted or unsubstituted C₅-C₆₀ carbocyclic group, and a substituted or unsubstituted C₁-C₆₀ heterocyclic group,

L₂ and L₃ may each independently be selected from a single bond, a substituted or unsubstituted C₁-C₂₀ alkylene group, a substituted or unsubstituted C₃-C₂₀ cycloalkylene group, a substituted or unsubstituted C₅-C₆₀ carbocyclic group, and a substituted or unsubstituted C₁-C₆₀ heterocyclic group,

b1 may be an integer selected from 1 to 5,

b2 and b3 may each independently be an integer from 0 to 5,

A₁ and A₂ may each independently be selected from a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, and a substituted or unsubstituted C₅-C₆₀ heteroaryl group, provided that at least one selected from A₁ and A₂ may be a C₅-C₆₀ heteroaryl group,

R₁ to R₆ may each independently be selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, and a monovalent non-aromatic condensed heteropolycyclic group,

a1, a4, and a5 may each independently be an integer from 0 to 3, and

a2, a3, and a6 may each independently be an integer from 0 to 4.

According to an aspect of another embodiment, a composition may include at least one heterocyclic compound represented by Formula 1.

According to an aspect of still another embodiment, an organic light-emitting device may include:

a first electrode;

a second electrode; and

an organic layer disposed between the first electrode and the second electrode,

wherein the organic layer includes an emission layer and at least one heterocyclic compound.

### BRIEF DESCRIPTION OF THE DRAWINGS

These and/or other aspects will become apparent and more readily appreciated from the following description of the embodiments, taken in conjunction with the FIGURE, which is a schematic cross-sectional view illustrating an organic light-emitting device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

One or more embodiments of the inventive concept of the present disclosure will now be described more fully with reference to the accompanying drawings. In addition, in the present specification and drawings, like reference numerals in the drawings denote like elements, and thus, their description will be omitted. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within±30%, 20%, 10%, 5% of the stated value.

In an embodiment, a heterocyclic compound is provided. The heterocyclic compound may be represented by Formula 1:

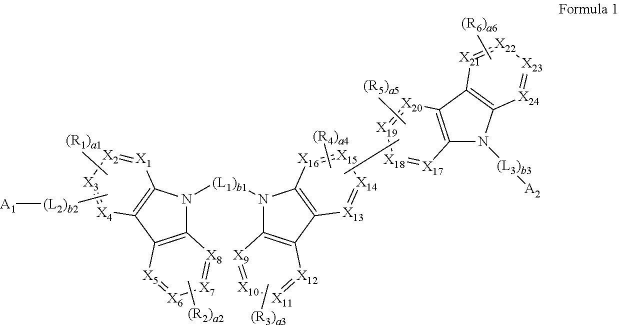

wherein, in Formula 1, X₁ to X₂₄ may each independently be C or N, and at least one selected from X₁ and X₄ may be C and be bound to -(L₂)_{b2}-A₁.

For example, X₁ may be C and be bound to -(L₂)_{b2}-A₁. For example, X₄ may be C and be bound to -(L₂)_{b2}-A₁.

In some embodiments, at least one selected from X₁ to X₈ may be N.

In some embodiments, X₉ to X₂₄ may each be C.

In some embodiments, X₁ to X₂₄ may each be C.

In Formula 1, L₁ may be selected from a substituted or unsubstituted C₁-C₂₀ alkylene group, a substituted or unsubstituted C₃-C₂₀ cycloalkylene group, a substituted or unsubstituted C₅-C₆₀ carbocyclic group, and a substituted or unsubstituted C₁-C₆₀ heterocyclic group.

In some embodiments, L₁ may be selected from a phenylene group, a biphenylene group, a terphenylene group, a tetraphenylene group, an indenylene group, a naphthylene group, an acenaphthylene group, a fluorenylene group, a spiro-bifluorenylene group, a phenanthrenyl group, an anthracenylene group, a fluoranthrenylene group, a triphenylenylene group, a pyrenylene group, a chrysenylene group, a pyrrolylene group, an imidazolylene group, a pyrazolylene group, a triazolylene group, a pyridinylene group, a pyridazinylene group, a pyrimidinylene group, a pyridazinylene group, a triazinylene group, a furanylene group, a thienylene group, an oxazolylene group, an isoxazolylene group, a thiazolylene group, an isothiazolylene group, an oxadiazolylene group, an isoxadiazolylene group, a thiadiazolylene group, an iso-thiadiazolylene group, a pyranylene group, an imidazolylene group, a purinylene group, a quinolinylene group, an isoquinolinylene group, a benzoquinolinylene group, a phthalazinylene group, a naphthyridinylene group, a quinazolinylene group, a cinnolinylene group, a phenanthridinylene group, an acridinylene group, a phenanthrolinylene group, a phenazinylene group, a benzoxazinylene group, a benzothiazolylene group, benzimidazolylene group, an isoindolinylene group, an indolinylene group, a benzofuranylene group, a benzothiophenylene group, a carbazolylene group, an azacarbazolylene group, a dibenzofuranylene group, an azadibenzofuranylene group, a dibenzothiophenylene group, an azadibenzothiophenylene group, a benzocarbazolylene group, a naphthobenzocarbazolylene group, a naphthobenzofuranylene group, a naphthobenzothiophenylene group, an imidazopyrimidinylene group, an imidazopyridinylene group, a methylene group, an ethylene group, a propylene group, an iso-propylene group, a butylene group, a pentylene group, a hexylene group, a cyclobutylene group, a cyclopentylene group, and a cyclohexylene group; and

a phenylene group, a biphenylene group, a terphenylene group, a tetraphenylene group, an indenylene group, a naphthylene group, an acenaphthylene group, a fluorenylene group, a spiro-bifluorenylene group, a phenanthrenyl group, an anthracenylene group, a fluoranthrenylene group, a triphenylenylene group, a pyrenylene group, a chrysenylene group, a pyrrolylene group, an imidazolylene group, a pyrazolylene group, a triazolylene group, a pyridinylene group, a pyridazinylene group, a pyrimidinylene group, a pyridazinylene group, a triazinylene group, a furanylene group, a thienylene group, an oxazolylene group, an isoxazolylene group, a thiazolylene group, an isothiazolylene group, an oxadiazolylene group, an isoxadiazolylene group, a thiadiazolylene group, an iso-thiadiazolylene group, a pyranylene group, an imidazolylene group, a purinylene group, a quinolinylene group, an isoquinolinylene group, a benzoquinolinylene group, a phthalazinylene group, a naphthyridinylene group, a quinazolinylene group, a cinnolinylene group, a phenanthridinylene group, an acridinylene group, a phenanthrolinylene group, a phenazinylene group, a benzoxazinylene group, a benzothiazolylene group, benzimidazolylene group, an isoindolinylene group, an indolinylene group, a benzofuranylene group, a benzothiophenylene group, a carbazolylene group, an azacarbazolylene group, a dibenzofuranylene group, an azadibenzofuranylene group, a dibenzothiophenylene group, an azadibenzothiophenylene group, a benzocarbazolylene group, a naphthobenzocarbazolylene group, a naphthobenzofuranylene group, a naphthobenzothiophenylene group, an imidazopyrimidinylene group, an imidazopyridinylene group, a methylene group, an ethylene group, a propylene group, an iso-propylene group, a butylene group, a pentylene group, a hexylene group, a cyclobutylene group, a cyclopentylene group, and a cyclohexylene group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

For example, L₁ may be selected from a phenylene group, a fluorenylene group, a 9,9-diarylfluorenylene group, a 9,9-dialkylfluorenylene group, a spirobifluorenylene group, and a triphenylenylene group. In some embodiments, when L₁ is a phenylene group, L₁ may be an m-phenylene group or a p-phenylene group.

In some embodiments, L₁ may be a substituted or unsubstituted heterocyclic group containing 5 to 60 ring-forming atoms. When L₁ is a substituted or unsubstituted heterocyclic group containing 5 to 60 ring-forming atoms, L₁ may be a substituted or unsubstituted condensed cyclic group including O or S as a heteroatom and three or more condensed rings, for example, a substituted or unsubstituted dibenzofuranylene group or a substituted or unsubstituted dibenzothiophenylene group.

In Formula 1, L₂ and L₃ may each independently be selected from a single bond, a substituted or unsubstituted alkylene group, a substituted or unsubstituted C₃-C₂₀ cycloalkylene group, a substituted or unsubstituted C₅-C₆₀ carbocyclic group, and a substituted or unsubstituted C₁-C₆₀ heterocyclic group.

In some embodiments, in Formula 1, L₂ and L₃ may each independently be selected from a single bond, a substituted or unsubstituted alkylene group, a substituted or unsubstituted C₃-C₂₀ cycloalkylene group, a substituted or unsubstituted C₅-C₆₀ carbocyclic group, and a substituted or unsubstituted C₁-C₆₀ heterocyclic group.

In some embodiments, L₂ and L₃ may each independently be selected from a single bond, a phenylene group, a biphenylene group, a terphenylene group, a tetraphenylene group, an indenylene group, a naphthylene group, an acenaphthylene group, a fluorenylene group, a spiro-bifluorenylene group, a phenanthrenyl group, an anthracenylene group, a fluoranthrenylene group, a triphenylenylene group, a pyrenylene group, a chrysenylene group, a pyrrolylene group, an imidazolylene group, a pyrazolylene group, a triazolylene group, a pyridinylene group, a pyridazinylene group, a pyrimidinylene group, a pyridazinylene group, a triazinylene group, a furanylene group, a thienylene group, an oxazolylene group, an isoxazolylene group, a thiazolylene group, an isothiazolylene group, an oxadiazolylene group, an isoxadiazolylene group, a thiadiazolylene group, an iso-thiadiazolylene group, a pyranylene group, an imidazolylene group, a purinylene group, a quinolinylene group, an isoquinolinylene group, a benzoquinolinylene group, a phthalazinylene group, a naphthyridinylene group, a quinazolinylene group, a cinnolinylene group, a phenanthridinylene group, an acridinylene group, a phenanthrolinylene group, a phenazinylene group, a benzoxazinylene group, a benzothiazolylene group, benzimidazolylene group, an isoindolinylene group, an indolinylene group, a benzofuranylene group, a benzothiophenylene group, a carbazolylene group, an azacarbazolylene group, a dibenzofuranylene group, an azadibenzofuranylene group, a dibenzothiophenylene group, an azadibenzothiophenylene group, a benzocarbazolylene group, a naphthobenzocarbazolylene group, a naphthobenzofuranylene group, a naphthobenzothiophenylene group, an imidazopyrimidinylene group, an imidazopyridinylene group, a methylene group, an ethylene group, a propylene group, an iso-propylene group, a butylene group, a pentylene group, a hexylene group, a cyclobutylene group, a cyclopentylene group, and a cyclohexylene group; and

a phenylene group, a biphenylene group, a terphenylene group, a tetraphenylene group, an indenylene group, a naphthylene group, an acenaphthylene group, a fluorenylene group, a spiro-bifluorenylene group, a phenanthrenyl group, an anthracenylene group, a fluoranthrenylene group, a triphenylenylene group, a pyrenylene group, a chrysenylene group, a pyrrolylene group, an imidazolylene group, a pyrazolylene group, a triazolylene group, a pyridinylene group, a pyridazinylene group, a pyrimidinylene group, a pyridazinylene group, a triazinylene group, a furanylene group, a thienylene group, an oxazolylene group, an isoxazolylene group, a thiazolylene group, an isothiazolylene group, an oxadiazolylene group, an isoxadiazolylene group, a thiadiazolylene group, an iso-thiadiazolylene group, a pyranylene group, an imidazolylene group, a purinylene group, a quinolinylene group, an isoquinolinylene group, a benzoquinolinylene group, a phthalazinylene group, a naphthyridinylene group, a quinazolinylene group, a cinnolinylene group, a phenanthridinylene group, an acridinylene group, a phenanthrolinylene group, a phenazinylene group, a benzoxazinylene group, a benzothiazolylene group, benzimidazolylene group, an isoindolinylene group, an indolinylene group, a benzofuranylene group, a benzothiophenylene group, a carbazolylene group, an azacarbazolylene group, a dibenzofuranylene group, an azadibenzofuranylene group, a dibenzothiophenylene group, an azadibenzothiophenylene group, a benzocarbazolylene group, a naphthobenzocarbazolylene group, a naphthobenzofuranylene group, a naphthobenzothiophenylene group, an imidazopyrimidinylene group, an imidazopyridinylene group, a methylene group, an ethylene group, a propylene group, an iso-propylene group, a butylene group, a pentylene group, a hexylene group, a cyclobutylene group, a cyclopentylene group, and a cyclohexylene group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

For example, L₂ and L₃ may each independently be selected from a phenylene group, a fluorenylene group, a 9,9-diarylfluorenylene group, a 9,9-dialkylfluorenylene group, a spirobifluorenylene group, and a triphenylenylene group. In some embodiments, when L₂ or L₃ is a phenylene group, L₂ or L₃ may be an m-phenylene group or a p-phenylene group.

In some embodiments, L₂ or L₃ may be a substituted or unsubstituted heterocyclic group containing 5 to 60 ring-forming atoms. When L₂ or L₃ is a substituted or unsubstituted heterocyclic group containing 5 to 60 ring-forming atoms, L₂ or L₃ may be a substituted or unsubstituted condensed cyclic group including O or S as a heteroatom and three or more condensed rings, for example, a substituted or unsubstituted dibenzofuranylene group or a substituted or unsubstituted dibenzothiophenylene group.

In some embodiments, L₁ to L₃ may each independently be selected from the following structures:

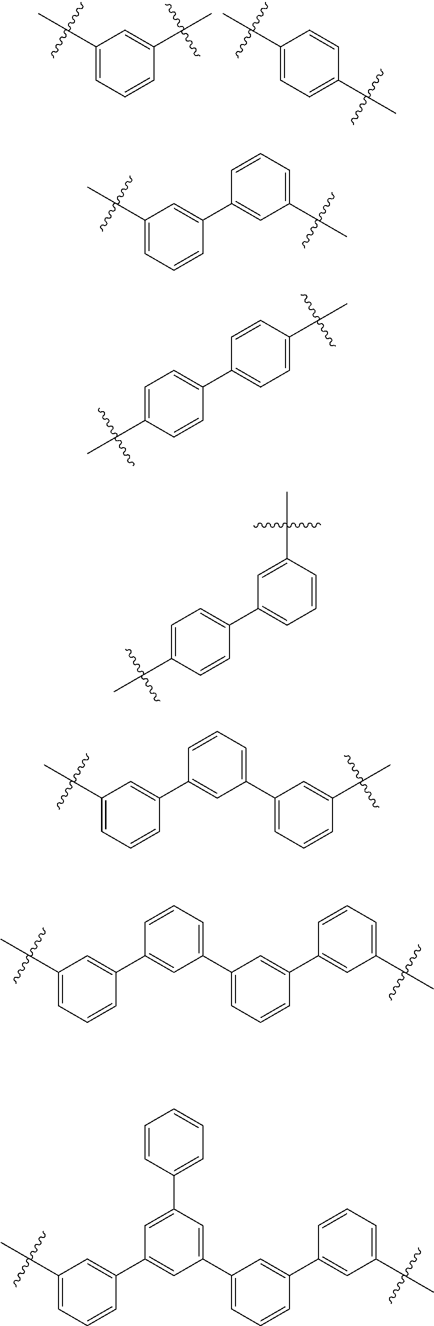

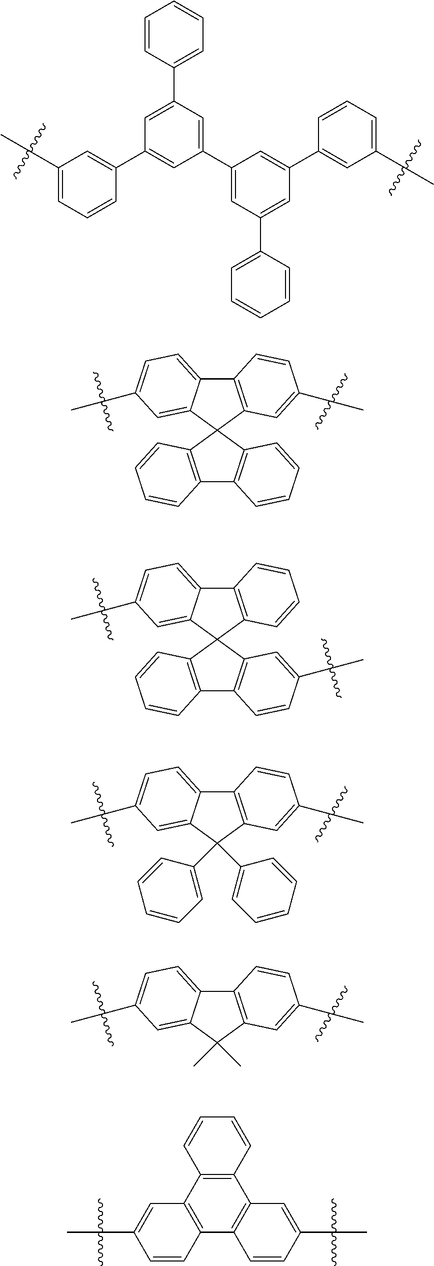

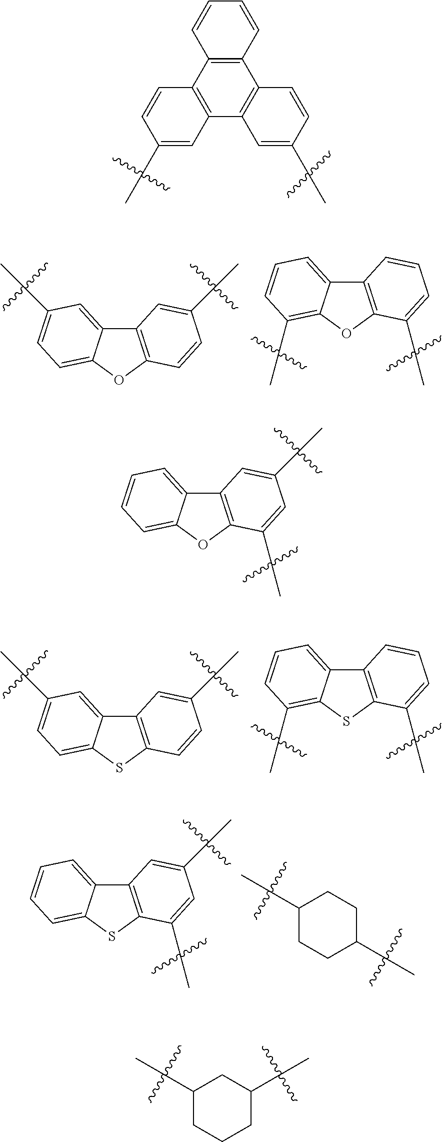

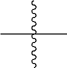

indicates a binding site to an adjacent atom.

In some embodiments, at least one selected from L₁ to L₃ may be selected from a substituted or unsubstituted C₅-C₆₀ carbocyclic group, a heterocyclic group containing 5 to 60 ring-forming atoms and O or S as a heteroatom, and a substituted or unsubstituted C₃-C₆₀ cycloalkylene group.

In Formula 1, b1 may be an integer from 1 to 5.

In some embodiments, b1 may be an integer from 1 to 3.

In Formula 1, b2 and b3 may each independently be an integer from 0 to 5.

In some embodiments, in Formula 1, b2 and b3 may each independently be an integer from 0 to 2.

In Formula 1, A₁ and A₂ may each independently be selected from a substituted or unsubstituted C₆-C₆₀ aryl group, a substituted or unsubstituted C₆-C₆₀ aryloxy group, a substituted or unsubstituted C₆-C₆₀ aryithio group, a substituted or unsubstituted C₁-C₆₀ heteroaryl group, a substituted or unsubstituted C₁-C₆₀ heteroaryloxy group, a substituted or unsubstituted C₁-C₆₀ heteroaryithio group, a substituted or unsubstituted monovalent non-aromatic condensed polycyclic group, a substituted or unsubstituted monovalent non-aromatic condensed heteropolycyclic group, and a substituted or unsubstituted C₅-C₆₀ heteroaryl group, provided that at least one selected from A₁ and A₂ may be a C₅-C₆₀ heteroaryl group.

In some embodiments, A₁ and A₂ may each independently be selected from a phenyl group, a biphenyl group, a terphenyl group, a tetraphenyl group, an indenyl group, a naphthyl group, an acenaphthyl group, a fluorenyl group, a spirobifluorenyl group, a phenanthrenyl group, a fluoranthenyl group, a triphenylene group, a pyrenyl group, a chrysenyl group, a pyrrolyl group, an imidazolyl group, a pyrazolyl group, a triazolyl group, a pyridinyl group, a pyrazinyl group, a pyrimidinyl group, a pyradizinyl group, a triazinyl group, a furanyl group, a thienyl group, a oxazolyl group, an isoxazolyl group, a thiazolyl group, an isothiazolyl group, an oxadiazolyl group, an isoxadiazolyl group, a thiadiazolyl group, an iso-thiadiazolyl group, a pyranyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzothiazolyl group, a benzimidazolyl group, an isoindolyl group, an indolyl group, a benzofuranyl group, a benzothiophenyl group, a carbazolyl group, a dibenzofuranyl group, a dibenzothiophenyl group, an azadibenzofuranyl group, a diazabenzofuranyl group, an azadibenzothiophenyl group, a diazabenzothiophenyl group, a benzocarbazolyl group, a naphthobenzofuranyl group, a naphthobenzothiophenyl group, an imidazopyrimidinyl group, and an imidazopyridinyl group; and

a phenyl group, a biphenyl group, a terphenyl group, a tetraphenyl group, an indenyl group, a naphthyl group, an acenaphthyl group, a fluorenyl group, a spirobifluorenyl group, a phenanthranelyne group, a fluoranthenyl group, a triphenylene group, a pyrenyl group, a chrysenyl group, a pyrrolyl group, an imidazolyl group, a pyrazolyl group, a triazolyl group, a pyridinyl group, a pyrazinyl group, a pyrimidinyl group, a pyradizinyl group, a triazinyl group, a furanyl group, a thienyl group, an oxazolyl group, an isoxazolyl group, a thiazolyl group, an isothiazolyl group, an oxadiazolyl group, an isoxadiazolyl group, a thiadiazolyl group, an iso-thiadiazolyl group, a pyranyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzothiazolyl group, a benzimidazolyl group, an isoindolyl group, an indolyl group, a benzofuranyl group, a benzothiophenyl group, a carbazolyl group, a dibenzofuranyl group, a dibenzothiophenyl group, an azadibenzofuranyl group, a diazabenzofuranyl group, an azadibenzothiophenyl group, a diazabenzothiophenyl group, a benzocarbazolyl group, a naphthobenzofuranyl group, a naphthobenzothiophenyl group, an imidazopyrimidinyl group, and an imidazopyridinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, and

at least one selected from A₁ and A₂ may be selected from a pyrrolyl group, an imidazolyl group, a pyrazolyl group, a triazolyl group, a pyridinyl group, a pyrazinyl group, a pyrimidinyl group, a pyradizinyl group, a triazinyl group, a furanyl group, a thienyl group, an oxazolyl group, an isoxazolyl group, a thiazolyl group, an isothiazolyl group, an oxadiazolyl group, an isoxadiazolyl group, a thiadiazolyl group, an isothiadiazolyl group, a pyranyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzothiazolyl group, a benzimidazolyl group, an isoindolyl group, an indolyl group, a benzofuranyl group, a benzothiophenyl group, a carbazolyl group, a dibenzofuranyl group, a dibenzothiophenyl group, an azadibenzofuranyl group, a diazabenzofuranyl group, an azadibenzothiophenyl group, a diazabenzothiophenyl group, a benzocarbazolyl group, a naphthobenzofuranyl group, a naphthobenzothiophenyl group, an imidazopyrimidinyl group, and an imidazopyridinyl group; and

a pyrrolyl group, an imidazolyl group, a pyrazolyl group, a triazolyl group, a pyridinyl group, a pyrazinyl group, a pyrimidinyl group, a pyradizinyl group, a triazinyl group, a furanyl group, a thienyl group, an oxazolyl group, an isoxazolyl group, a thiazolyl group, an isothiazolyl group, an oxadiazolyl group, an isoxadiazolyl group, a thiadiazolyl group, an iso-thiadiazolyl group, a pyranyl group, an indazolyl group, a purinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzoxazolyl group, a benzothiazolyl group, a benzimidazolyl group, an isoindolyl group, an indolyl group, a benzofuranyl group, a benzothiophenyl group, a carbazolyl group, a dibenzofuranyl group, a dibenzothiophenyl group, an azadibenzofuranyl group, a diazabenzofuranyl group, an azadibenzothiophenyl group, a diazabenzothiophenyl group, a benzocarbazolyl group, a naphthobenzofuranyl group, a naphthobenzothiophenyl group, an imidazopyrimidinyl group, and an imidazopyridinyl group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryioxy group, a C₆-C₆₀ aryithio group, a C₁-C₆₀ heteroaryl group, a C₁-C₆₀ heteroaryloxy group, a C₁-C₆₀ heteroarylthio group, a monovalent non-aromatic condensed polycyclic group, and a monovalent non-aromatic condensed heteropolycyclic group.

In some embodiments, at least one selected from A₁ and A₂ may be selected from Formulae 5-1 to 5-25:

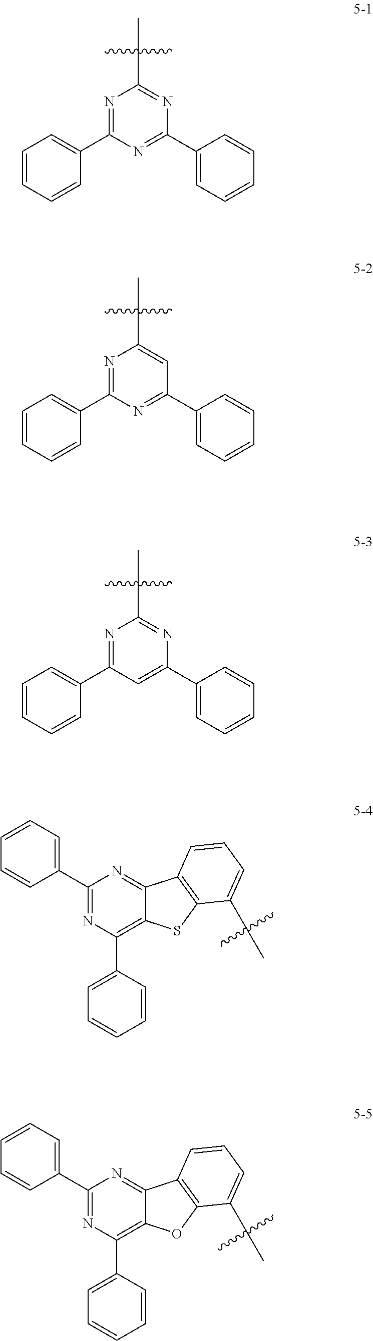 [SMILES: *c1nc(-c2ccccc2)nc(-c2ccccc2)n1 ; *c1cc(-c2ccccc2)nc(-c2ccccc2)n1 ; *c1nc(-c2ccccc2)cc(-c2ccccc2)n1 ; *c1cccc2c1sc1c(-c3ccccc3)nc(-c3ccccc3)nc12 ; *c1cccc2c1oc1c(-c3ccccc3)nc(-c3ccccc3)nc12]

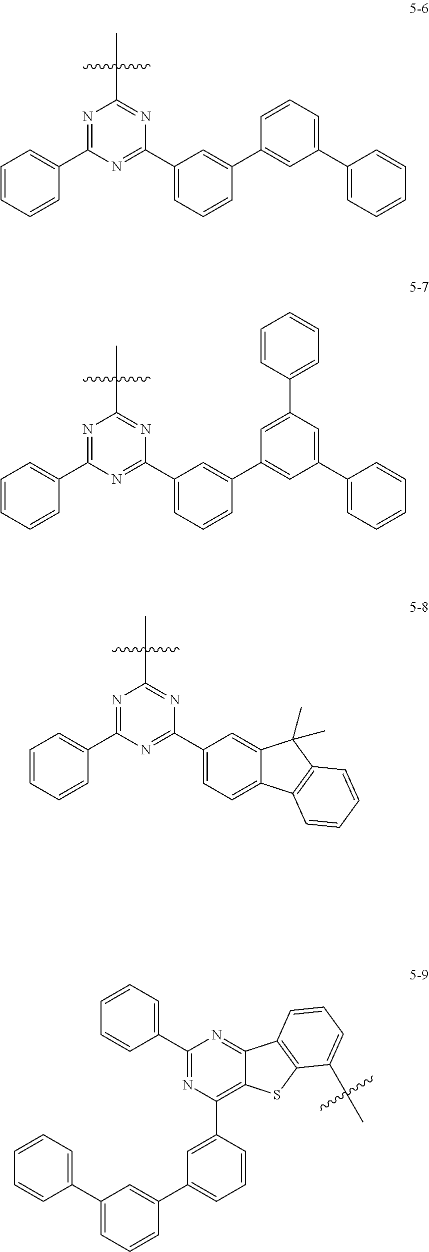 [SMILES: *c1nc(-c2ccccc2)nc(-c2cccc(-c3cccc(-c4ccccc4)c3)c2)n1 ; *c1nc(-c2ccccc2)nc(-c2cccc(-c3cc(-c4ccccc4)cc(-c4ccccc4)c3)c2)n1 ; *c1nc(-c2ccccc2)nc(-c2ccc3c(c2)C(C)(C)c2ccccc2-3)n1 ; *c1cccc2c1sc1c(-c3cccc(-c4cccc(-c5ccccc5)c4)c3)nc(-c3ccccc3)nc12]

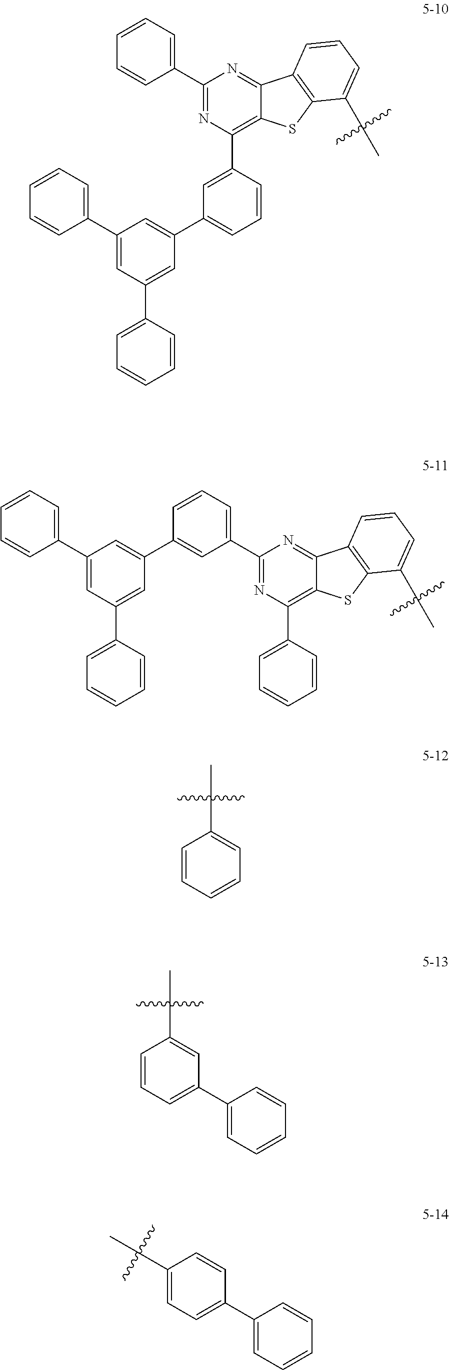 [SMILES: *c1cccc2c1sc1c(-c3cccc(-c4cc(-c5ccccc5)cc(-c5ccccc5)c4)c3)nc(-c3ccccc3)nc12 ; *c1cccc2c1sc1c(-c3ccccc3)nc(-c3cccc(-c4cc(-c5ccccc5)cc(-c5ccccc5)c4)c3)nc12 ; *c1ccccc1 ; *c1cccc(-c2ccccc2)c1 ; *c1ccc(-c2ccccc2)cc1]

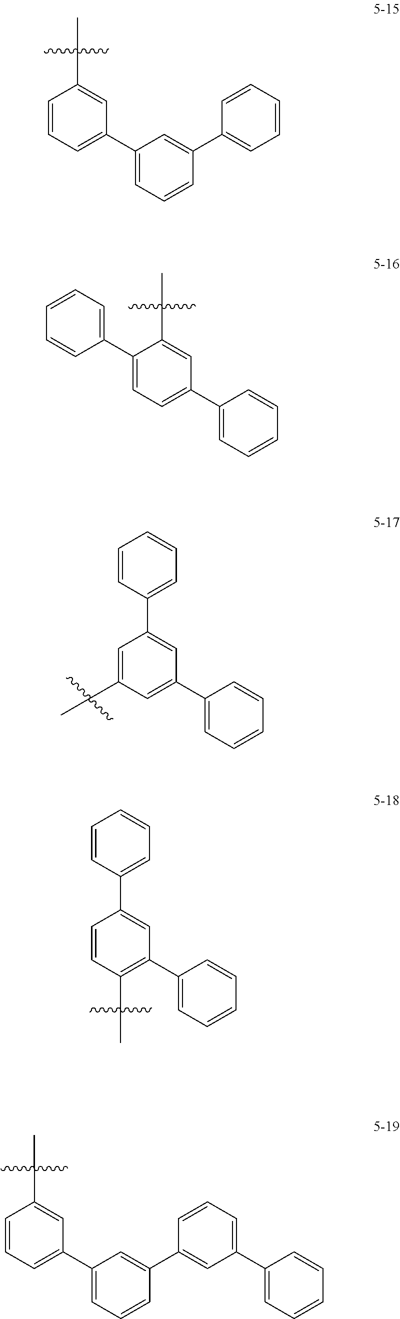 [SMILES: *c1cccc(-c2cccc(-c3ccccc3)c2)c1 ; *c1cc(-c2ccccc2)ccc1-c1ccccc1 ; *c1cc(-c2ccccc2)cc(-c2ccccc2)c1 ; *c1ccc(-c2ccccc2)cc1-c1ccccc1 ; *c1cccc(-c2cccc(-c3cccc(-c4ccccc4)c3)c2)c1]

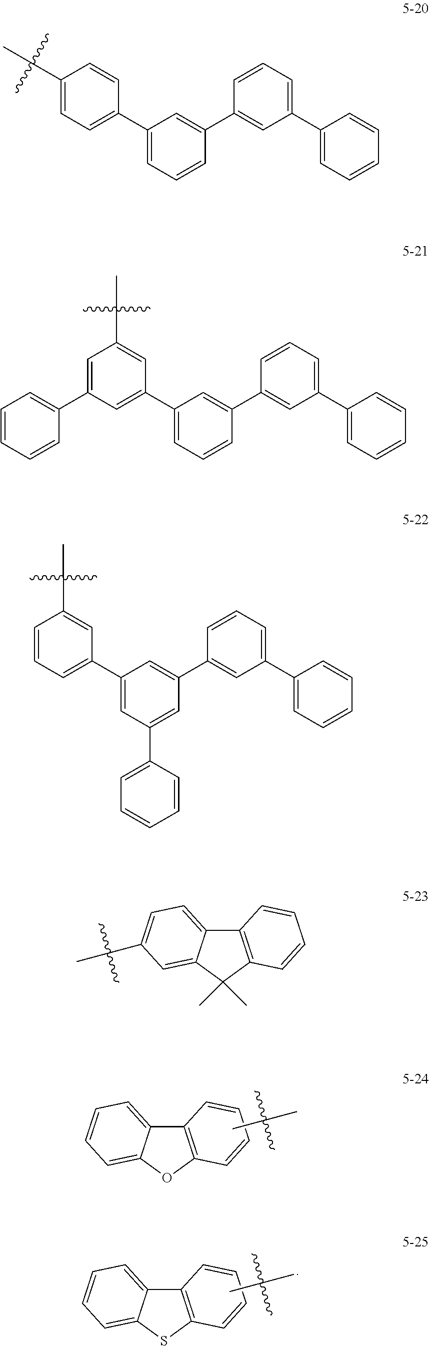

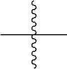

indicates a binding site to an adjacent atom.

In some embodiments, A₁ and A₂ may each independently be selected from Formulae 5-1 to 5-25.

In some embodiments, A₁ may be selected from Formulae 5-1 to 5-11.

In some embodiments, A₂ may be selected from Formulae 5-12 to 5-25.

In Formula 1, R₁ to R₆ may each independently be selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₆-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, and a monovalent non-aromatic condensed heteropolycyclic group.

In some embodiments, R₁ to R₆ may each independently be selected from hydrogen, —F, —Br, a substituted or unsubstituted C₁-C₂₀ alkyl group, a substituted or unsubstituted C₃-C₆₀ cycloalkyl group, a substituted or unsubstituted C₃-C₆₀ heterocycloalkyl group, a substituted or unsubstituted C₆-C₆₀ aryl group, a C₁-C₆₀ heteroaryl group, a monovalent non-aromatic condensed polycyclic group, and a monovalent non-aromatic condensed heteropolycyclic group.

For example, R₁ to R₆ may each independently be selected from hydrogen, —F, —Cl, —Br, —I, a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neopentyl group, a 1,2-dimethylpropyl group, an n-hexyl group, an iso-hexyl group, a 1,3-dimethylbutyl group, a 1-iso-propylpropyl group, a 1,2-dimethylbutyl group, a cyclopentyl group, a cyclohexyl group, a cycloheptyl group, a phenyl group, a naphthyl group, an anthracenyl group, a phenanthrenyl group, a pyrenyl group, a chrysenyl group, a triphenylenyl group, a fluorenyl group, a pyridinyl group, a pyrimidinyl group, a pyrazinyl group, a pyridazinyl group, a triazinyl group, a quinolinyl group, a quinazolinyl group, a quinoxalinyl group, an isoquinolinyl group, a carbazolyl group, a dibenzofuranyl group, a dibenzothiophenyl group, a biphenyl group, and a terphenyl group.

In some embodiments, R₁ to R₆ may each independently be selected from hydrogen, —F, —Cl, —Br, —I, a substituted or unsubstituted C₆-C₆₀ aryl group, a substituted or unsubstituted C₁-C₂₀ alkyl group, and a substituted or unsubstituted C₃-C₆₀ heterocycloalkyl group.

In Formula 1, a1, a4, and a5 may each independently be an integer from 0 to 3.

In some embodiments, a1, a4, and a5 may each independently be an integer from 1 to 3.

In Formula 1, a2, a3, and a6 may each independently be an integer from 0 to 4.

In some embodiments, a2 and a3 may each independently be an integer from 1 to 4. In some embodiments, a6 may be an integer from 1 to 4.

In some embodiments, the heterocyclic compound represented by Formula 1 may be represented by Formula 1A or Formula 1B:

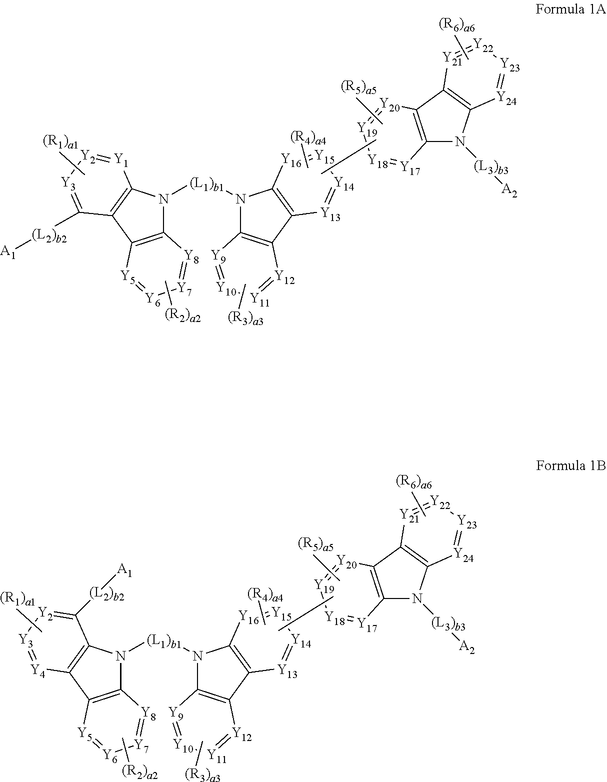

wherein, in Formulae 1A and 1B,

Y₁ to Y₂₄ may each independently be selected from N and C,

L₁ to L₃, A₁, A₂, b₁ to b₃, R₁ to R₆, and a1 to a6 may each be understood by referring to the description thereof provided herein.

In some embodiments, in Formulae 1A and 1B, at least one selected from Y₁ to Y₈ may be N.

In some embodiments, in Formulae 1A and 1B, Y₉ to Y₂₄ may each be C.

In some embodiments, in Formulae 1A and 1B, Y₁ to Y₂₄ may each be C.

In some embodiments, the heterocyclic compound represented by Formula 1 may be represented by Formula 10:

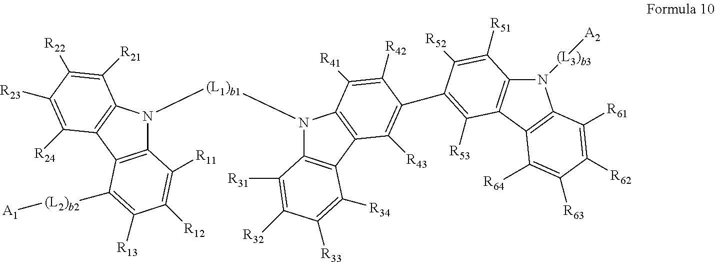

wherein, in Formula 10,

L₁ to L₃, A₁, A₂, and b₁ to b₃ may each be understood by referring to the description thereof provided herein,

R₁₁ to R₁₃ may each be understood by referring to the description of R₁ provided herein,

R₂₁ to R₂₄ may each be understood by referring to the description of R₂ provided herein,

R₃₁ to R₃₄ may each be understood by referring to the description of R₃ provided herein,

R₄₁ to R₄₃ may each be understood by referring to the description of R₄ provided herein,

R₅₁ to R₅₃ may each be understood by referring to the description of R₅ provided herein, and

R₆₁ to R₆₄ may each be understood by referring to the description of R₆ provided herein.

In some embodiments, R₁₁ to R₁₃, R₂₁, R₂₂, R₂₄, R₃₁, R₃₂, R₃₄, R₄₁ to R₄₃, R₅₁ to R₅₃, and R₆₁ to R₆₄ may each be hydrogen.

R₂₃ may be selected from hydrogen, a cyclopentyl group, a cyclohexyl group, a cycloheptyl group, a phenyl group, a naphthyl group, an anthracenyl group, a phenanthrenyl group, a pyrenyl group, a chrysenyl group, a triphenylenyl group, a fluorenyl group, a pyridinyl group, a pyrimidinyl group, a pyrazinyl group, a pyridazinyl group, a triazinyl group, a quinolinyl group, a quinazolinyl group, a quinoxalinyl group, an isoquinolinyl group, a carbazolyl group, a dibenzofuranyl group, a dibenzothiophenyl group, a biphenyl group, and a terphenyl group, and

R₃₃ may be selected from a cyclopentyl group, a cyclohexyl group, a cycloheptyl group, a phenyl group, a naphthyl group, an anthracenyl group, a phenanthrenyl group, a pyrenyl group, a chrysenyl group, a triphenylenyl group, a fluorenyl group, a pyridinyl group, a pyrimidinyl group, a pyrazinyl group, a pyridazinyl group, a triazinyl group, a quinolinyl group, a quinazolinyl group, a quinoxalinyl group, an isoquinolinyl group, a carbazolyl group, a dibenzofuranyl group, a dibenzothiophenyl group, a biphenyl group, and a terphenyl group.

In some embodiments, the heterocyclic compound represented by Formula 1 may be selected from Compounds 1 to 36, but embodiments are not limited thereto:

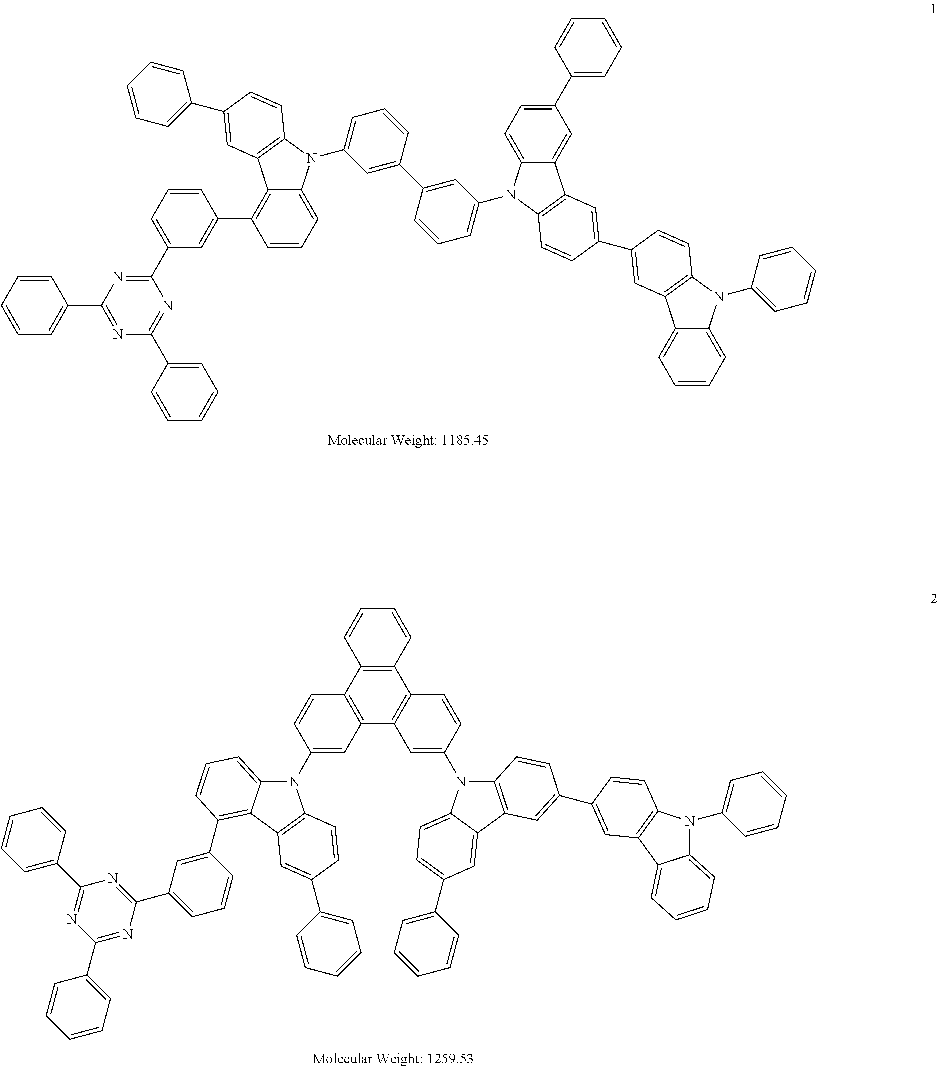 [SMILES: c1ccc(-c2ccc3c(c2)c2cc(-c4ccc5c(c4)c4ccccc4n5-c4ccccc4)ccc2n3-c2cccc(-c3cccc(-n4c5ccc(-c6ccccc6)cc5c5c(-c6cccc(-c7nc(-c8ccccc8)nc(-c8ccccc8)n7)c6)cccc54)c3)c2)cc1 ; c1ccc(-c2ccc3c(c2)c2cc(-c4ccc5c(c4)c4ccccc4n5-c4ccccc4)ccc2n3-c2ccc3c4ccccc4c4ccc(-n5c6ccc(-c7ccccc7)cc6c6c(-c7cccc(-c8nc(-c9ccccc9)nc(-c9ccccc9)n8)c7)cccc65)cc4c3c2)cc1]

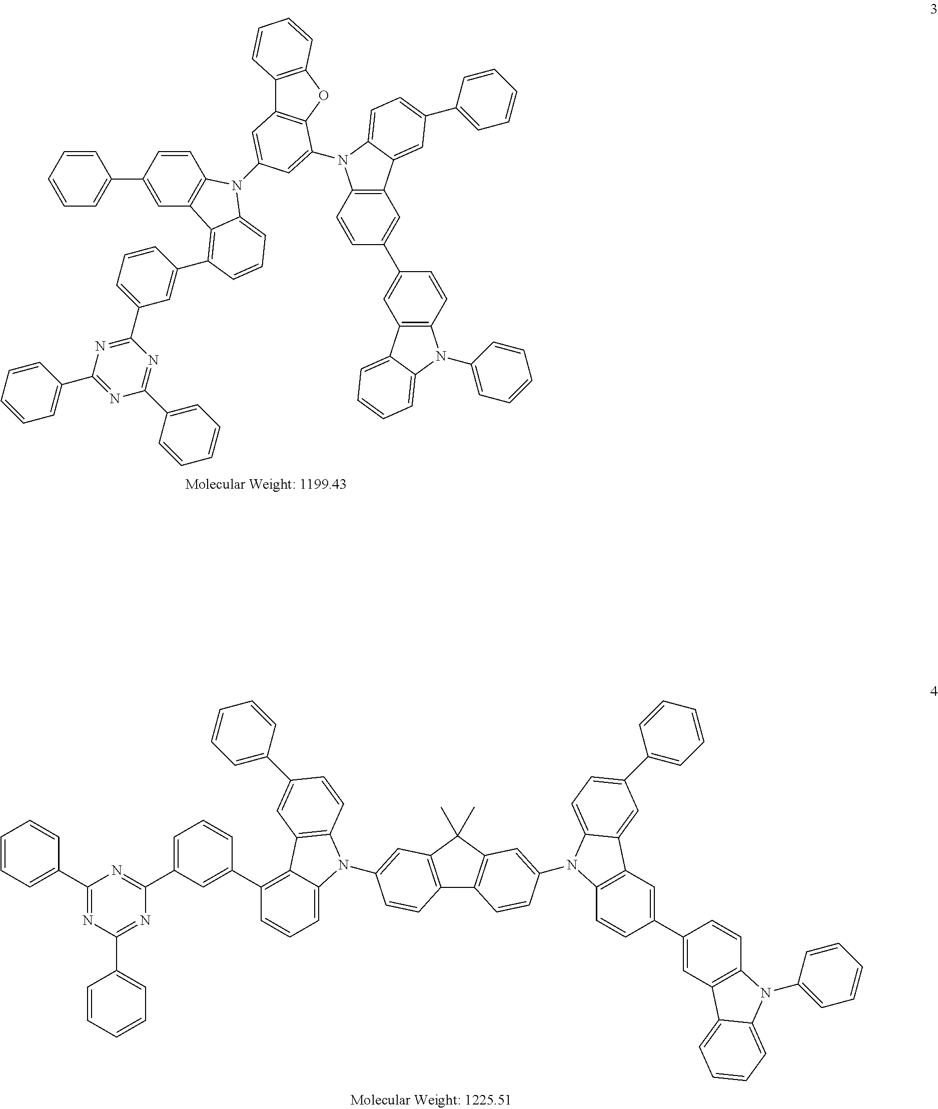 [SMILES: c1ccc(-c2ccc3c(c2)c2cc(-c4ccc5c(c4)c4ccccc4n5-c4ccccc4)ccc2n3-c2cc(-n3c4ccc(-c5ccccc5)cc4c4c(-c5cccc(-c6nc(-c7ccccc7)nc(-c7ccccc7)n6)c5)cccc43)cc3c2oc2ccccc23)cc1 ; CC1(C)c2cc(-n3c4ccc(-c5ccccc5)cc4c4cc(-c5ccc6c(c5)c5ccccc5n6-c5ccccc5)ccc43)ccc2-c2ccc(-n3c4ccc(-c5ccccc5)cc4c4c(-c5cccc(-c6nc(-c7ccccc7)nc(-c7ccccc7)n6)c5)cccc43)cc21]

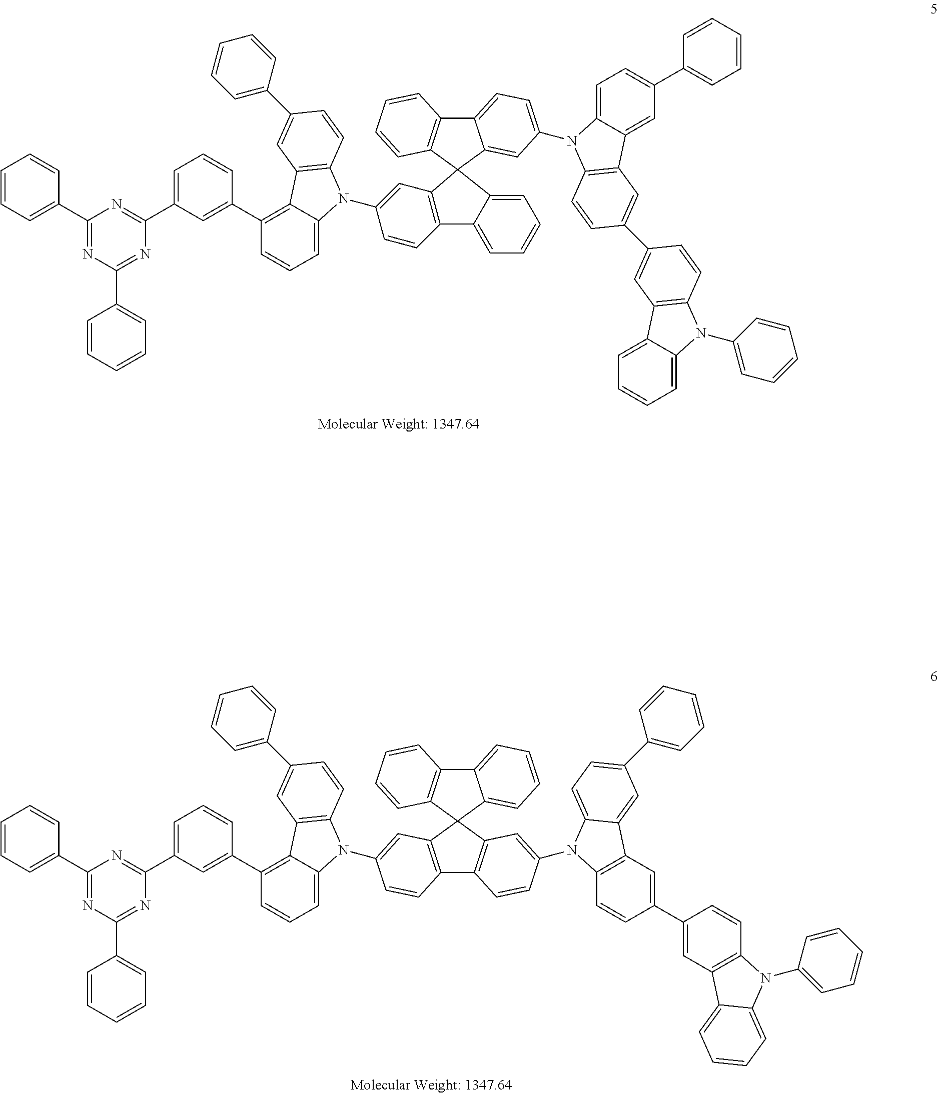 [SMILES: c1ccc(-c2ccc3c(c2)c2cc(-c4ccc5c(c4)c4ccccc4n5-c4ccccc4)ccc2n3-c2ccc3c(c2)C2(c4ccccc4-3)c3ccccc3-c3ccc(-n4c5ccc(-c6ccccc6)cc5c5c(-c6cccc(-c7nc(-c8ccccc8)nc(-c8ccccc8)n7)c6)cccc54)cc32)cc1 ; c1ccc(-c2ccc3c(c2)c2cc(-c4ccc5c(c4)c4ccccc4n5-c4ccccc4)ccc2n3-c2ccc3c(c2)C2(c4ccccc4-c4ccccc42)c2cc(-n4c5ccc(-c6ccccc6)cc5c5c(-c6cccc(-c7nc(-c8ccccc8)nc(-c8ccccc8)n7)c6)cccc54)ccc2-3)cc1]

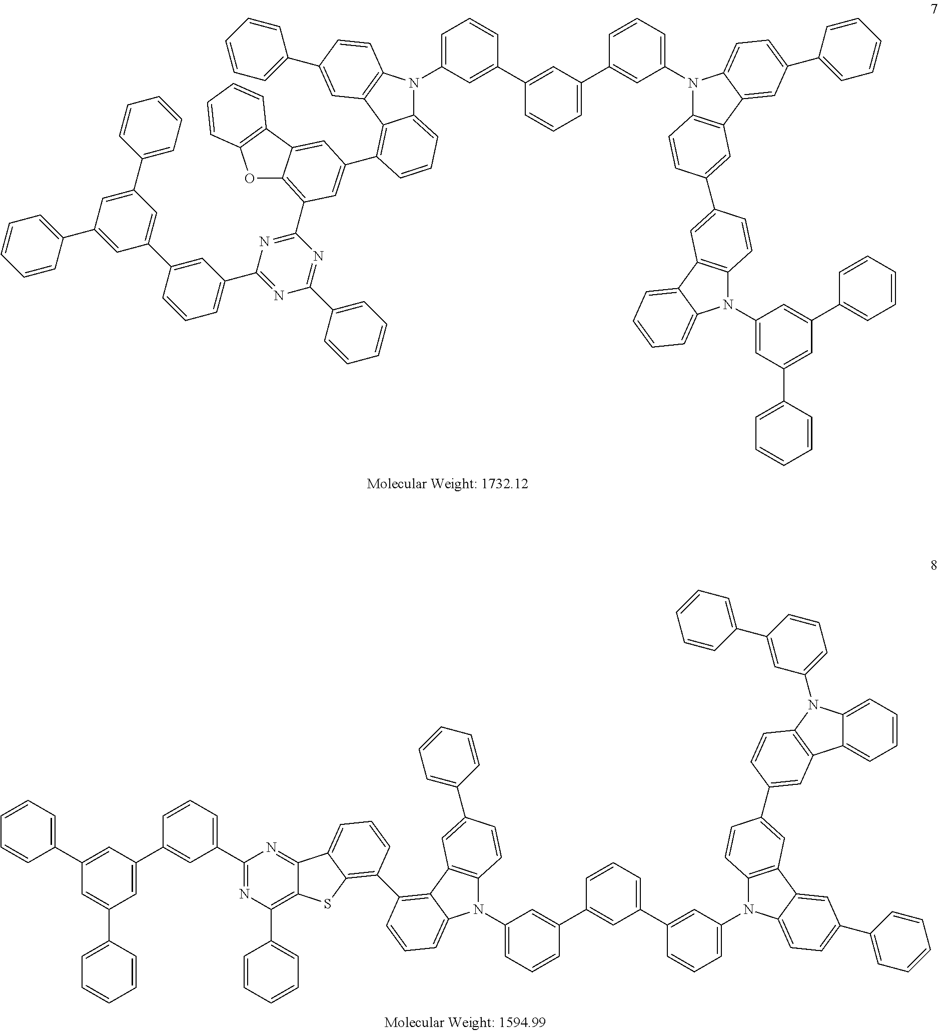

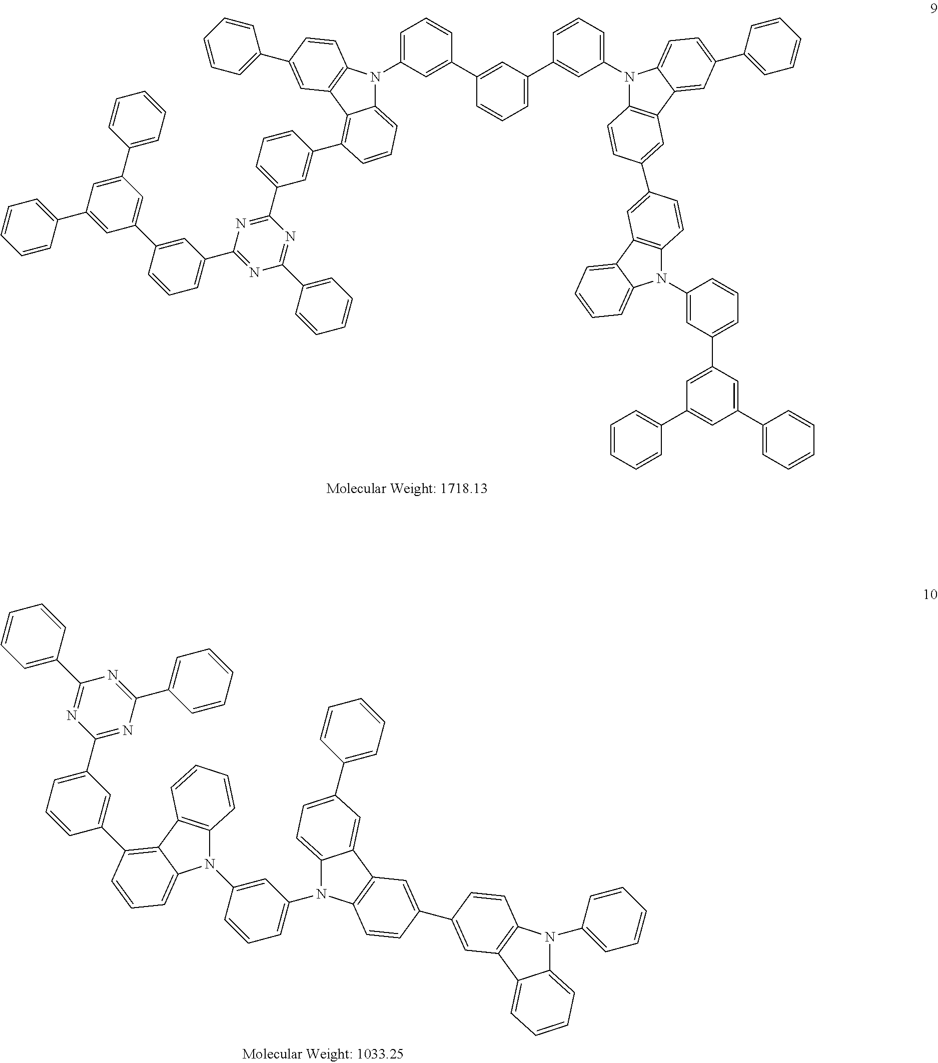 [SMILES: c1ccc(-c2cc(-c3ccccc3)cc(-c3cccc(-c4nc(-c5ccccc5)nc(-c5cccc(-c6cccc7c6c6cc(-c8ccccc8)ccc6n7-c6cccc(-c7cccc(-c8cccc(-n9c%10ccc(-c%11ccccc%11)cc%10c%10cc(-c%11ccc%12c(c%11)c%11ccccc%11n%12-c%11cccc(-c%12cc(-c%13ccccc%13)cc(-c%13ccccc%13)c%12)c%11)ccc%109)c8)c7)c6)c5)n4)c3)c2)cc1 ; c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6nc(-c7ccccc7)nc(-c7ccccc7)n6)c5)cccc43)c2)cc1]

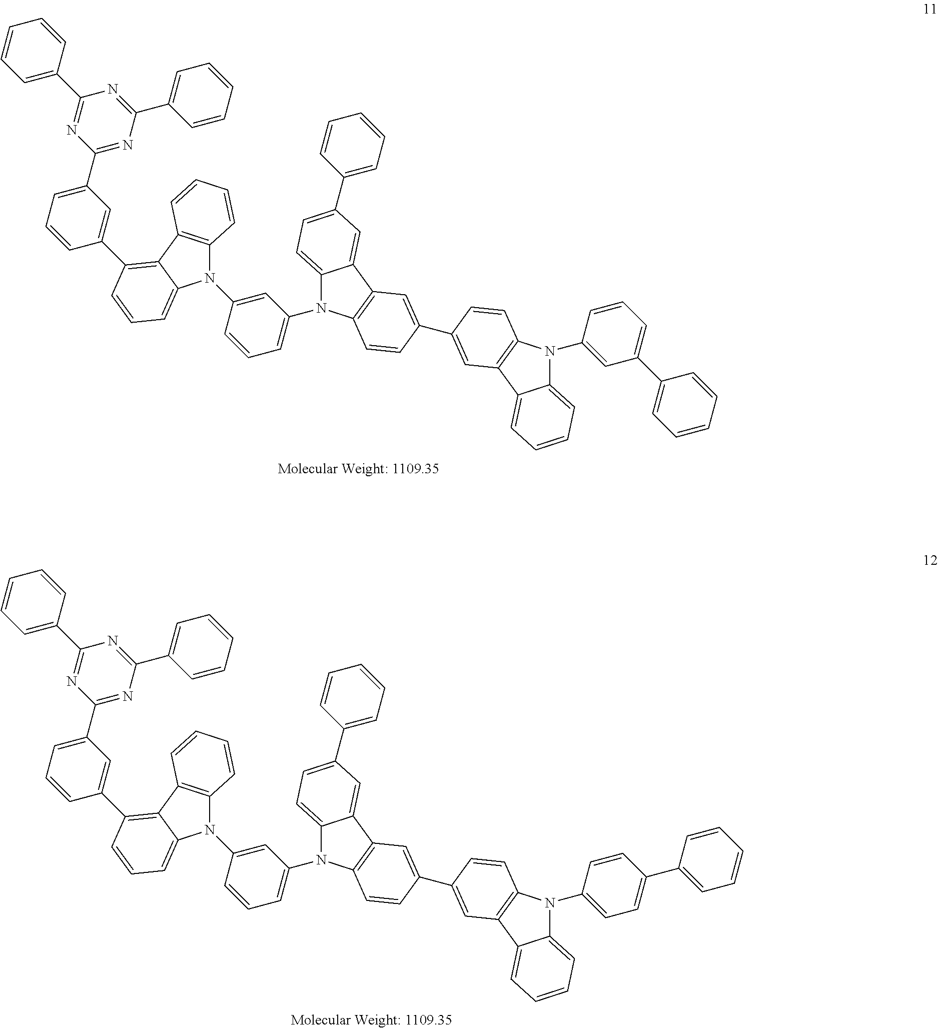 [SMILES: c1ccc(-c2cccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)c2)cc1 ; c1ccc(-c2ccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)cc2)cc1]

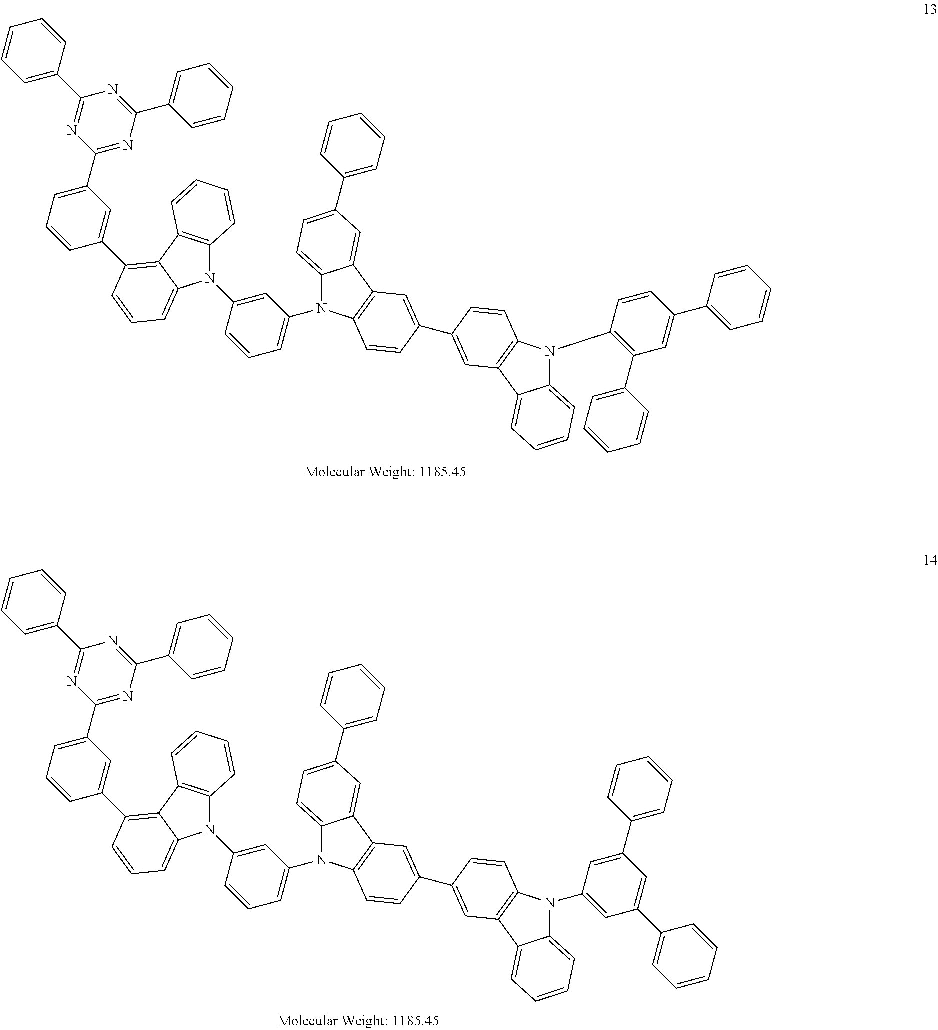 [SMILES: c1ccc(-c2ccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)c(-c3ccccc3)c2)cc1 ; c1ccc(-c2cc(-c3ccccc3)cc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)c2)cc1]

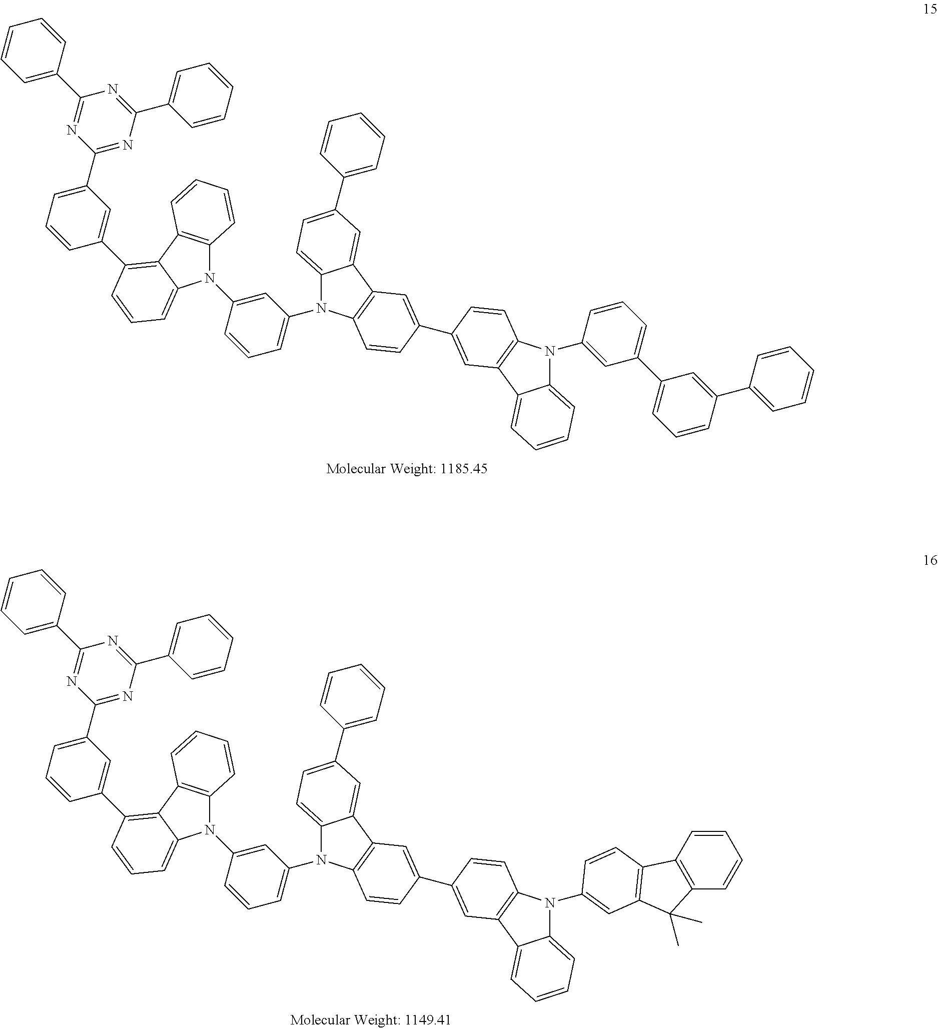 [SMILES: c1ccc(-c2cccc(-c3cccc(-n4c5ccccc5c5cc(-c6ccc7c(c6)c6cc(-c8ccccc8)ccc6n7-c6cccc(-n7c8ccccc8c8c(-c9cccc(-c%10nc(-c%11ccccc%11)nc(-c%11ccccc%11)n%10)c9)cccc87)c6)ccc54)c3)c2)cc1 ; CC1(C)c2ccccc2-c2ccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)cc21]

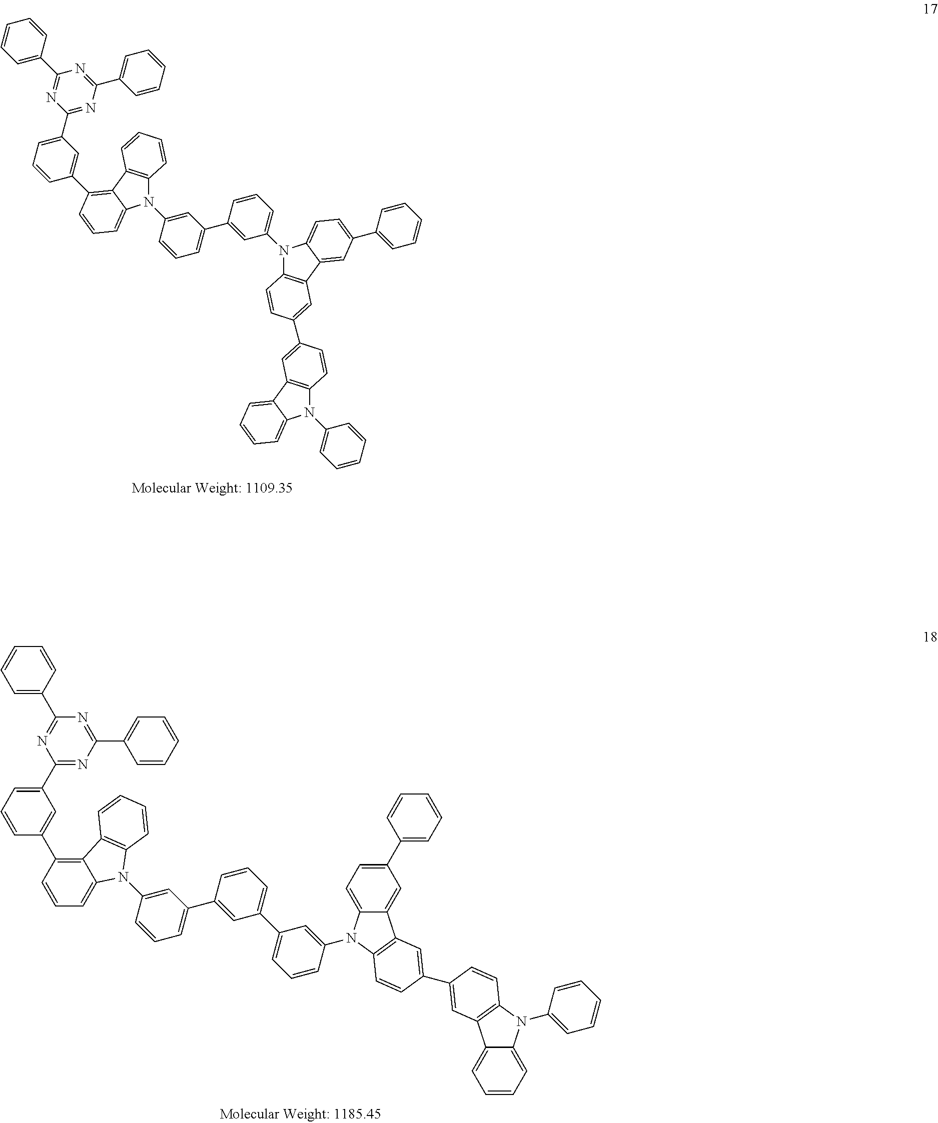 [SMILES: c1ccc(-c2ccc3c(c2)c2cc(-c4ccc5c(c4)c4ccccc4n5-c4ccccc4)ccc2n3-c2cccc(-c3cccc(-n4c5ccccc5c5c(-c6cccc(-c7nc(-c8ccccc8)nc(-c8ccccc8)n7)c6)cccc54)c3)c2)cc1 ; c1ccc(-c2ccc3c(c2)c2cc(-c4ccc5c(c4)c4ccccc4n5-c4ccccc4)ccc2n3-c2cccc(-c3cccc(-c4cccc(-n5c6ccccc6c6c(-c7cccc(-c8nc(-c9ccccc9)nc(-c9ccccc9)n8)c7)cccc65)c4)c3)c2)cc1]

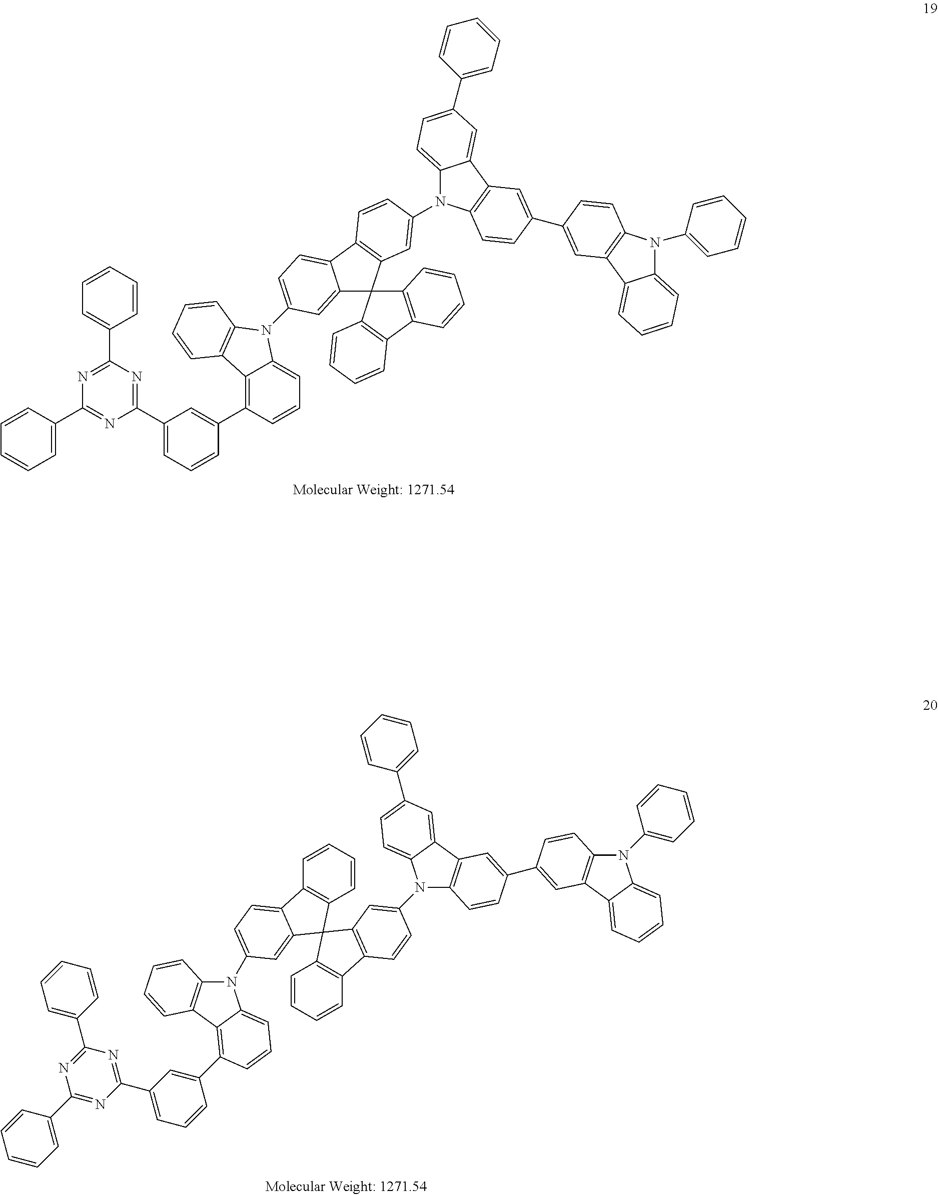 [SMILES: c1ccc(-c2ccc3c(c2)c2cc(-c4ccc5c(c4)c4ccccc4n5-c4ccccc4)ccc2n3-c2ccc3c(c2)C2(c4ccccc4-c4ccccc42)c2cc(-n4c5ccccc5c5c(-c6cccc(-c7nc(-c8ccccc8)nc(-c8ccccc8)n7)c6)cccc54)ccc2-3)cc1 ; c1ccc(-c2ccc3c(c2)c2cc(-c4ccc5c(c4)c4ccccc4n5-c4ccccc4)ccc2n3-c2ccc3c(c2)C2(c4ccccc4-3)c3ccccc3-c3ccc(-n4c5ccccc5c5c(-c6cccc(-c7nc(-c8ccccc8)nc(-c8ccccc8)n7)c6)cccc54)cc32)cc1]

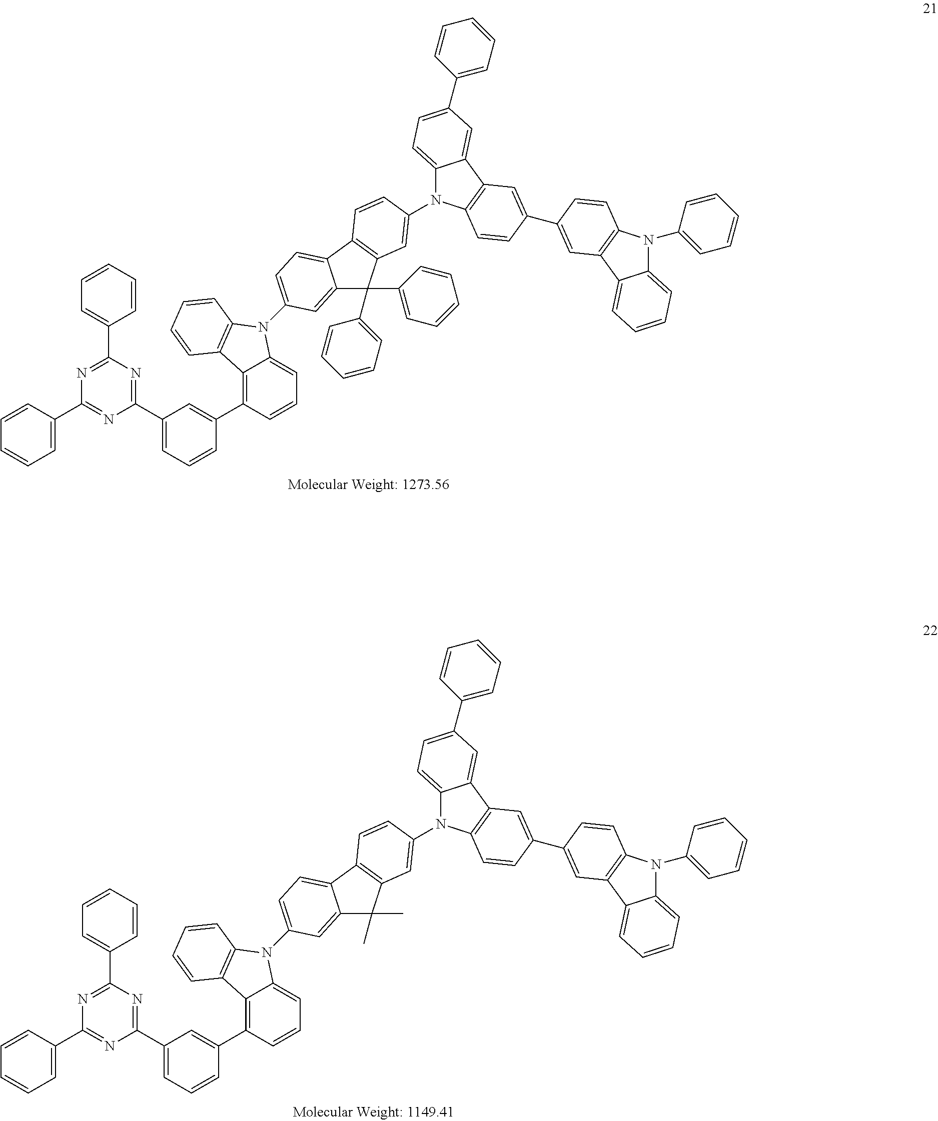 [SMILES: c1ccc(-c2ccc3c(c2)c2cc(-c4ccc5c(c4)c4ccccc4n5-c4ccccc4)ccc2n3-c2ccc3c(c2)C(c2ccccc2)(c2ccccc2)c2cc(-n4c5ccccc5c5c(-c6cccc(-c7nc(-c8ccccc8)nc(-c8ccccc8)n7)c6)cccc54)ccc2-3)cc1 ; CC1(C)c2cc(-n3c4ccc(-c5ccccc5)cc4c4cc(-c5ccc6c(c5)c5ccccc5n6-c5ccccc5)ccc43)ccc2-c2ccc(-n3c4ccccc4c4c(-c5cccc(-c6nc(-c7ccccc7)nc(-c7ccccc7)n6)c5)cccc43)cc21]

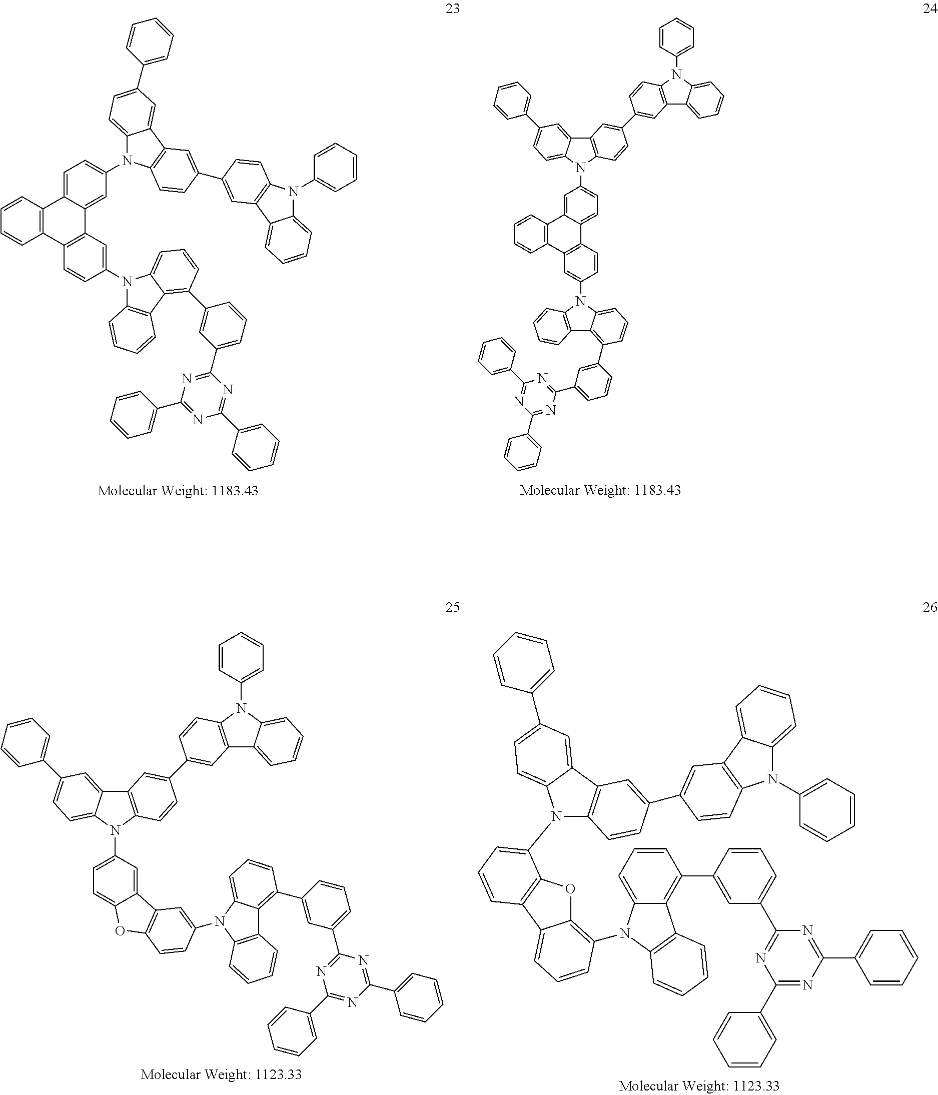

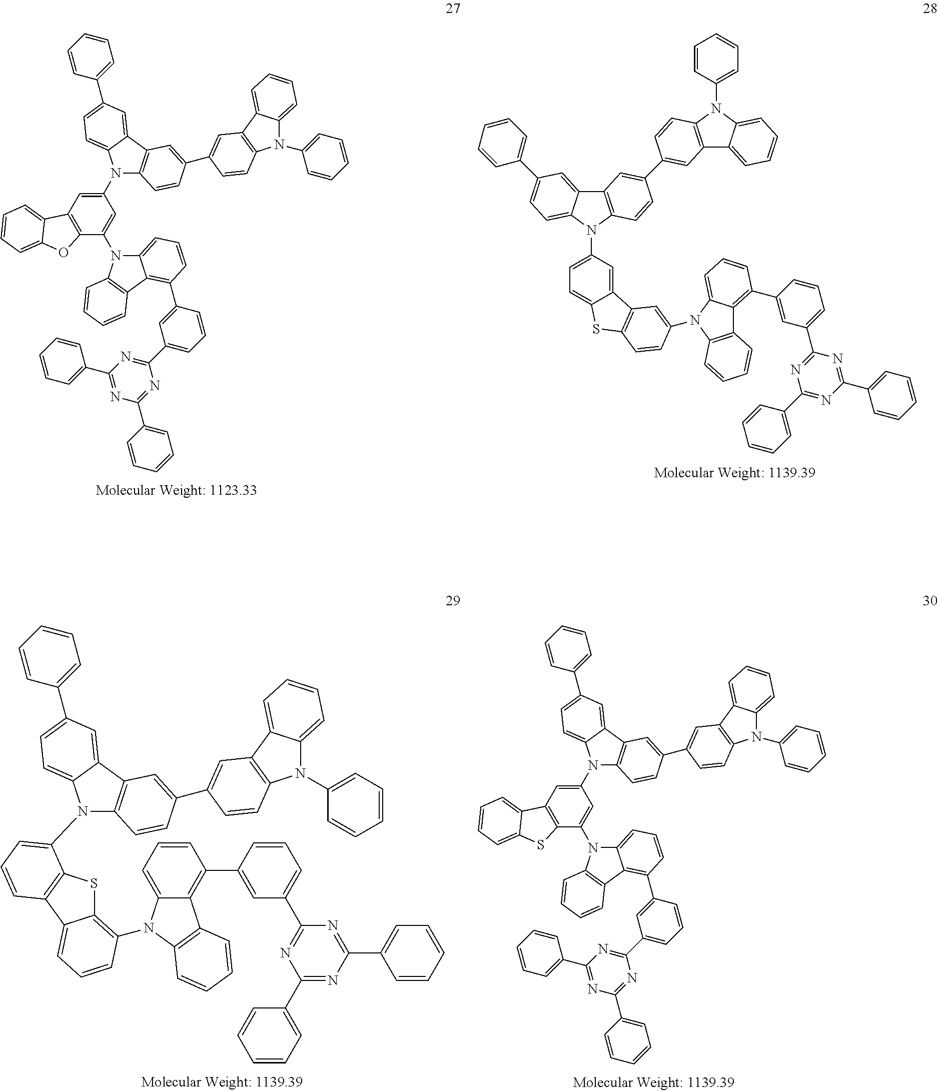

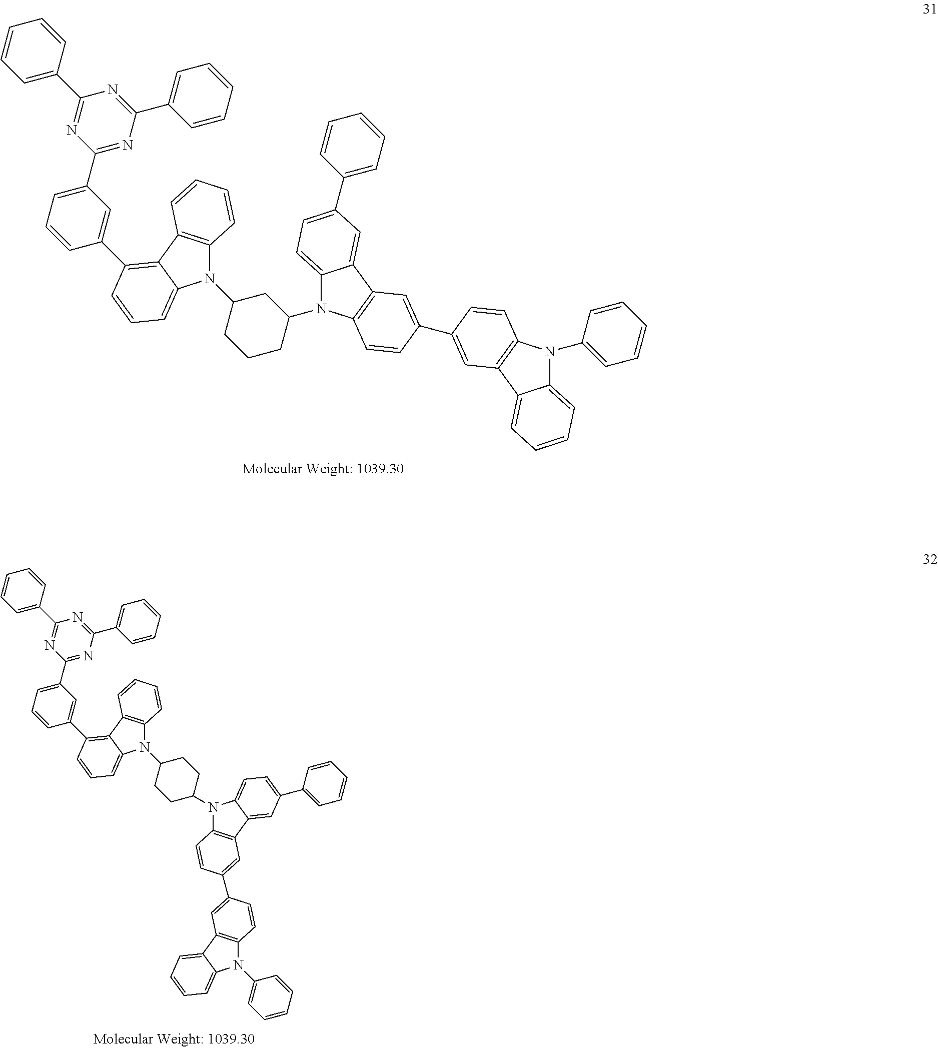 [SMILES: c1ccc(-c2ccc3c(c2)c2cc(-c4ccc5c(c4)c4ccccc4n5-c4ccccc4)ccc2n3C2CCCC(n3c4ccccc4c4c(-c5cccc(-c6nc(-c7ccccc7)nc(-c7ccccc7)n6)c5)cccc43)C2)cc1 ; c1ccc(-c2ccc3c(c2)c2cc(-c4ccc5c(c4)c4ccccc4n5-c4ccccc4)ccc2n3C2CCC(n3c4ccccc4c4c(-c5cccc(-c6nc(-c7ccccc7)nc(-c7ccccc7)n6)c5)cccc43)CC2)cc1]

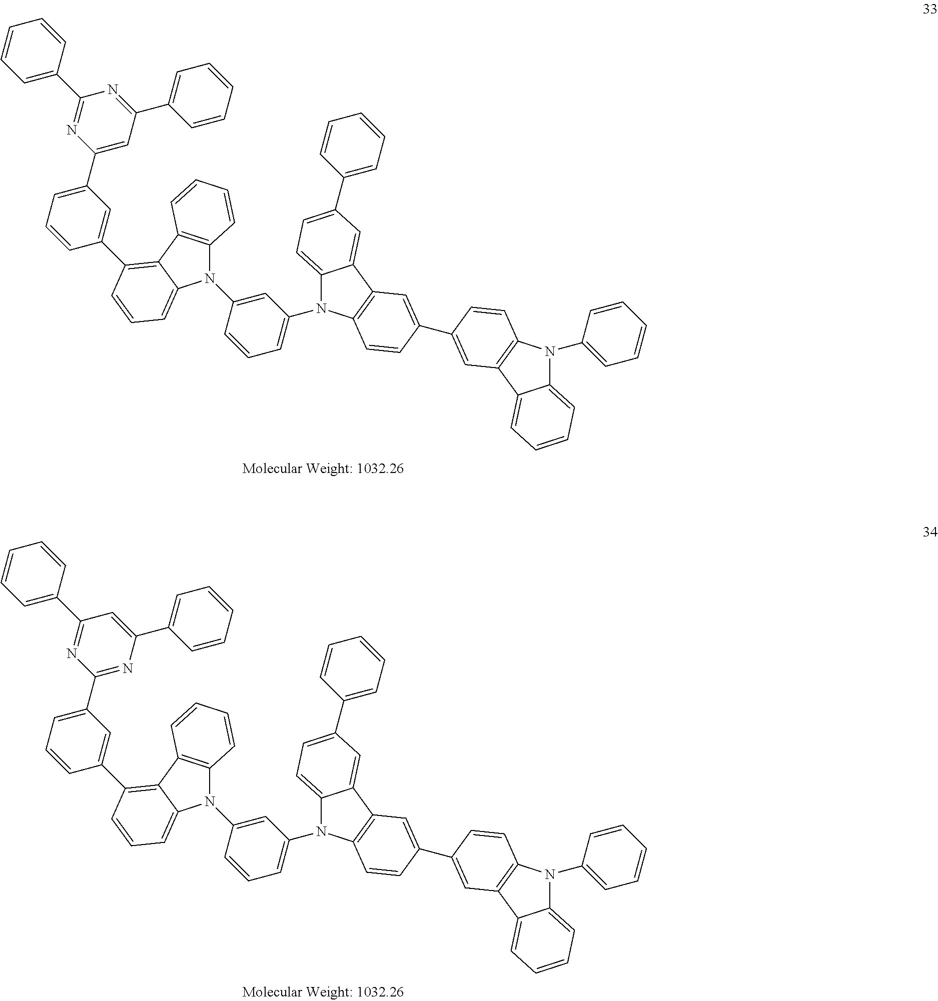 [SMILES: c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6cc(-c7ccccc7)nc(-c7ccccc7)n6)c5)cccc43)c2)cc1 ; c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6nc(-c7ccccc7)cc(-c7ccccc7)n6)c5)cccc43)c2)cc1]

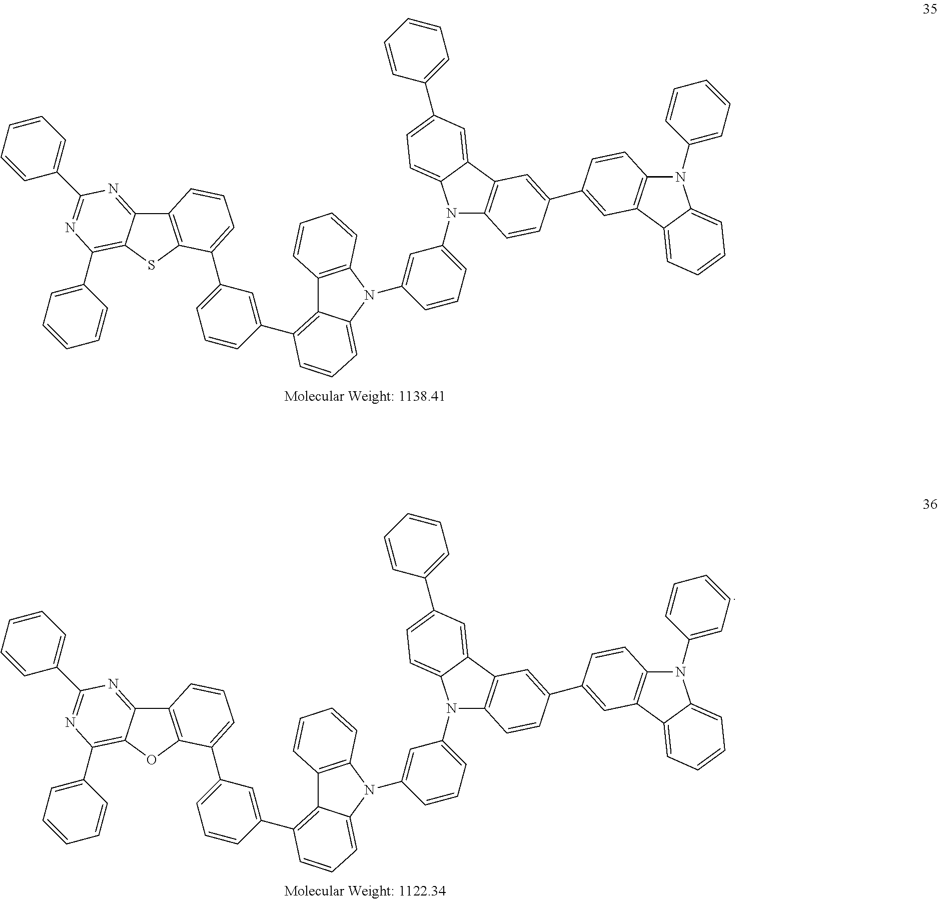 [SMILES: c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6cccc7c6sc6c(-c8ccccc8)nc(-c8ccccc8)nc67)c5)cccc43)c2)cc1 ; c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6cccc7c6oc6c(-c8ccccc8)nc(-c8ccccc8)nc67)c5)cccc43)c2)cc1]

An organic light-emitting device may include an organic layer including an emission layer, wherein the organic layer may include a stacked structure of, for example, a hole injection layer, a hole transport layer, an emission layer, an electron transport layer, and an electron injection layer. Deposition or coating methods are widely known as a method of forming such an organic layer. Among these methods, when a coating method (solution coating, also known as wet layer coating) is used, manufacture of a device of a large area may be possible with a low cost. Thus, a high productivity may be expected. However, when a coating method is used, a material for forming an organic layer is required to have solubility in a solvent and durability in drying a coating liquid.

Also, a device in which an emission layer is formed by using a coating method may tend to have a low current efficiency and short emission lifespan in general, as compared with a device in which an emission layer is formed by using deposition. This may be because materials of layers formed by coating may easily transfer to adjacent layers via interfaces, thus deteriorating performances of each layer.

The heterocyclic compound according to one or more embodiments, as shown in Formula 1, may be a compound, in which two carbazoles may be directly connected to each other, including at least one carbazole or azacarbazole having an N-substituted core, to which at least one heterocyclic group may be further substituted. Accordingly, the heterocyclic compound may be used in manufacture of an organic light-emitting device and may provide a high performance device which may exhibit high current efficiency and long lifespan.

While not wishing to be bound by theory, it is understood that as the heterocyclic compound has a relatively high molecular weight due to the structure represented by Formula 1, transfer of materials at an adjacent interfaces may be prevented.

For example, when the heterocyclic compound is used in forming an emission layer of an organic layer of an organic light-emitting device, transfer of emission layer materials at an interface between the emission layer and a hole transport layer may be prevented. For example, when the heterocyclic compound is used in forming an organic layer other than an emission layer, e.g., a hole transport layer, transfer of materials at an interface between the hole transport layer and the emission layer and/or a hole injection layer may be prevented. As such, when the heterocyclic compound is used in forming each layer, transfer of materials at adjacent layers may be prevented, thereby stably maintaining a function of each layer. Thus, an organic light-emitting device having a high efficiency and long lifespan may be provided.

In some embodiments, the heterocyclic compound represented by Formula 1 may have a molecular weight in a range of about 1,000 Daltons (Da) to about 3,000 Da. In some embodiments, the heterocyclic compound represented by Formula 1 may have a molecular weight of about 1,000 Da or greater and less than about 3,000 Da. In some embodiments, the heterocyclic compound represented by Formula 1 may have a molecular weight of about 1,000 Da or greater and less than about 2,500 Da. In some embodiments, the heterocyclic compound represented by Formula 1 may have a molecular weight of about 1,000 Da or greater and less than about 2,000 Da. In some embodiments, the heterocyclic compound represented by Formula 1 may have a molecular weight of about 1,200 Da or greater and less than about 1,800 Da.

As the heterocyclic compound has a molecular weight range as described above, the heterocyclic compound has a relatively high molecular weight, and a size of the heterocyclic compound is relatively large such that transfer of molecules between adjacent interfaces may be prevented. Accordingly, functions of a layer including the heterocyclic compound and another layer adjacent thereto may be stably maintained. In particular, when the heterocyclic compound is used as a host material in an emission layer, transfer of materials at an interface between the emission layer and a hole transport layer may be prevented. Thus, an organic light-emitting device having an excellent current efficiency and emission lifespan may be provided.

In some embodiments, a composition for an organic light-emitting device may include at least one heterocyclic compound represented by Formula 1.

In some embodiments, the composition for an organic light-emitting device may further include a solvent, and a content of the heterocyclic compound in the composition for an organic light-emitting device may be 0.01 percent by mass (mass %) or greater.

Organic Light-Emitting Device

Hereinafter, with reference to the FIGURE, an embodiment of an organic light-emitting device will be described in detail. The FIGURE is a schematic view of an organic light-emitting device according to an embodiment.

An organic light-emitting device 100 according to an example embodiment may include a substrate 110, a first electrode 120 on the substrate 110, a hole injection layer 130 on the first electrode 120, a hole transport layer 140 on the hole injection layer 130, an emission layer 150 on the hole transport layer 140, an electron transport layer 160 on the emission layer 150, an electron injection layer 170 on the electron transport layer 160, and a second electrode 180 on the electron injection layer 170.

In the organic light-emitting device 100, the heterocyclic compound represented by Formula 1 may be, for example, included in at least one organic layer (e.g., at least one organic layer selected from the hole injection layer 130, the hole transport layer 140, the emission layer 150, the electron transport layer 160, and the electron injection layer 170 between the first electrode 120 and the second electrode 180. In some embodiments, the heterocyclic compound represented by Formula 1 may be included in the emission layer 150 as a hole transport host. In some embodiments, the heterocyclic compound represented by Formula 1 may be included in an organic layer other than the emission layer 150. For example, the heterocyclic compound represented by Formula 1 may be included in the hole injection layer 130 and/or the hole transport layer as a hole transport material.

The term “organic layer” as used herein refers to a single and/or a plurality of layers between the first electrode and the second electrode in an organic light-emitting device. The “organic layer” may include not only organic compounds but also organometallic compounds including metals.

As used herein, the expression “(for example, the organic layer) including at least one heterocyclic compound” means that “(the organic layer) including a heterocyclic compound represented by Formula 1, or at least two different heterocyclic compounds represented by Formula 1”.

For example, the organic layer may include Compound 1 only as the heterocyclic compound. In this embodiment, Compound 1 may be included in the emission layer of the organic light-emitting device. In some embodiments, Compounds 1 and 2 may be included in the organic layer as the heterocyclic compounds. In this embodiment, Compounds 1 and 2 may both be included in the same layer (for example, both Compounds 1 and 2 may be included in the emission layer).

The substrate 110 may be any suitable substrate generally used in organic light-emitting devices. For example, the substrate 110 may be a glass substrate, a silicon substrate, or a transparent plastic substrate having excellent mechanical strength, thermal stability, transparency, surface smoothness, ease of handling, and water repellency, but embodiments are not limited thereto.

The first electrode 120 may be formed on the substrate 110. The first electrode 120 may be an anode, and be formed of a material with a relatively high work function selected from a metal, an alloy, or a conductive compound, for facilitating hole injection. The first electrode 120 may be a reflective electrode, a semi-transmissive electrode, or a transmissive electrode. The first electrode 120 may have a single-layered structure or a multi-layered structure including a plurality of layers. For example, the first electrode 120 may be a transparent electrode formed of indium tin oxide (ITO), indium zinc oxide (IZO), tin oxide (SnO₂), zinc oxide (ZnO) having excellent transparency and conductivity. The first electrode 120 may be a reflective electrode that may be formed by stacking magnesium (Mg), aluminum (Al) aluminum-lithium (Al—Li), calcium (Ca), magnesium-indium (Mg—In), magnesium-silver (Mg—Ag) on the transparent electrode. For example, the first electrode 120 may have a triple-layer structure of ITO/Ag/ITO, but embodiments are not limited thereto.

A hole transport region may be formed on the first electrode 120.

The hole transport region may include at least one selected from the hole injection layer 130, the hole transport layer 140, an electron blocking layer (not shown), and a buffer layer (not shown).

The hole transport region may include the hole injection layer 130 only or the hole transport layer 140 only. In some embodiments, the hole transport region may include a hole injection layer and a hole transport layer which are sequentially stacked on the first electrode 120. In some embodiments, the hole transport region may include a hole injection layer, a hole transport layer, and an electron blocking layer, which are sequentially stacked on the first electrode 11.

The hole injection layer 130 may include, for example, at least one selected from poly(ether ketone)-containing triphenylamine (TPAPEK), 4-iso-propyl-4′-methyl diphenyl iodonium tetrakis (pentafluorophenyl) borate (PPBI), N,N′-diphenyl-N,N′-bis-[4-(phenyl-m-tolyl-amino)-phenyl]-biphenyl-4,4′-diamine (DNTPD), copper phthalocyanine, 4,4′,4″-tris(3-methylphenyl phenyl amino) triphenylamine (m-MTDATA), N,N′-di(1-naphthyl)-N,N′-diphenylbenzidine (NPB), 4,4′,4″-tris (diphenyl amino) triphenylamine (TDATA), 4,4′,4″-tris(N,N-2-naphthyl phenyl amino) triphenylamine (2-TNATA), polyaniline/dodecylbenzene sulfonic acid (PANI/DBSA), poly(3,4-ethylenedioxythiophene)/poly(4-styrene sulfonate (PEDOT/PSS), polyaniline/10-camphor sulfonic acid (PANI/CSA), and polyaniline/poly(4-styrene sulfonate (PANI/PSS).

The hole injection layer 130 may be formed to a thickness of about 10 nanometers (nm) to about 1,000 nm, for example, about to a thickness of about 10 nm to about 100 nm.

The hole transport layer may include, for example, at least one selected from carbazole derivatives, e.g., 1,1-bis[(di-4-tolylamino)phenyl] cyclohexane (TAPC), N-phenylcarbazole, and polyvinylcarbazole, N,N′-bis(3-methylphenyl)-N,N′-diphenyl-[1,1-biphenyl]-4,4′-diamine (TPD), 4,4′,4″-tris(N-carbazolyl) triphenylamine (TCTA), N,N′-di(1-naphthyl)-N,N′-diphenylbenzidine (NPB), and poly(9,9-dioctyl-fluorene-co-N-(4-butylphenyl)-diphenylamine (TFB).

In some embodiments, the hole transport layer 140 may include at least one selected from Compound P-1, an amine group-containing polymer similar with Compound P-1, and Compound FA-14:

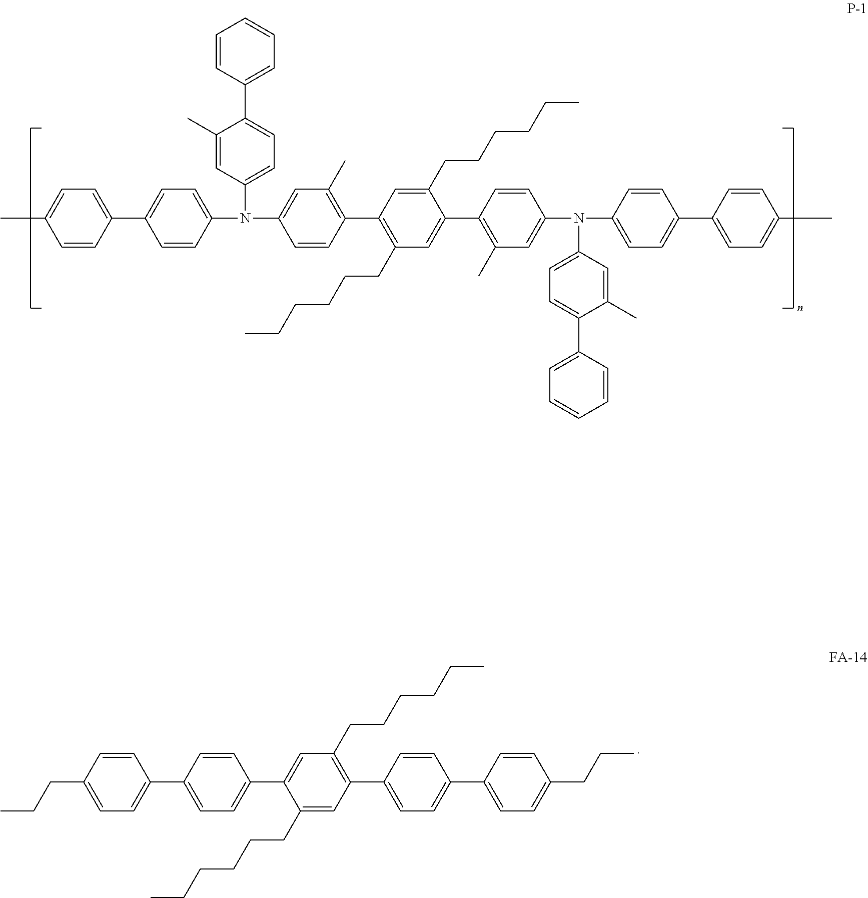

The hole transport layer 140 may be formed to a thickness of about 10 nm to about 1,000 nm, for example, about to a thickness of about 10 nm to about 150 nm.

The hole transport region may include a charge generating material as well as the aforementioned materials, to improve conductive properties of the hole transport region. The charge generating material may be substantially homogeneously or non-homogeneously dispersed in the hole transport region.

The charge generating material may include, for example, a p-dopant. The p-dopant may include one of a quinone derivative, a metal oxide, and a compound containing a cyano group, but embodiments are not limited thereto. For example, non-limiting examples of the p-dopant include a quinone derivative, such as tetracyanoquinodimethane (TCNQ) or 2,3,5,6-tetrafluoro-tetracyano-1,4-benzoquinonedimethane (F4-TCNQ); a metal oxide, such as a tungsten oxide or a molybdenum oxide; and a compound containing a cyano group, such as Compound HT-D1 or Compound HT-D2, but embodiments are not limited thereto:

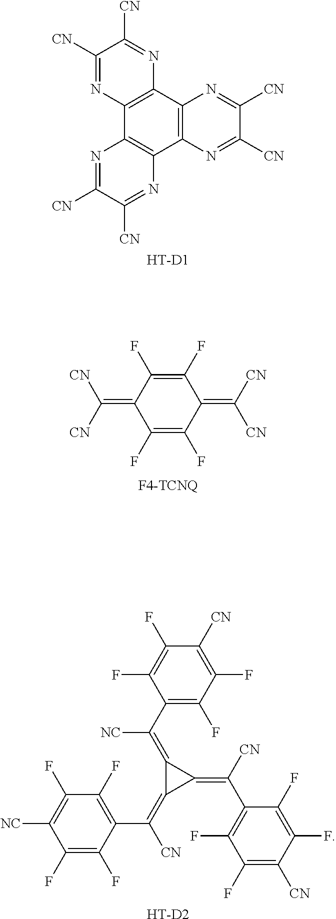

When the hole transport region includes a buffer layer, a material for forming the buffer layer may be selected from the materials for forming a hole transport region and host materials described herein, but embodiments are not limited thereto.

When the hole transport region includes an electron blocking layer, a material for forming the electron blocking layer may be selected from the materials for forming a hole transport region and host materials described herein, but embodiments are not limited thereto. In some embodiments, when the hole transport region includes an electron blocking layer, mCP may be used for forming the electron blocking layer.

The emission layer 150 may be formed on the hole transport region. The emission layer 150 may emit light by fluorescence or phosphorescence. The emission layer 150 may include a host and/or a dopant, and the host may include the heterocyclic compound represented by Formula 1. The emission layer 150 may include a known host material and a known dopant material.

In some embodiments, the host may include tris(8-quinolinato)aluminum (Alq₃), 4,4′-bis(carbazol-9-yl)biphenyl (CBP), poly(n-vinylcarbazole (PVK), 9,10-di(naphthaleneyl)anthracene (ADN), 4,4′,4″-tris(N-carbazolyl)triphenylamine (TCTA), 1,3,5-tris(N-phenyl-benzimidazol-2-yl)benzene (TPBi) 3-tert-butyl-9,10-di(naphth-2-yl)anthracene (TBADN), distyrylarylene (DSA), and 4,4′-bis(9-carbazole)-2,2′-dimethyl-biphenyl (dmCBP), but embodiments are not limited thereto.

In some embodiments, the dopant may include perylene and a derivative thereof, rubrene and a derivative thereof, coumarin and a derivative thereof, 4-dicyanomethylene-2-(p-dimethylaminostyryl)-6-methyl-4H-pyran (DCM) and a derivative thereof, an iridium complex, e.g., bis[2-(4,6-difluorophenyl)pyridinate] picolinate iridium (III) (Flrpic), bis(1-phenylisoquinoline)(acetylacetonate) iridium (III) (Ir(piq)₂(acac)), tris(2-phenylpyridine) iridium (III) (Ir(ppy)₃), tris(2-(3-p-xylyl)phenyl)pyridine iridium (III)) (dopant), and the like, an osmium complex, and a platinum complex, but embodiments are not limited thereto.

When the emission layer includes the host and the dopant, an amount of the dopant may be selected from a range of about 0.01 parts to about 15 parts by weight based on about 100 parts by weight of the host, but embodiments are not limited thereto.

The emission layer 150 may be formed to a thickness in a range of about 10 nm to about 60 nm.

When the organic light-emitting device 10 is a full-color organic light-emitting device, the emission layer may be patterned into a red emission layer, a green emission layer, and a blue emission layer. In some embodiments, the emission layer may have a structure in which the red emission layer, the green emission layer, and/or the blue emission layer are layered to emit white light. In some embodiments, the structure of the emission layer may vary.

Then, an electron transport region may be on the emission layer 150.

The electron transport region may include at least one selected from a hole blocking layer (not shown), the electron transport layer 160, and the electron injection layer 170.

In some embodiments, the electron transport region may have a hole blocking layer/an electron transport layer/an electron injection layer structure or an electron transport layer/an electron injection layer structure, but embodiments are not limited thereto. The electron transport layer may have a single-layered structure or a multi-layered structure including two or more different materials.

For example, in order to prevent diffusion of excitons or holes to the electron transport layer 160, the organic light-emitting device 100 may include a hole blocking layer between the electron transport layer 160 and the emission layer 150. The hole blocking layer may include, for example, at least one selected from an oxadiazole derivative, a triazole derivative, BCP, Bphen, and BAlq, but embodiments are not limited thereto:

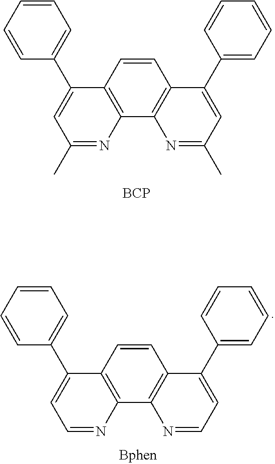

The thickness of the hole blocking layer may be in a range of about 20 Å to about 1,000 Å, and in some embodiments, about 30 Å to about 300 Å. While not wishing to be bound by theory, it is understood that when the thickness of the hole blocking layer is within any of these ranges, excellent hole blocking characteristics may be obtained without a substantial increase in driving voltage.

The electron transport layer 160 may include tris(8-quinolinato) aluminum (Alq₃); BAlq; a compound including pyridine ring, e.g., 1,3,5-tri[(3-pyridyl)-phen-3-yl]benzene); a compound including a triazine ring, e.g., 2,4,6-tris(3′-(pyridin-3-yl)biphenyl-3-yl)-1,3,5-triazine); a compound including an imidazole ring, e.g., 2-(4-(N-phenylbenzimidazolyl-1-yl-phenyl)-9,10-dinaphthylanthracene; a compound including a triazole ring, e.g., TAZ and NTAZ; 1,3,5-tris(N-phenyl-benzimidazol-2-Abenzene (TPBi), BCP, Bphen, and the like:

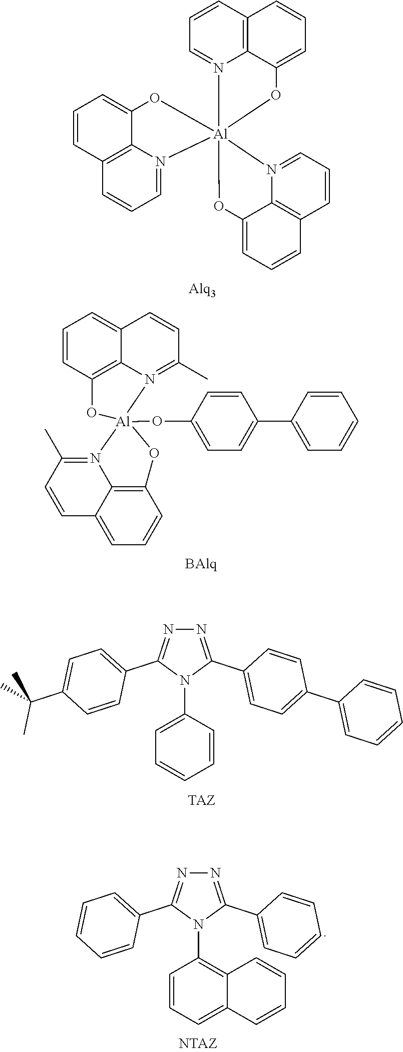

The electron transport layer 160 may include commercially available products such as KLET-01, KLET-02, KLET-03, KLET-10, or KLET-M1 (available from Chemipro Kasei).

The electron transport layer 160 may further include a material containing metal, in addition to the materials described above.

The material containing metal may include a Li complex. The Li complex may include, e.g., Compound ET-D1 (lithium 8-hydroxyquinolate, LiQ) or Compound ET-D2:

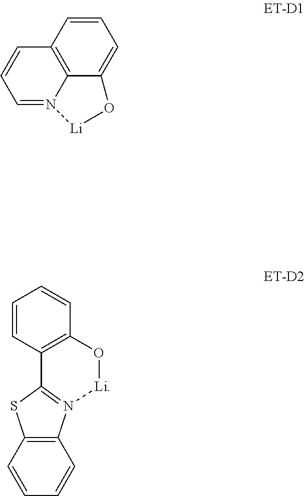

The electron transport layer 160 may be, for example, formed to a thickness in a range of about 15 nm to about 50 nm.

The electron injection layer 170 may be formed on the electron transport layer 160.

For example, the electron injection layer 170 may include a lithium compound, e.g., (8-hydroxyquinolinato)lithium (LiQ) and lithium fluoride (LiF), sodium chloride (NaCl), cesium fluoride (CsF), lithium oxide (Li₂O), or barium oxide (BaO).

In some embodiments, the electron injection layer 170 may be formed to a thickness in a range of about 0.3 nm to about 9 nm.

The second electrode 180 may be formed on the electron injection layer 170. The second electrode 180 may be a cathode, and be formed of a material with a relatively low work function selected from a metal, an alloy, an electrically conductive compound, a mixture thereof. For example, the second electrode 180 may be formed as a reflective electrode including a metal, e.g., lithium (Li), magnesium (Mg), aluminum (Al), or calcium (Ca), or an alloy, e.g., an aluminum-lithium (Al—Li) alloy, a magnesium-indium (Mg—In) alloy, or a magnesium-silver (Mg—Ag) alloy. In some embodiments, the second electrode 180 may be formed as a transparent electrode having a thickness of 20 nm or less and including a thin film of the metal or the alloy, or a transparent conductive film including indium tin oxide (n₂O₃—SnO₂) or indium zinc oxide (In₂O₃—ZnO).

Furthermore, a stacking structure of the organic light-emitting device 100 according to an embodiment is not limited to the foregoing description. The organic light-emitting device 100 according to an embodiment may have a different stacking structure known in the art. For example, the organic light-emitting device 100 may not include at least one selected from the hole injection layer 130, the hole transport layer 140, the electron transport layer 160, and the electron injection layer 170 or may further include another layer. In some embodiments, each layer of the organic light-emitting device 100 may be formed as a single layer or as multiple layers.

Methods of forming each layer of the organic light-emitting device 100 according to one or more embodiments are not particularly limited. For example, vacuum-deposition, solution coating, Langmuir-Blodgett (LB) deposition may be used in forming each layer thereof.

The solution coating may include spin coating, casting, micro-gravure coating, gravure coating, bar coating, roll coating, wire bar coating, dip coating, spray coating, screen printing, flexographic printing, offset printing, or ink-jet printing.

The solvent used in the solution coating may include toluene, xylene, diethyl ether, chloroform, ethyl acetate, dichloromethane, tetrahydrofuran, acetone, acetonitrile, N,N-dimethyl form amide, dimethyl sulfoxide, anisole, hexamethylphosphoric acid triamide, 1,2-dichloroethane, 1,1,2-trichloroethane, chlorobenzene, o-dichlorobenzene, dioxane, cyclohexane, n-pentane, n-hexane, n-heptane, n-octane, n-nonane, n-decane, methyl ethyl ketone, cyclohexanone, butyl acetate, ethyl cellosolve acetate, ethylene glycol, ethylene glycol monobutyl ether, ethylene glycol monoethyl ether, ethylene glycol monomethyl ether, dimethoxy ethane, propylene glycol, diethoxy methane, triethylene glycol monoethyl ether, glycerine, 1,2-hexanediol, methanol, ethanol, propanol, iso-propanol, cyclohexanol, and N-methyl-2-pyrrolidone. However, the solvent is not particularly limited. Any suitable solvent that may dissolve materials for forming each layer may be used.

In consideration of coatability or the like, a concentration of the composition may be about 0.1 percent by weight (weight %) or greater and 10 weight % or less, for example, about 0.5 weight % or greater and 5 weight % or less, but embodiments are not limited thereto.

The vacuum deposition may be performed at a deposition temperature in a range of about 100° C. to about 500° C., at a vacuum degree in a range of about 10⁻⁸ torr to about 10⁻³ torr, and at a deposition rate in a range of about 0.01 Angstroms per second (Å/sec) to about 100 Å/sec, though the conditions may vary depending on a compound that is used and a structure and thermal properties of a desired layer.

In some embodiments, the first electrode 120 may be an anode, and the second electrode 180 may be a cathode.

For example, the first electrode 120 may be an anode, the second electrode 180 may be a cathode, and an organic layer may include the emission layer 150 disposed between the first electrode 120 and the second electrode 180 and may further include a hole transport region disposed between the first electrode 120 and the emission layer 150 and an electron transport region disposed between the emission layer 150 and the second electrode 180, wherein the hole transport region may include at least one selected from the hole injection layer 130, the hole transport layer 140, a buffer layer, and an electron blocking layer, and the electron transport region may include at least one selected from a hole blocking layer, the electron transport layer 160, and the electron injection layer 170.

In some embodiments, the first electrode 120 may be a cathode, and the second electrode 180 may be an anode.

Hereinbefore the organic light-emitting device 10 has been described with reference to the FIGURE, but embodiments are not limited thereto.

General Definitions of Substituents

The term “X and Y may each independently” as used herein may refer to a case when X may be identical to or different from Y.

The term “substituted” as used herein refers to that a hydrogen atom in a substituent such as R₁₁ may be substituted with another substituent.

The term “C₁-C₆₀ alkyl group” as used herein refers to a linear or branched aliphatic hydrocarbon monovalent group having 1 to 60 carbon atoms. Examples of the C₁-C₆₀ alkyl group include a methyl group, an ethyl group, an n-propyl group, an iso-propyl group, an n-butyl group, an iso-butyl group, a sec-butyl group, a tert-butyl group, an n-pentyl group, an iso-pentyl group, a tert-pentyl group, a neopentyl group, a 1,2-dimethylpropyl group, an n-hexyl group, an iso-hexyl group, a 1,3-dimethylbutyl group, an 1-iso-propylpropyl group, a 1,2-dimethylbutyl group, an n-heptyl group, a 1,4-dimethylpentyl group, a 3-ethylpentyl group, a 2-methyl-1-iso-propylpropyl group, a 1-ethyl-3-methylbutyl group, an n-octyl group, a 2-ethylhexyl group, a 3-methyl-1-iso-propylbutyl group, a 2-methyl-1-iso-propyl group, a 1-tert-butyl-2-methylpropyl group, an n-nonyl group, a 3,5,5-trimethyldecyl group, an n-decyl group, an iso-decyl group, an n-undecyl group, a 1-methyldecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, an n-eicosyl group, an n-heneicosyl group, an n-docosyl group, an n-tricosyl group, and an n-tetracosyl group.

The term “C₁-C₆₀ alkylene group” as used herein refers to a divalent group having substantially the same structure as the C₁-C₆₀ alkyl group.

The term “C₁-C₆₀ alkoxy group” as used herein refers to a monovalent group represented by —OA₁₀₁ (wherein A₁₀₁ is a C₁-C₆₀ alkyl group). Examples of the C₁-C₆o alkoxy group include a methoxy group, an ethoxy group, a propoxy group, an iso-propoxy group, an n-butoxy group, an iso-butoxy group, a sec-butoxy group, a tert-butoxy group, an n-pentoxy group, an iso-pentoxy group, a tert-pentoxy group, a neopentoxy group, an n-hexyloxy group, an iso-hexyloxy group, a heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a 2-ethylhexyloxy group, and a 3-ethylpentyloxy group.

The term “C₁-C₆₀ alkylthio group” as used herein refers to a monovalent group represented by —SA₁₀₂ (wherein A₁₀₂ is a C₁-C₆₀ alkyl group).

The term “C₃-C₆₀ cycloalkyl group” as used herein refers to a monovalent monocyclic saturated hydrocarbon group including 3 to 10 ring-forming carbon atoms. Examples of the C₃-C₆₀ cycloalkyl group include a cyclopropyl group, a cyclobutyl group, a cyclopentyl group, a cyclohexyl group, and a cycloheptyl group. The term “C₃-C₆₀ cycloalkylene group” as used herein refers to a divalent group having substantially the same structure as the C₃-C₆₀ cycloalkyl group.

The term “C₆-C₆₀ aryl group” as used herein refers to a monovalent group having a carbocyclic aromatic system having 6 to 60 ring-forming carbon atoms (when the C₆-C₆₀ aryl group is substituted with a substituent, the carbon atom included in the substituent may not be counted as a ring-forming carbon atom). The term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₃₀ aryl group and the C₆-C₃₀ arylene group each independently include two or more rings, the respective rings may be fused.

The term “C₆-C₆₀ aryloxy group” as used herein refers to a group represented by —OA₁₀₃ (wherein A₁₀₃ is a C₆-C₆₀ aryl group). Examples of the C₆-C₆₀ aryloxy group include a 1-naphthyloxy group, a 2-naphthyloxy group, and a 2-azulenyloxy group.

The term “C₆-C₆₀ arylthio group” as used herein refers to a group represented by —SA₁₀₄ (wherein A₁₀₄ is a C₆-C₆₀ aryl group).

The term “C₁-C₆₀ heteroaryl group” as used herein refers to a monovalent group having a heterocyclic aromatic system having at least one heteroatom selected from N, O, Si, P, and S as a ring-forming atom and 1 to 60 ring-forming carbon atoms. The term “C₁-C₆₀ heteroarylene group” as used herein refers to a divalent group having a heterocyclic aromatic system having at least one heteroatom selected from N, O, Si, P, and S as a ring-forming atom and 1 to 60 ring-forming carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each independently include two or more rings, the respective rings may be fused.

The term “C₁-C₆₀ heteroaryloxy group” as used herein refers to a group represented by —oA₁₀₅ (wherein A₁₀₅ is a C₁-C₆₀ heteroaryl group). Examples of the C₁-C₆₀ heteroaryloxy group include a 2-furanyloxy group, a 2-thienyloxy group, a 2-indolyloxy group, a 3-indolyloxy group, a 2-benzofuryloxy group, and a 2-benzothienyloxy group.

The term “C₁-C₆₀ heteroarylthio group” as used herein refers to a group represented by —SA₁₀₆ (wherein A₁₀₆ is a C₁-C₆₀ heteroaryl group).

The term “C₇-C₃₀ arylalkyl group” as used herein refers to a monovalent group in which an alkyl group is substituted with an aryl group. The total number of carbon atoms forming the alkyl group and the aryl group may be in a range of 7 to 30. Examples of the C₇-C₃₀ aryl alkyl group include a benzyl group, a phenylethyl group, a phenylpropyl group, and a naphthylmethyl group.

The term “C₆-C₃₀ arylalkyloxy group” as used herein refers to a group represented by —OA₁₀₅ (wherein A₁₀₅ is a C₇-C₃₀ arylalkyl group).

The term “C₆-C₃₀ arylalkylthio group” as used herein refers to a group represented by —SA₁₀₆ (wherein A₁₀₆ is a C₇-C₃₀ arylalkyl group).

The term “C₈-C₃₀ arylalkenyl group” as used herein refers to a monovalent group in which an alkenyl group is substituted with an aryl group. The total number of carbon atoms forming the alkenyl group and the aryl group may be in a range of 8 to 30.

The term “C₈-C₃₀ arylalkynyl group” as used herein refers to a monovalent group in which an alkynyl group is substituted with an aryl group. The total number of carbon atoms forming the alkynyl group and the aryl group may be in a range of 8 to 30.

The term “monovalent non-aromatic condensed polycyclic group” as used herein refers to a monovalent group that has two or more condensed rings and only carbon atoms (e.g., the number of carbon atoms may be in a range of 8 to 60) as ring-forming atoms, wherein the molecular structure as a whole is non-aromatic. Examples of the monovalent non-aromatic condensed polycyclic group include a fluorenyl group. The term “divalent non-aromatic condensed polycyclic group” as used herein refers to a divalent group having substantially the same structure as the monovalent non-aromatic condensed polycyclic group.

The term “monovalent non-aromatic condensed heteropolycyclic group” as used herein refers to a monovalent group that has two or more condensed rings, and a heteroatom selected from N, O, P, Si, and S and carbon atoms (e.g., the number of carbon atoms may be in a range of 1 to 60) as ring-forming atoms, wherein the molecular structure as a whole is non-aromatic. Examples of the monovalent non-aromatic condensed heteropolycyclic group include a carbazolyl group. The term “divalent non-aromatic condensed heteropolycyclic group” as used herein refers to a divalent group having substantially the same structure as the monovalent non-aromatic condensed heteropolycyclic group.

The term “C₅-C₆₀ carbocyclic group” as used herein refers to a saturated or unsaturated cyclic group including 5 to 60 carbon atoms only as ring-forming atoms. The C₅-C₆₀ carbocyclic group may be a monocyclic group or a polycyclic group. Depending on formula structure, the C₅-C₃₀ carbocyclic group may be monovalent, divalent, trivalent, quadrivalent, pentavalent, or hexavalent.

The term “C₁-C₆₀ heterocyclic group” as used herein refers to saturated or unsaturated cyclic group including 1 to 60 carbon atoms and at least one heteroatom selected from N, O, P, Si, and S as ring-forming atoms. The C₁-C₆₀ heterocyclic group may be a monocyclic group or a polycyclic group. Depending on a formula structure, the C₅-C₃₀ heterocyclic group may be monovalent, divalent, trivalent, quadrivalent, pentavalent, or hexavalent.

In the present specification, at least one substituent of the substituted C₅-C₆₀ carbocyclic group, the substituted C₁-C₆₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂⁻C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₁₃)(Q₁₄)(Q₁₅), —B(Q₁₆)(Q₁₇), and —P(═O)(Q₁₈)(Q₁₉);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), and —P(═O)(Q₂₈)(Q₂₉), and

—N(Q₃₁)(Q₃₂), —Si(Q₃₃)(Q₃₄)(Q₃₅), —B(Q₃₆)(Q₃₇), and —P(=O)(Q₃₈)(Q₃₉),

wherein Q₁ to Q₉, Q₁₁ to Q₁₉, Q₂₁ to Q₂₉, and Q₃₁ to Q₃₉ may each independently be selected from hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-Cio heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one selected from a C₁-C₆₀ alkyl group and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group.

The term “A to B” as used herein refers to a range from A to B including A and B.

Hereinafter, with reference to Examples and Comparative Examples, the heterocyclic compound represented by Formula 1 and an organic light-emitting device including the heterocyclic compound will be further described. However, these Examples are illustrative purposes only, and thus, the heterocyclic compound and the organic light-emitting device according to an embodiment is not limited to the following Examples.

The wording “B was used instead of A” used in describing Synthesis Examples means that an identical molar equivalent of B was used in place of A.

Also, unless otherwise described, “%” is based on a weight.

### EXAMPLES

### Synthesis Example 1

### Synthesis of Compound 1

Compound 1 was synthesized according to Reaction Scheme 1: Intermediate E was synthesized according to Reaction Scheme 2.

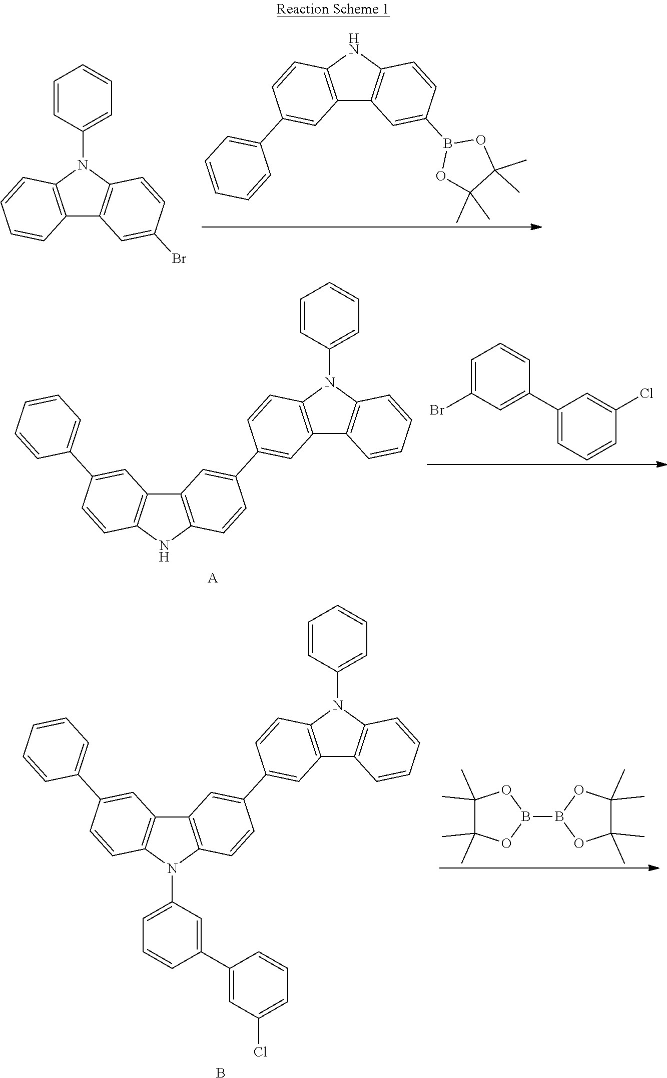

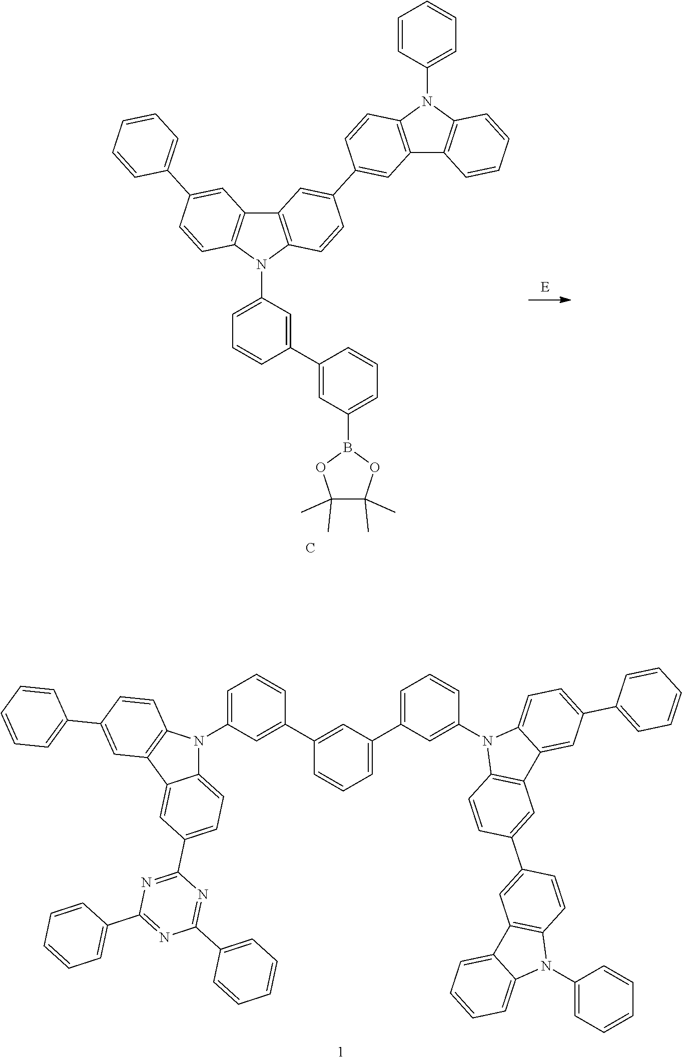

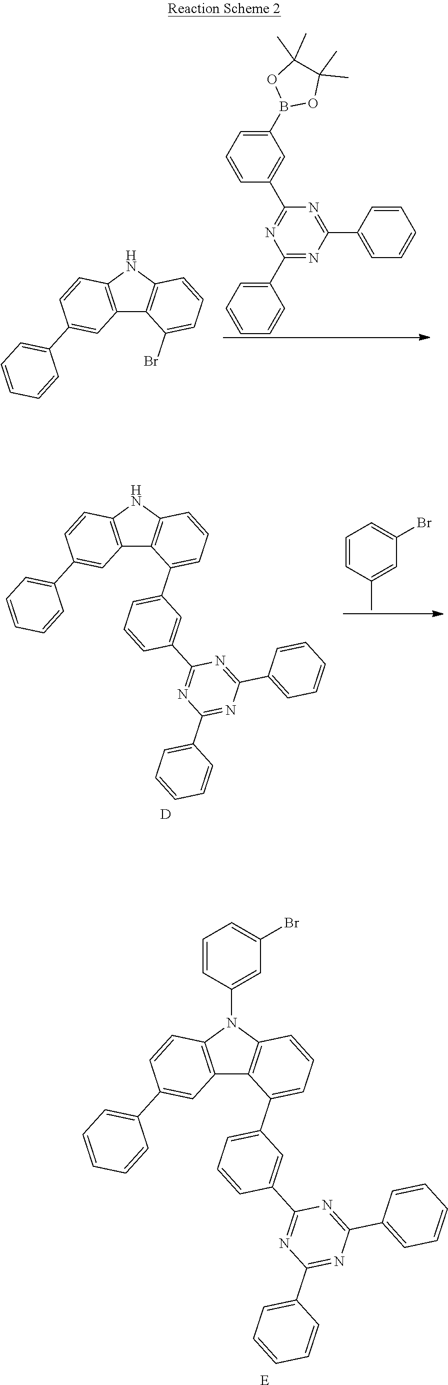

(1) Synthesis of Intermediate A

15.00 grams (g, 46.55 mmol) of 3-bromo-9-phenyl-9H-carbazole, 17.19 g (46.55 mmol) of 3-phenyl-6-(4,4,5,5-tetramethyl-1,3,2-dioxabororane-2-yl)-9H-carbazole, 1.61 g (1.40 mmol) of tetrakis(triphenylphosphine)palladium(0), 137 milliliters (mL) of toluene, 51 mL of ethanol, and 137 mL of 2 molar (M) sodium carbonate aqueous solution were mixed together. Under nitrogen atmosphere, the mixture was stirred at a temperature of 90° C. for 3 hours. Once the reaction was complete, an extraction process was performed using toluene/water, and then a solvent of an organic layer was distilled. Subsequently, Intermediate A was obtained by purifying through a column chromatography.

(2) Synthesis of Intermediate B

10.00 g (20.64 mmol) of Intermediate A, 6.07 g (46.55 mmol) of 3-bromo-3′-chlorophenyl, 0.37 g (1.65 mmol) of palladium acetate (II), 0.96 g (3.30 mmol) of tri-tert-butylphosphonium tetrafluoroborate, 2.98 g (30.95 mmol) of sodium tert-butoxide, and 100 mL of toluene were mixed together. Under nitrogen atmosphere, the mixture was stirred at a temperature of 120° C. for 4 hours. Once the reaction was complete, an extraction process was performed using toluene/water, and then a solvent of an organic layer was distilled. Subsequently, Intermediate B was obtained by purifying through a column chromatography.

(3) Synthesis of Intermediate C

8.00 g (11.92 mmol) of Intermediate B, 4.54 g (17.88 mmol) of bis(pinacolato)diborane, 0.55 g (0.6 mmol) of tris(dibenzylideneacetone)dipalladium (0), 0.57 g (1.19 mmol) of XPhos, 5.26 g (53.63 mmol) of potassium acetate, and 120 mL of 1,4-dioxane were mixed together. Under nitrogen atmosphere, the mixture was stirred at a temperature of 100° C. for 4 hours. Once the reaction was complete, a solvent was distilled to thereby obtain Intermediate C.

(4) Synthesis of Intermediate D

Under nitrogen atmosphere, 18.30 g (56.8 mmol) of 5-bromo-3-phenyl-9H-carbazole, 24.52 g (54.1 mmol) of 2,4-diphenyl-6-(3-(4,4,5,5-tetramethyl-1,3,2-dioxaborolan-2-yl)phenyl)-1,3,5-triazine, 0.949 g (1.35 mmol) of dichlorobis (triphenylphosphine)dipalladium, 154 mL of dioxane, 77 mL of toluene, and 148 mL of 1 M sodium carbonate aqueous solution were added to a reaction vessel. The mixture was stirred at a temperature of 85° C. for 6 hours. Once the reaction was complete, the mixture was cooled to room temperature, and an extraction process was performed using toluene/water, followed by washing with chloroform/hexane, thereby obtaining Intermediate D.

(5) Synthesis of Intermediate E

Under argon atmosphere, 26.90 g (48.9 mmol) of Intermediate D, 14.51 g (51.3 mmol) of 1-bromo-3-iodine benzene, 1.86 g (9.77 mmol) of iodized copper (I), 3.35 g (29.3 mmol) of trans-1,2-cyclohexanediamine, 9.39 g (97.7 mmol) of sodium tert-butoxide, and 242 mL of dioxane were added to a reaction vessel. Then, the mixture was stirred at a temperature of 100° C. for 7 hours. Once the reaction was complete, the reaction mixture was cooled to room temperature. Then, using a Celite filter, impurities were filtered and separated. A solvent was distilled therefrom, and purification was performed using column chromatography, thereby obtaining Intermediate E.

(6) Synthesis of Compound 1

8.00 g (10.49 mmol) of Intermediate C, 7.40 g (10.49 mmol) of Intermediate E, 0.49 g (0.52 mmol) of tris(dibenzylideneacetone)dipalladium (0), 0.50 g (1.05 mmol) of XPhos, 127 mL of toluene, 32 mL of ethanol, and 13 mL of 2 M sodium carbonate aqueous solution were mixed together. Then, the mixture was stirred at a temperature of 90° C. for 4 hours under nitrogen atmosphere. Once the reaction was complete, an extraction process was performed using toluene/water, and then a solvent of an organic layer was distilled. Subsequently, Compound 1 was obtained by purifying through a column chromatography. The molecular weight of Compound 1 was 1261.54.

### Synthesis Example 2

### Synthesis of Materials for Hole Transport Layer

According to the method disclosed in WO 2011-159872, Polymer P-1 corresponding to Compound T was synthesized. The formula of P-1 is shown below. Regarding the molecular weight of P-1, Mn=141,000, and Mw=511,000.

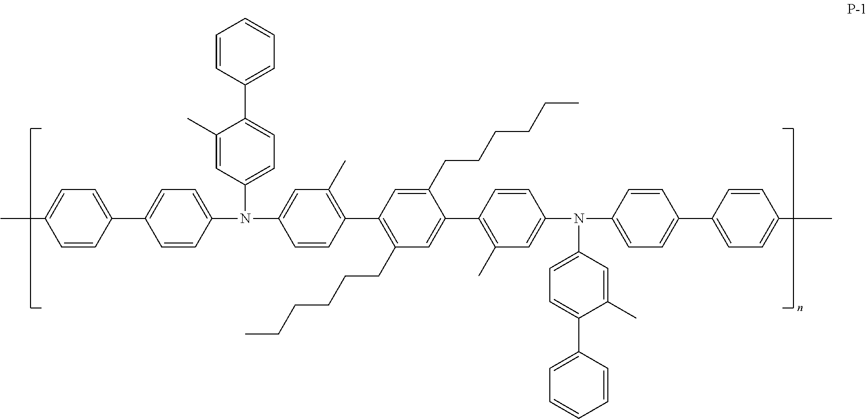

FA-14 disclosed in US 2016/0315259 was synthesized. The formula of FA-14 is shown below.

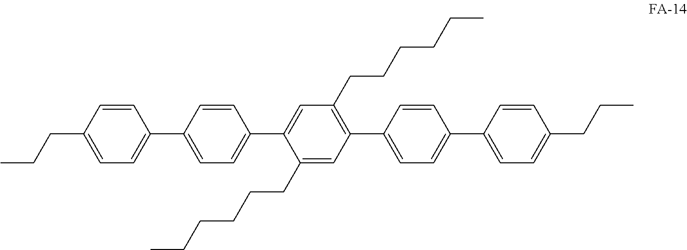 [SMILES: CCCCCCc1cc(-c2ccc(-c3ccc(CCC)cc3)cc2)c(CCCCCC)cc1-c1ccc(-c2ccc(CCC)cc2)cc1]

### Example 1

PEDOT-PSS (available from Sigma-Aldrich) was spin-coated on a glass substrate having 150 nm stripe-patterned ITO (indium tin oxide, anode) to form a dried film having a thickness of 15 nm, thereby forming a hole injection layer.

A coating liquid, in which P-1 and FA-14 were dissolved in anisole, for forming a hole transport layer (wherein the content of FA-14 is 20 weight % based on the total content of the hole transport layer) was spin-coated on the hole injection layer. The result was heated at a temperature of 250° C. for 1 hour to thereby form a hole transport layer having a dried film having a thickness of 125 nm.

Also, 4 weight % of a coating liquid, in which Compound 1 as a host material on the hole transport layer and tris(2-(3-p-xylyl)phenyl)pyridine iridium (III) as a dopant material were dissolved in methyl benzoate, for forming an emission layer was spin-coated on the hole transport layer to form an emission layer (wherein a doped weight of the dopant material was 10 weight % based on the total weight of the emission layer) to a thickness of 55 nm as a dried film.

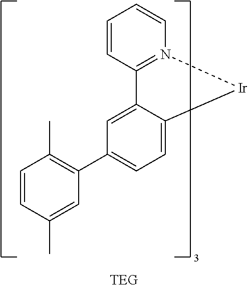

Lithium 8-hydroxyquinolate (hereinafter, referred to as LiQ) and KLET-03 (available from Chemipro Kasei Co., Ltd.) were co-deposited on the emission layer at a ratio of 1:1 using a vacuum deposition apparatus to form an electron transport layer having a thickness of 20 nm. Lithium fluoride (hereinafter, referred to as LiF) was deposited on the electron transport layer using a vacuum deposition apparatus to form an electron injection layer having a thickness of 3.5 nm. Further, aluminum was deposited on the electron injection layer using a vacuum deposition apparatus to form a second electrode (cathode) having a thickness of 100 nm, thereby completing the manufacture of an organic light-emitting device.

### Examples 2 to 4 and Comparative Examples 1 and 2

Organic light-emitting devices were manufactured in substantially the same manner as in Example 1, except that compounds shown in Table 1 were used as a host material in each emission layer.

However, in Comparative Example 1 the host material was not fully dissolved in a solvent, and thus, an emission layer was not formed, thus resulting in failure of the manufacture of an organic light-emitting device.

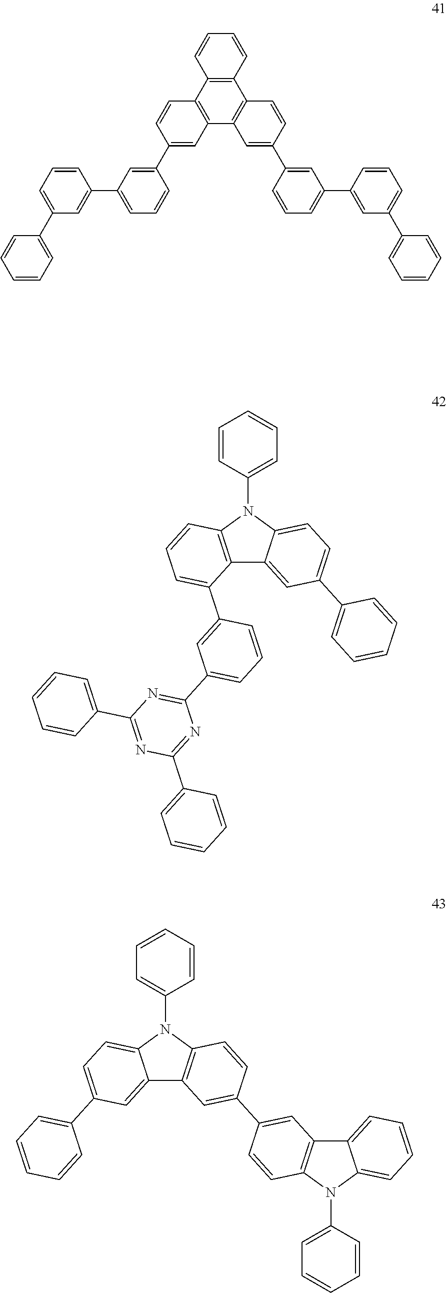

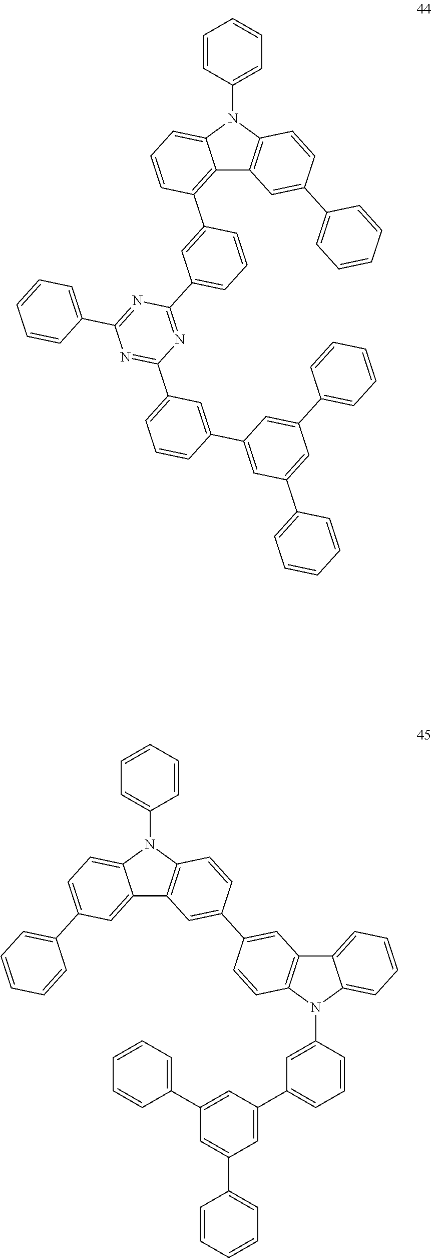 [SMILES: c1ccc(-c2cc(-c3ccccc3)cc(-c3cccc(-c4nc(-c5ccccc5)nc(-c5cccc(-c6cccc7c6c6cc(-c8ccccc8)ccc6n7-c6ccccc6)c5)n4)c3)c2)cc1 ; c1ccc(-c2cc(-c3ccccc3)cc(-c3cccc(-n4c5ccccc5c5cc(-c6ccc7c(c6)c6cc(-c8ccccc8)ccc6n7-c6ccccc6)ccc54)c3)c2)cc1]

### Evaluation Example 1

The current efficiency and luminescence efficiency of the organic light-emitting devices manufactured in Examples 1 to 4 and Comparative Examples 1 and 2 were evaluated in the following manner.

A predetermined voltage was applied to each of the organic light-emitting devices by using a direct-current constant-voltage power source (for example, a source meter available from KEYENCE Co., Ltd.) for the emission of the organic light-emitting device. The emission of each of the organic light-emitting device was measured by a luminance meter (for example, SR-3 available from Topcom Co., Ltd) while a current applied to the organic light-emitting device was gradually raised. Then, when the luminance reached 6,000 candelas per square meter (cd/m²), the current was set to be constant, and then the organic light-emitting device was standed for.

Then, a value of current per unit area (current density) of the organic light-emitting device was calculated, and the luminance (cd/m²) was divided by the current density (amperes per square meter, A/m²) to calculate “current efficiency (candelas per ampere, cd/A)”.

In addition, the “emission lifespan (LT₉₅, hour)” indicates time (hour) for the luminance measured by the luminance meter to decline to 95% of its initial luminance.

The results of evaluation are shown in Table 1. The current efficiency and emission lifespan of Examples 1 to 4 and Comparative Examples 1 and 2 are shown in values relative to 100 of the measured values of Comparative Example 1.

TABLE 1

| | | Current | |
|---|---|---|---|
| | | efficiency | Emission |
| | | (relative | lifespan |
| | Host material | value) | (relative value) |
| Example 1 | Compound 1 | 125 | 450 |
| Example 2 | Compound 1:Compound 41 | 137 | 636 |
| | (1:1 weight ratio) | | |
| Example 3 | Compound 1:Compound 41 | 135 | 830 |
| | (2:3 weight ratio) | | |
| Example 4 | Compound 1:Compound 44 | 145 | 363 |
| Comparative | Compound 42:Compound 43 | Film | Film formation |
| Example 1 | (1:1 weight ratio) | formation | failed |
| | | failed | |
| Comparative | Compound 44:Compound 45 | 100 | 100 |
| Example 2 | (1:1 weight ratio) | | |

As shown in Table 1, the organic light-emitting devices of Example 1 to 4 were prepared by using a coating method, and the current efficiency and emission lifespan were found to be excellent.

Also, in Example 2, when a known material and the heterocyclic compound is used in combination, the current efficiency and emission lifespan were found to be excellent. In Example 3, by adjusting the mixed ratio with a known material, an organic light-emitting device having a longer lifespan was obtained.

Further, referring to Example 4, when an electron transporting host and the heterocyclic compound are used in combination, the current efficiency and emission lifespan are excellent. In particular, an organic light-emitting device that is further efficient was obtained.

In Comparative Example 1, as the host material was not fully dissolved in a solvent, the emission layer was not formed. Therefore, in Comparative Example 1, an organic light-emitting device was not prepared. Thus, it was not possible to measure the current efficiency and emission lifespan.

As apparent from the foregoing description, the heterocyclic compound has excellent electrical characteristics and/or thermal stability. Accordingly, an organic light-emitting device employing the heterocyclic compound has an improved current efficiency and lifespan characteristics.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present description as defined by the following claims.

What is claimed is:

1. A heterocyclic compound represented by Formula 1: 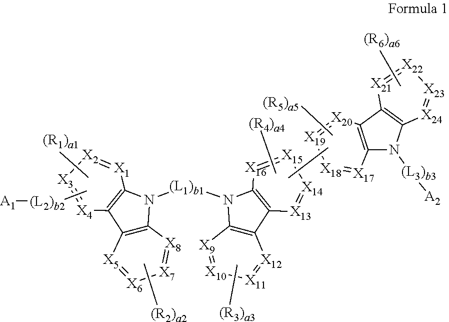
wherein, in Formula 1,
X₁ to X₂₄ are each independently selected from carbon (C) and nitrogen (N),
at least one selected from X₁ and X₄ is C and is bound to -(L₂)_{b2}-A1,
L₁ is selected from a substituted or unsubstituted C₁-C₂₀ alkylene group, a substituted or unsubstituted C₃-C₂₀ cycloalkylene group, a substituted or unsubstituted C₅-C₆₀ carbocyclic group, and a substituted or unsubstituted C₁-C₆₀ heterocyclic group,
L₂ and L₃ are each independently selected from a single bond, a substituted or unsubstituted C₁-C₂₀ alkylene group, a substituted or unsubstituted C₃-C₂₀ cycloalkylene group, a substituted or unsubstituted Cs-C₆₀ carbocyclic group, and a substituted or unsubstituted C₁-C₆₀ heterocyclic group,
b1 is an integer selected from 1 to 5,
b2 and b3 are each independently an integer from 0 to 5,
A₁ and A₂ are each independently selected from a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, and a substituted or unsubstituted C₅-C₆₀ heteroaryl group, provided that at least one selected from A₁ and A₂ is a C₅-C₆₀ heteroaryl group,
R₁ to R₆ are each independently selected from hydrogen, deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-Cio heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, and a monovalent non-aromatic condensed heteropolycyclic group,
a1 , a4, and a5 are each independently an integer from 0 to 3, and
a2, a3, and a6 are each independently an integer from 0 to 4.

2. The heterocyclic compound of claim 1, wherein a molecular weight of the heterocyclic compound is in a range of 1,000 Daltons to 3,000 Daltons.

3. The heterocyclic compound of claim 1, wherein at least one selected from L₁ to L₃ is selected from a substituted or unsubstituted C₆-C₆₀ carbocyclic group, a heterocyclic group comprising 5 to 60 ring-forming atoms and O or S as a heteroatom, and a substituted or unsubstituted C₃-C₆₀ cycloalkylene group.

4. The heterocyclic compound of claim 1, wherein L₁ to L₃ are each independently selected from the following structures:

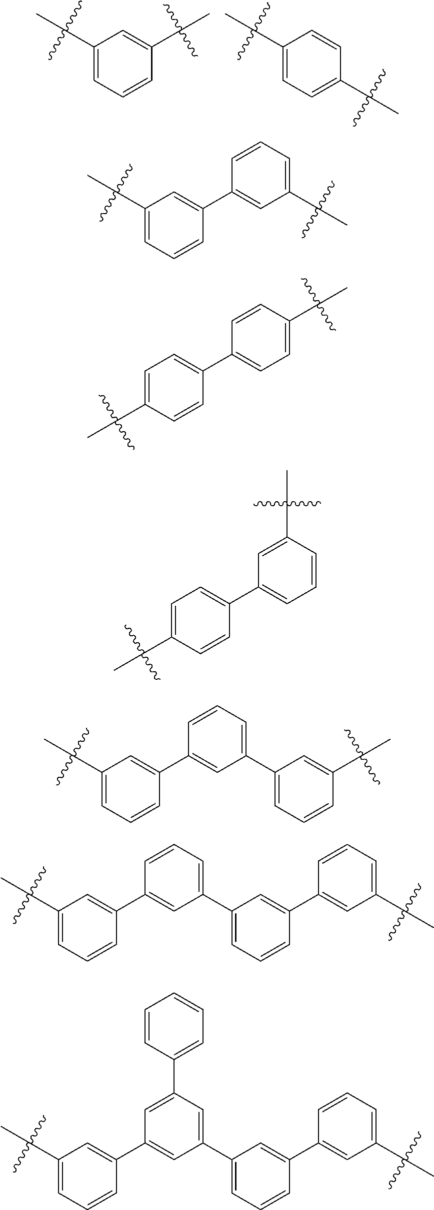

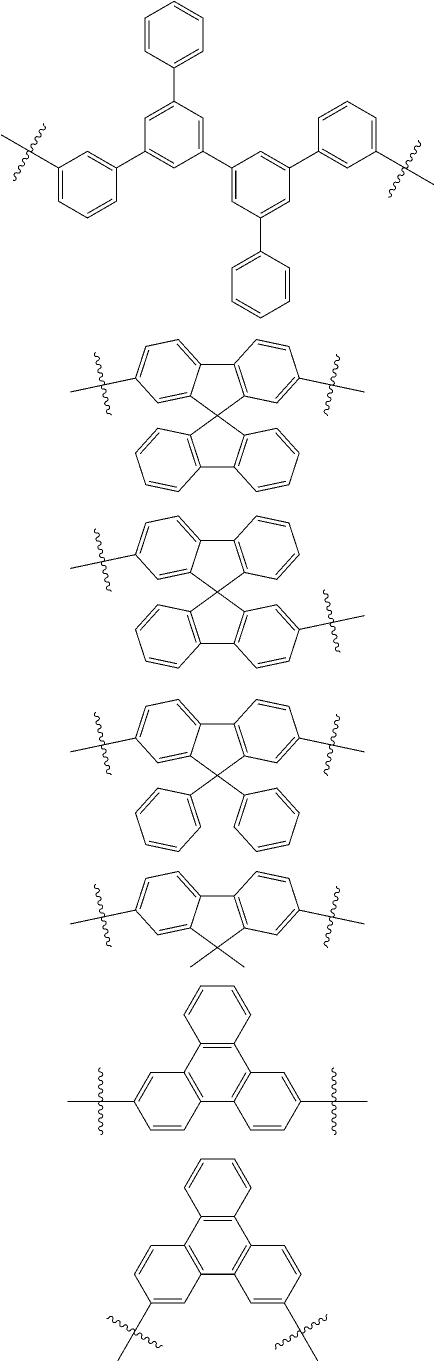

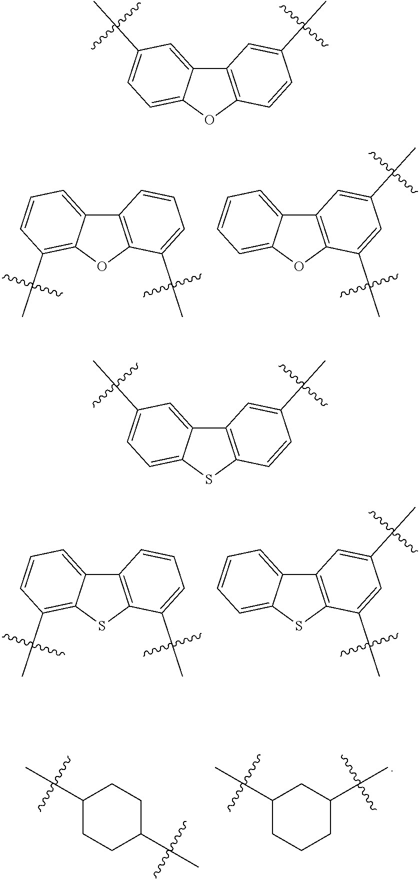

5. The heterocyclic compound of claim 1, wherein at least one selected from A₁ and A₂ is a substituted or unsubstituted C₁-C₆₀ heterocyclic group comprising nitrogen.

6. The heterocyclic compound of claim 1, wherein at least one selected from A₁ and A₂ is selected from a substituted or unsubstituted pyridinyl group, a substituted or unsubstituted pyrazinyl group, a substituted or unsubstituted pyrimidinyl group, a substituted or unsubstituted pyridazinyl group, a substituted or unsubstituted triazinyl group, a substituted or unsubstituted azadibenzofuranyl group, a substituted or unsubstituted diazabenzofuranyl group, a substituted or unsubstituted azadibenzothiophenyl group, and a substituted or unsubstituted diazabenzothiophenyl group.

7. The heterocyclic compound of claim 1, wherein at least one selected from A₁ and A₂ is selected from Formulae 5-1 to 5-25:

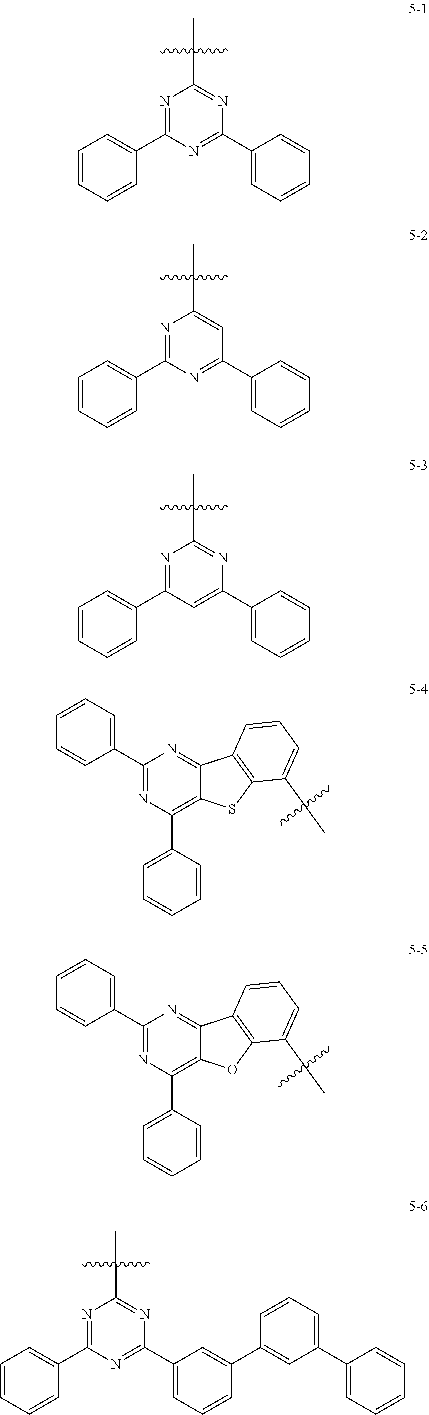 [SMILES: *c1nc(-c2ccccc2)nc(-c2ccccc2)n1 ; *c1cc(-c2ccccc2)nc(-c2ccccc2)n1 ; *c1nc(-c2ccccc2)cc(-c2ccccc2)n1 ; *c1cccc2c1sc1c(-c3ccccc3)nc(-c3ccccc3)nc12 ; *c1cccc2c1oc1c(-c3ccccc3)nc(-c3ccccc3)nc12 ; *c1nc(-c2ccccc2)nc(-c2cccc(-c3cccc(-c4ccccc4)c3)c2)n1]

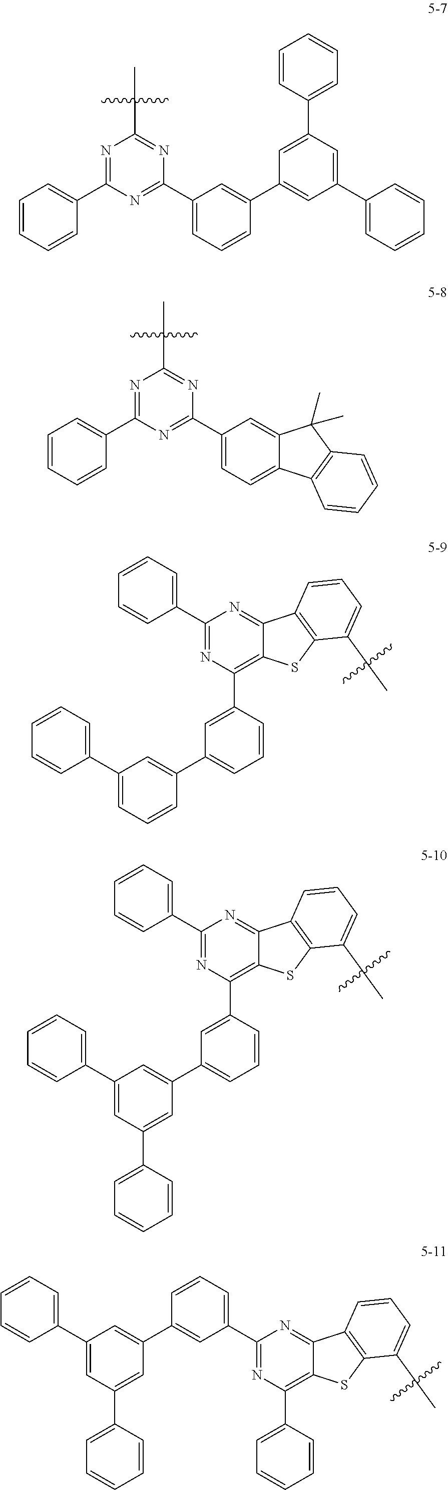

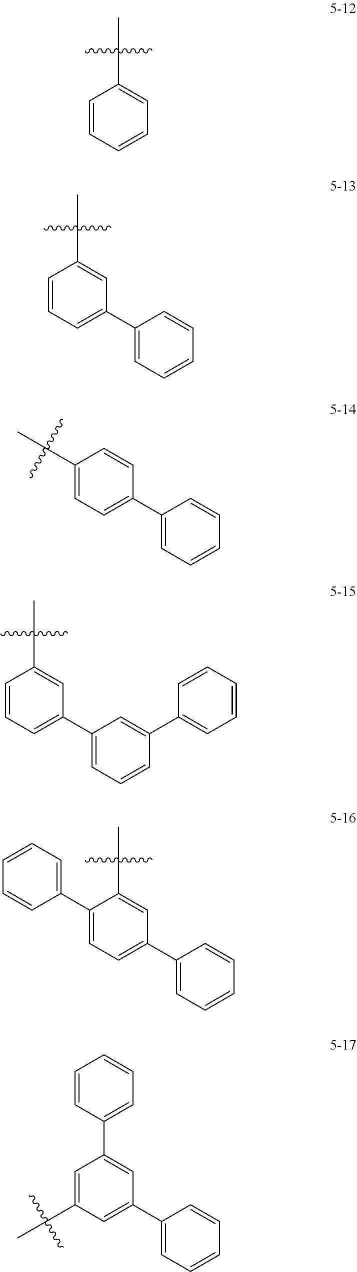 [SMILES: *c1ccccc1 ; *c1cccc(-c2ccccc2)c1 ; *c1ccc(-c2ccccc2)cc1 ; *c1cccc(-c2cccc(-c3ccccc3)c2)c1 ; *c1cc(-c2ccccc2)ccc1-c1ccccc1 ; *c1cc(-c2ccccc2)cc(-c2ccccc2)c1]

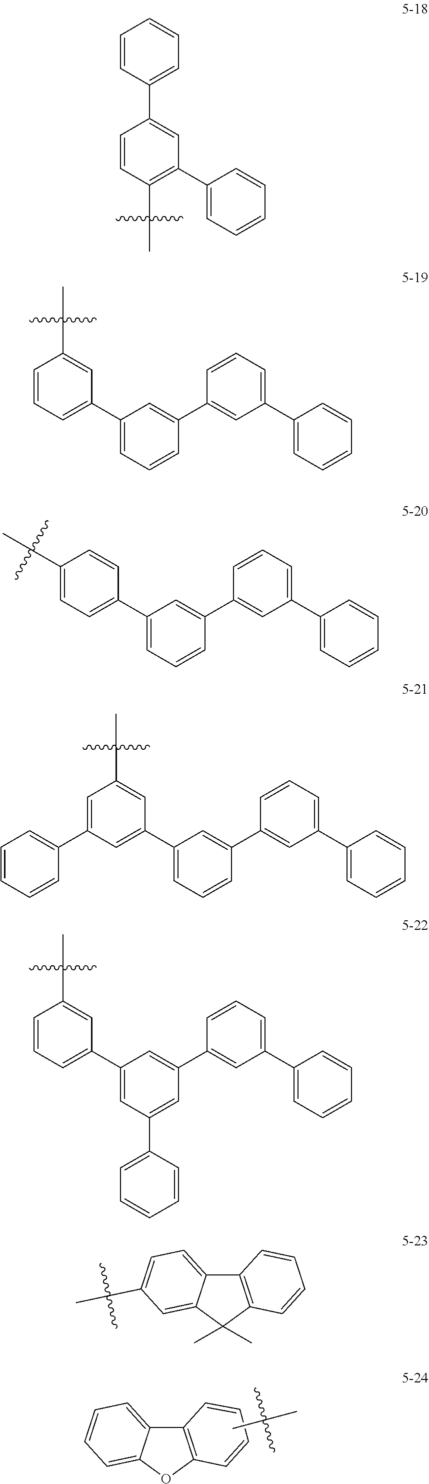

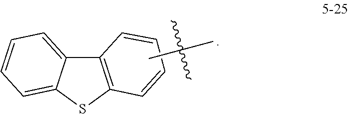

8. The heterocyclic compound of claim 7, wherein A₁ is selected from Formulae 5-1 to 5-11.

9. The heterocyclic compound of claim 1, wherein R₁ to R₆ are each independently hydrogen, —F, —Cl, —Br, —I, a substituted or unsubstituted C₁-C₂₀ alkyl group, a substituted or unsubstituted C₃-C₆₀ cycloalkyl group, a substituted or unsubstituted C₃-C₆₀ heterocycloalkyl group, a substituted or unsubstituted C₆-C₆₀ aryl group, a C₁-C₆₀ heteroaryl group, a monovalent non-aromatic condensed polycyclic group, and a monovalent non-aromatic condensed heteropolycyclic group.

10. The heterocyclic compound of claim 1, wherein the heterocyclic compound represented by Formula 1 is represented by Formula 10: 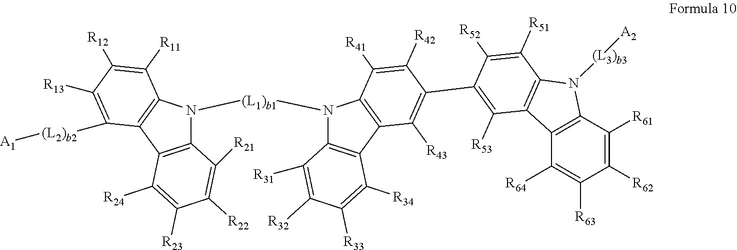
wherein, in Formula 10,
L₁ to L₃, A₁, A₂, and b₁ to b₃ are each defined the same as in claim 1,
R₁₁ to R₁₃ are each defined the same as R₁ in claims 1,
R₂₁ to R₂₄ are each defined the same as R₂ in claims 1,
R₃₁ to R₃₄ are each defined the same as R₃ in claims 1,
R₄₁ to R₄₃ are each defined the same as R₄ in claims 1,
R₅₁ to R₅₃ are each defined the same as R₅ in claims 1, and
R₆₁ to R₆₄ are each defined the same as R₆ in claim 1.

11. The heterocyclic compound of claim 10, wherein R₁₁ to R₁₃, R₂₁, R₂₂, R₂₄, R₃₁, R₃₂, R₃₄, R₄₁ to R₄₃, R₅₁ to R₅₃, and R₆₁ to R₆₄ are each hydrogen,
R₂₃ is selected from hydrogen, a cyclopentyl group, a cyclohexyl group, a cycloheptyl group, a phenyl group, a naphthyl group, an anthracenyl group, a phenanthrenyl group, a pyrenyl group, a chrysenyl group, a triphenylenyl group, a fluorenyl group, a pyridinyl group, a pyrimidinyl group, a pyrazinyl group, a pyridazinyl group, a triazinyl group, a quinolinyl group, a quinazolinyl group, a quinoxalinyl group, an isoquinolinyl group, a carbazolyl group, a dibenzofuranyl group, a dibenzothiophenyl group, a biphenyl group, and a terphenyl group, and
R₃₃ is selected from a cyclopentyl group, a cyclohexyl group, a cycloheptyl group, a phenyl group, a naphthyl group, an anthracenyl group, a phenanthrenyl group, a pyrenyl group, a chrysenyl group, a triphenylenyl group, a fluorenyl group, a pyridinyl group, a pyrimidinyl group, a pyrazinyl group, a pyridazinyl group, a triazinyl group, a quinolinyl group, a quinazolinyl group, a quinoxalinyl group, an isoquinolinyl group, a carbazolyl group, a dibenzofuranyl group, a dibenzothiophenyl group, a biphenyl group, and a terphenyl group.

12. The heterocyclic compound of claim 1, wherein the heterocyclic compound represented by Formula 1 is asymmetric.

13. The heterocyclic compound of claim 1, wherein the heterocyclic compound represented by Formula 1 is selected from Compounds 1 to 36:

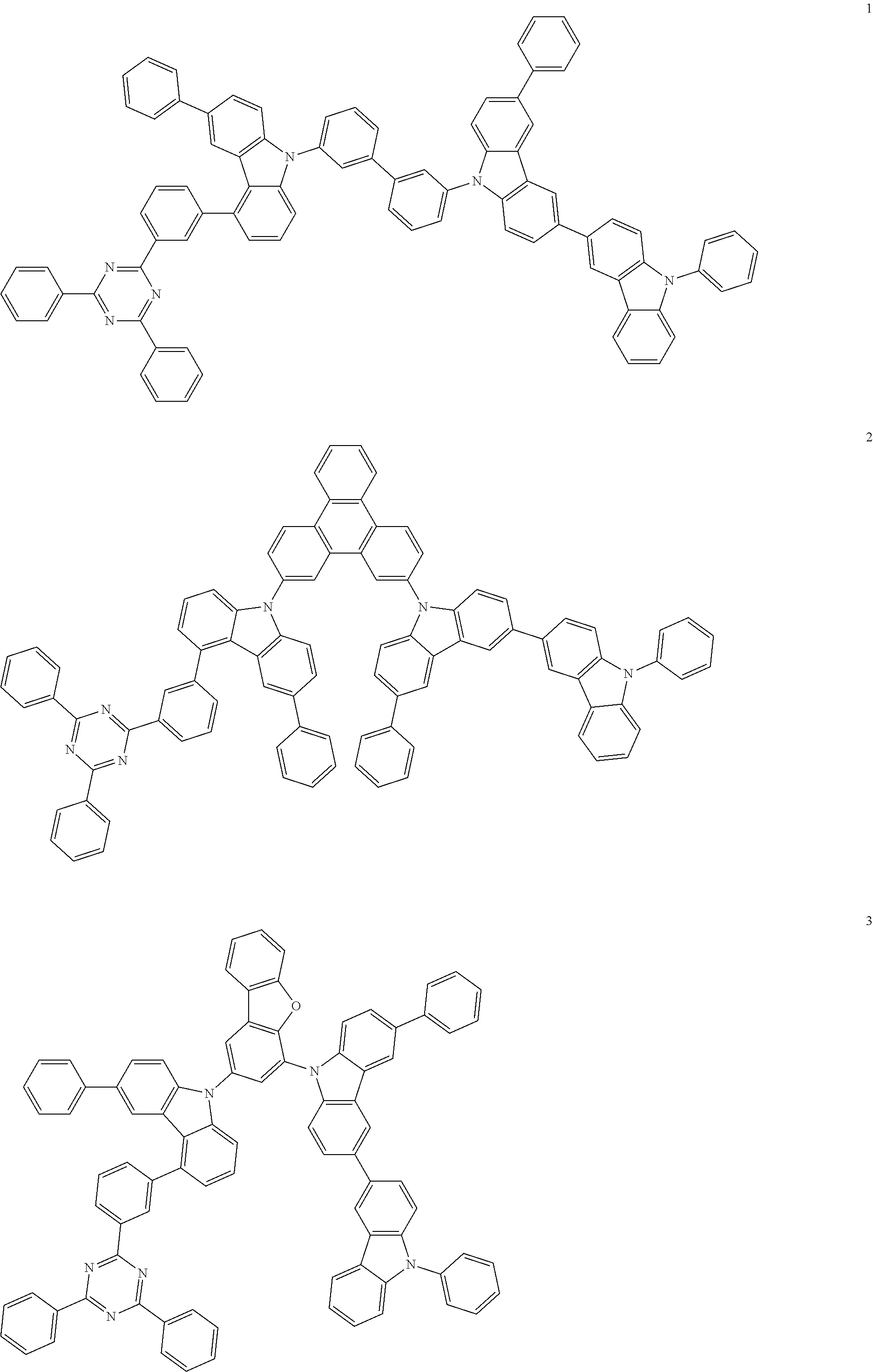 [SMILES: c1ccc(-c2ccc3c(c2)c2cc(-c4ccc5c(c4)c4ccccc4n5-c4ccccc4)ccc2n3-c2cccc(-c3cccc(-n4c5ccc(-c6ccccc6)cc5c5c(-c6cccc(-c7nc(-c8ccccc8)nc(-c8ccccc8)n7)c6)cccc54)c3)c2)cc1 ; c1ccc(-c2ccc3c(c2)c2cc(-c4ccc5c(c4)c4ccccc4n5-c4ccccc4)ccc2n3-c2ccc3c4ccccc4c4ccc(-n5c6ccc(-c7ccccc7)cc6c6c(-c7cccc(-c8nc(-c9ccccc9)nc(-c9ccccc9)n8)c7)cccc65)cc4c3c2)cc1 ; c1ccc(-c2ccc3c(c2)c2cc(-c4ccc5c(c4)c4ccccc4n5-c4ccccc4)ccc2n3-c2cc(-n3c4ccc(-c5ccccc5)cc4c4c(-c5cccc(-c6nc(-c7ccccc7)nc(-c7ccccc7)n6)c5)cccc43)cc3c2oc2ccccc23)cc1]

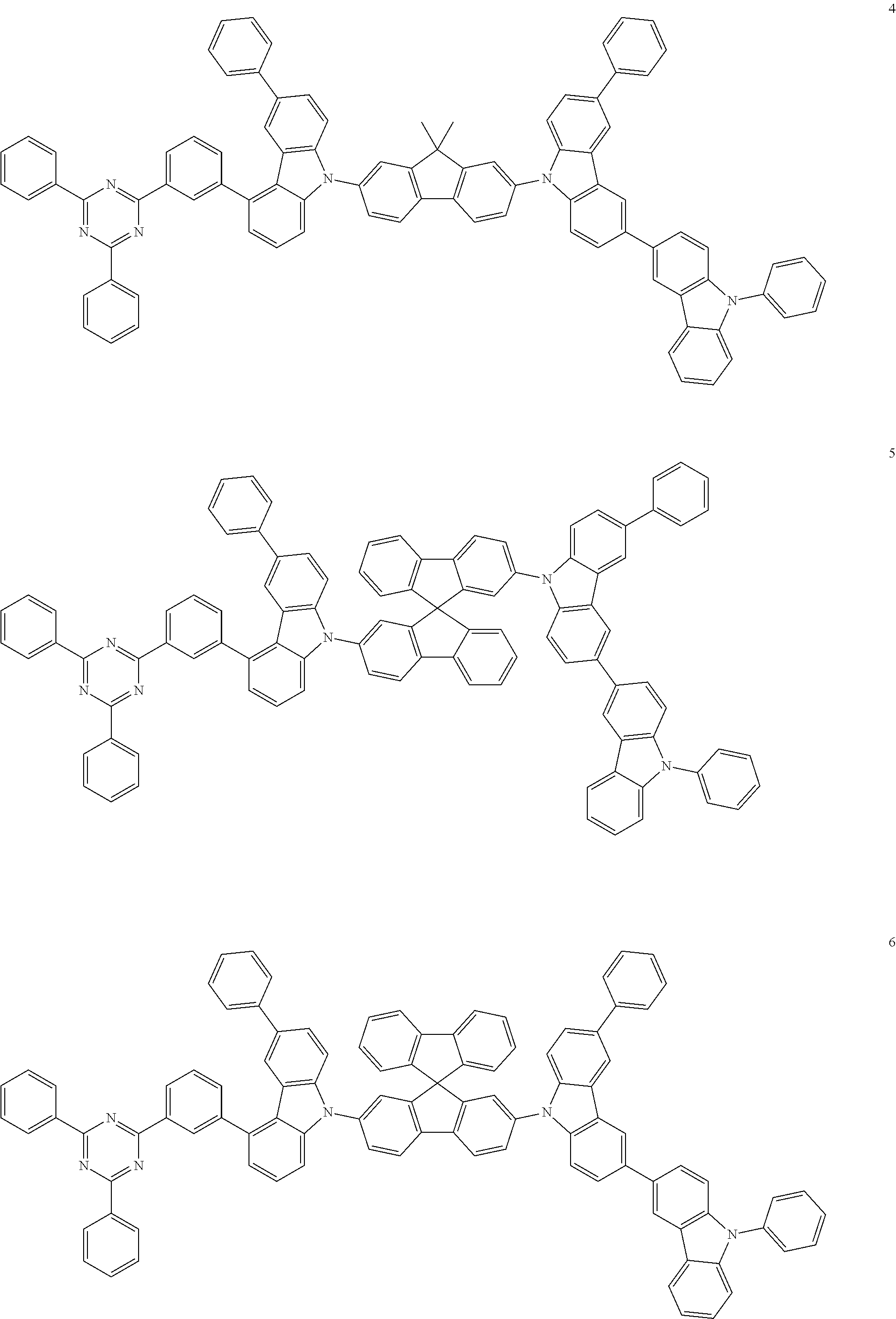

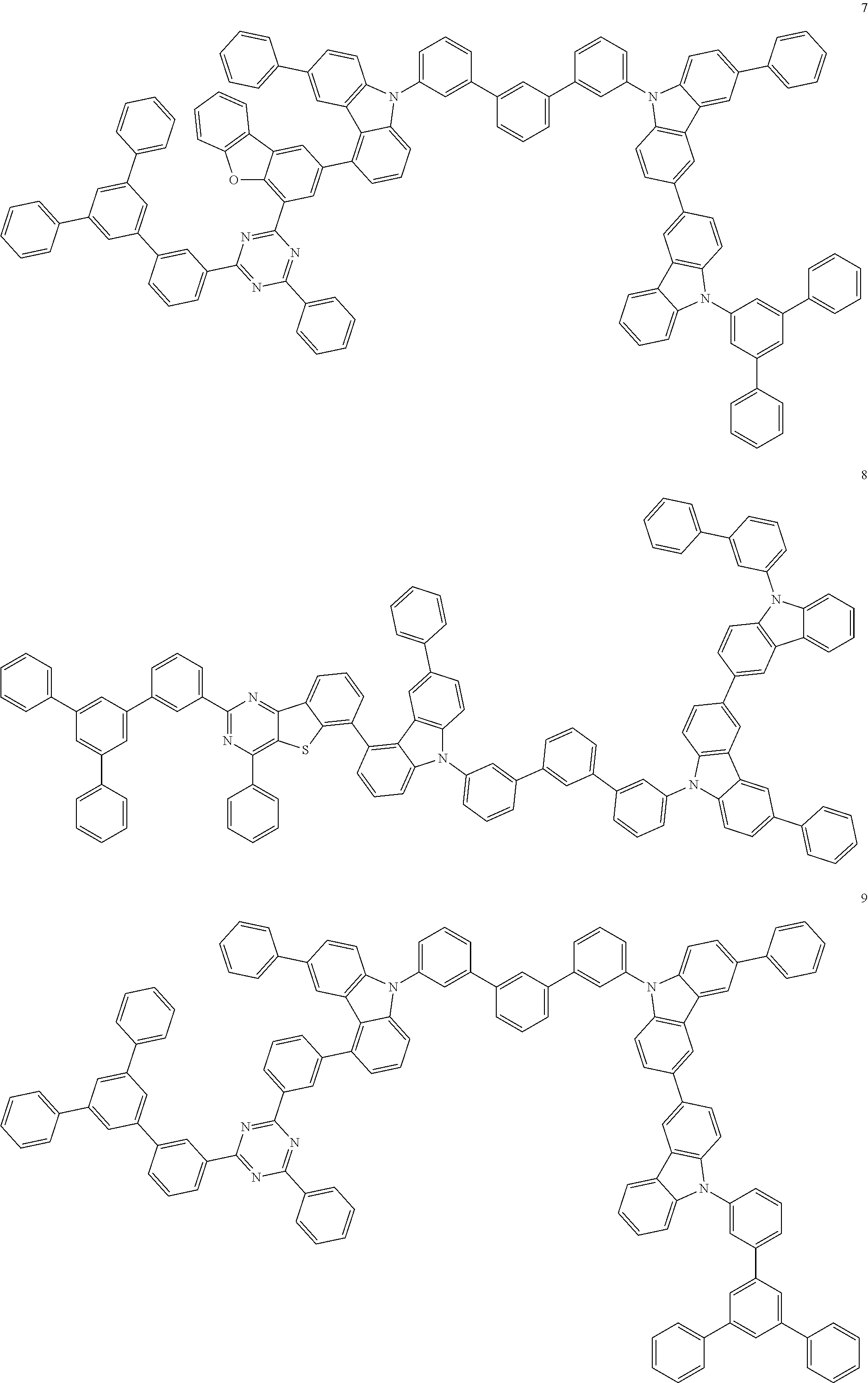

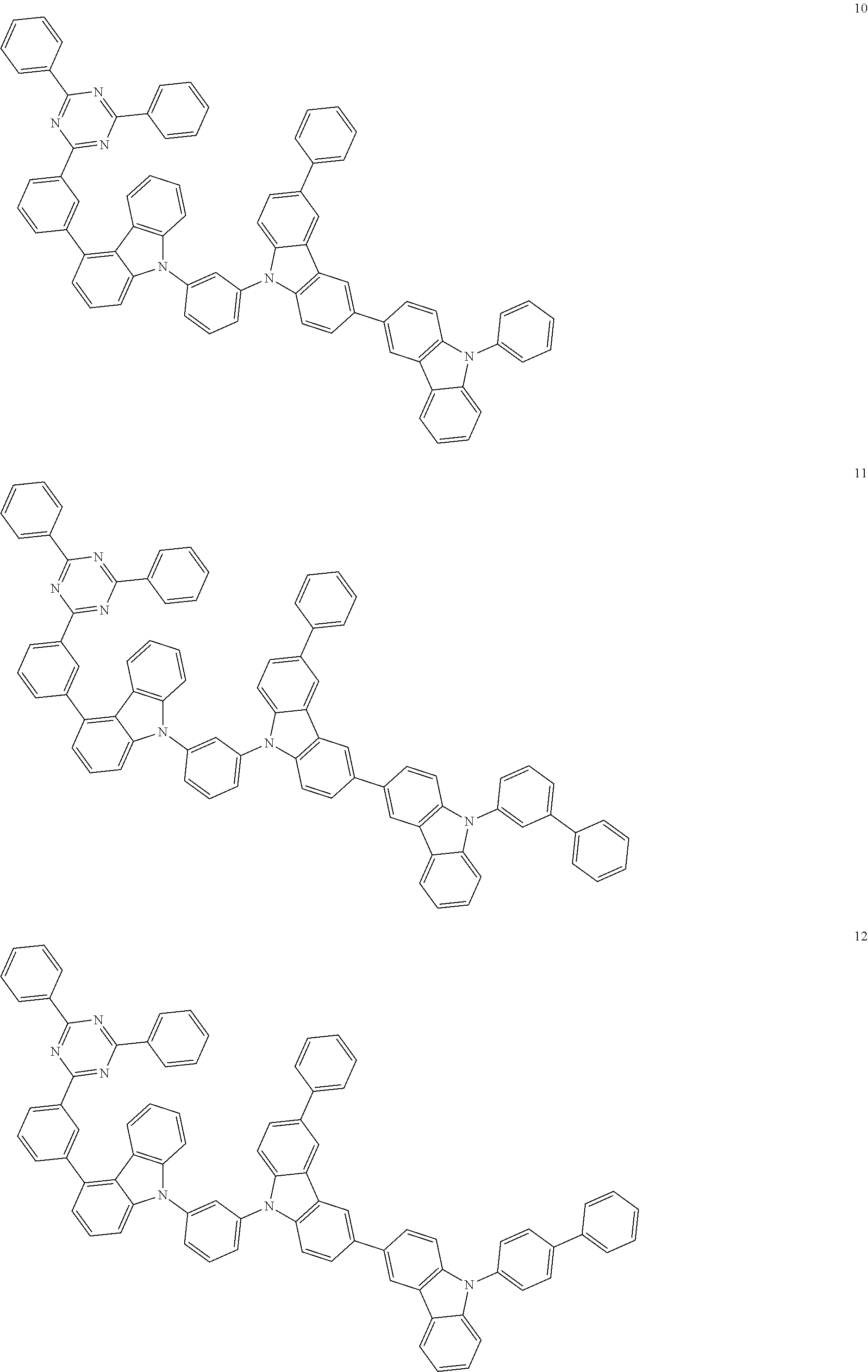 [SMILES: c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6nc(-c7ccccc7)nc(-c7ccccc7)n6)c5)cccc43)c2)cc1 ; c1ccc(-c2cccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)c2)cc1 ; c1ccc(-c2ccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)cc2)cc1]

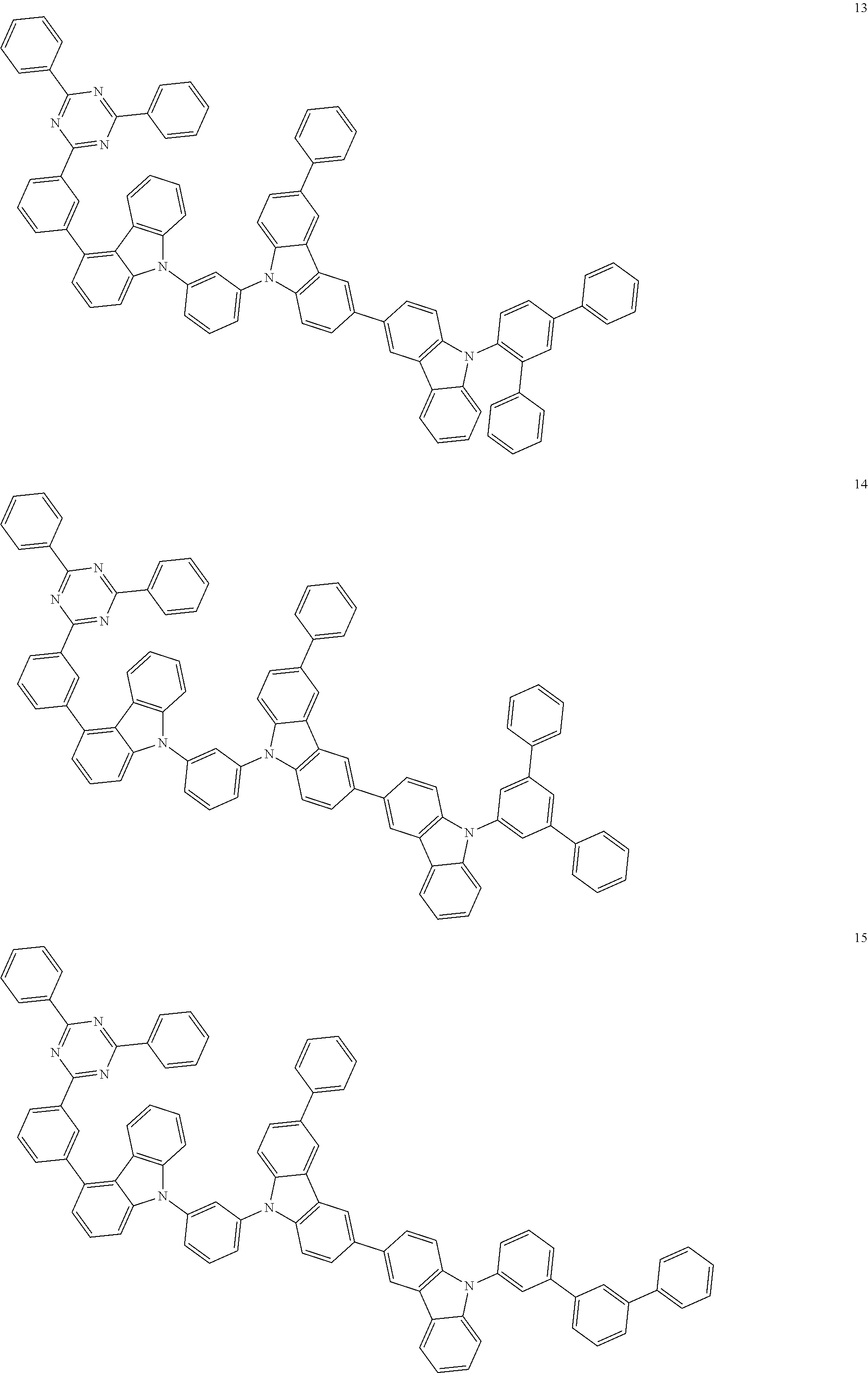

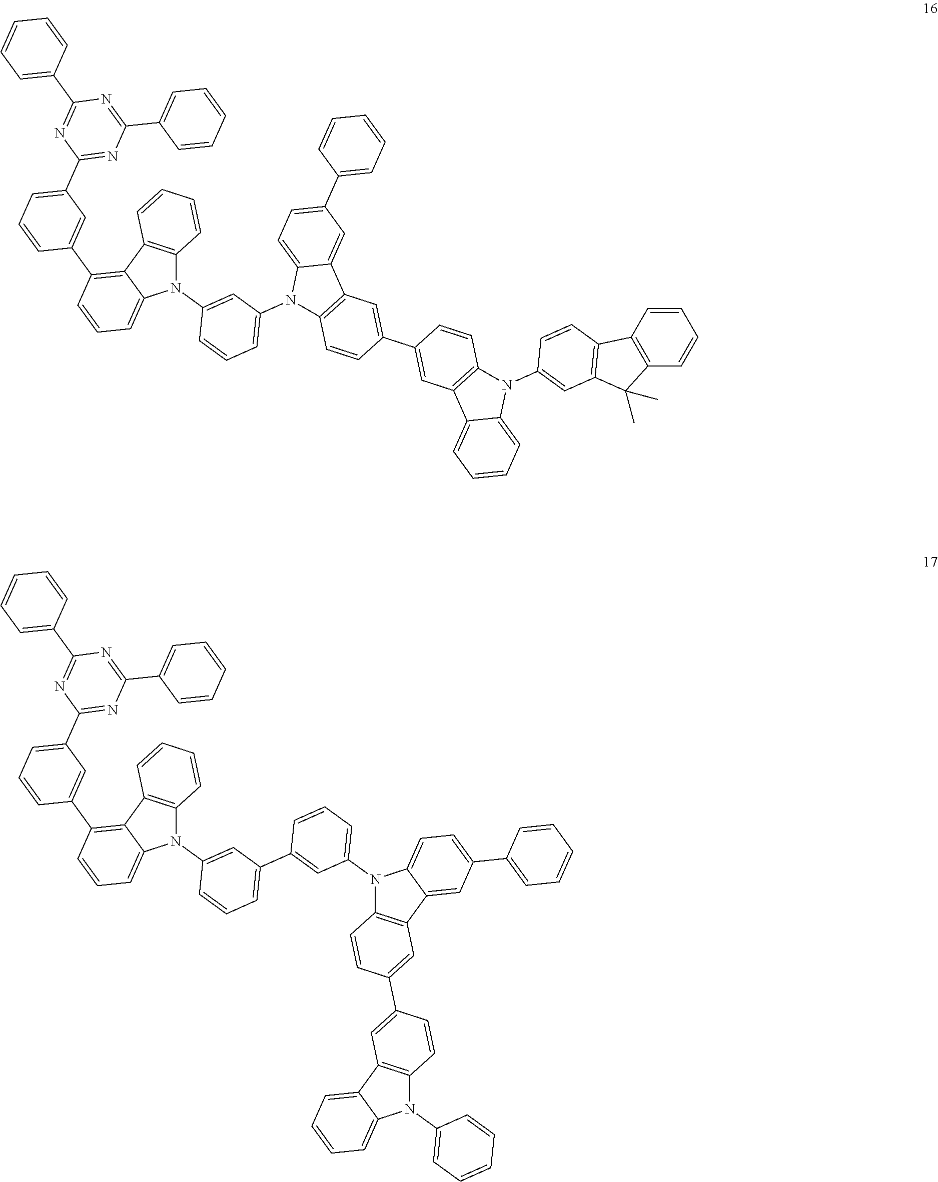 [SMILES: CC1(C)c2ccccc2-c2ccc(-n3c4ccccc4c4cc(-c5ccc6c(c5)c5cc(-c7ccccc7)ccc5n6-c5cccc(-n6c7ccccc7c7c(-c8cccc(-c9nc(-c%10ccccc%10)nc(-c%10ccccc%10)n9)c8)cccc76)c5)ccc43)cc21 ; c1ccc(-c2ccc3c(c2)c2cc(-c4ccc5c(c4)c4ccccc4n5-c4ccccc4)ccc2n3-c2cccc(-c3cccc(-n4c5ccccc5c5c(-c6cccc(-c7nc(-c8ccccc8)nc(-c8ccccc8)n7)c6)cccc54)c3)c2)cc1]

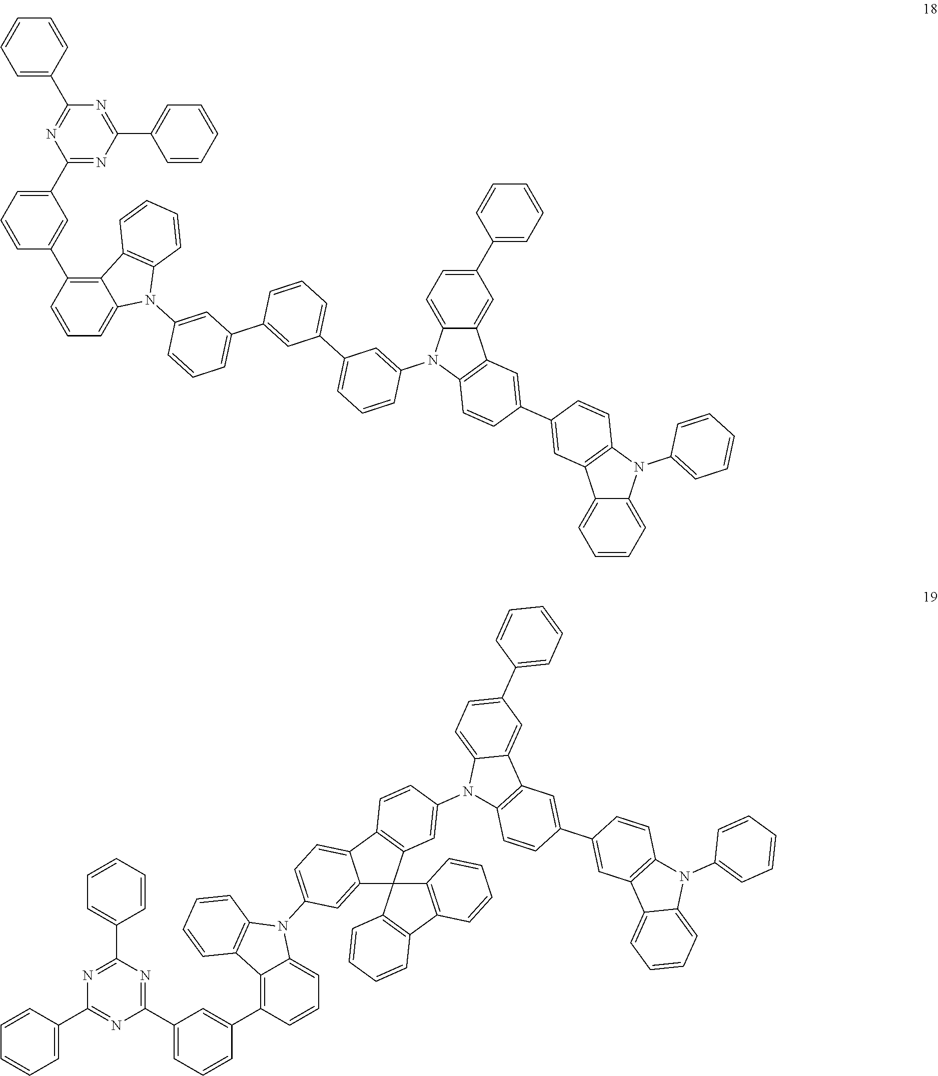 [SMILES: c1ccc(-c2ccc3c(c2)c2cc(-c4ccc5c(c4)c4ccccc4n5-c4ccccc4)ccc2n3-c2cccc(-c3cccc(-c4cccc(-n5c6ccccc6c6c(-c7cccc(-c8nc(-c9ccccc9)nc(-c9ccccc9)n8)c7)cccc65)c4)c3)c2)cc1 ; c1ccc(-c2ccc3c(c2)c2cc(-c4ccc5c(c4)c4ccccc4n5-c4ccccc4)ccc2n3-c2ccc3c(c2)C2(c4ccccc4-c4ccccc42)c2cc(-n4c5ccccc5c5c(-c6cccc(-c7nc(-c8ccccc8)nc(-c8ccccc8)n7)c6)cccc54)ccc2-3)cc1]

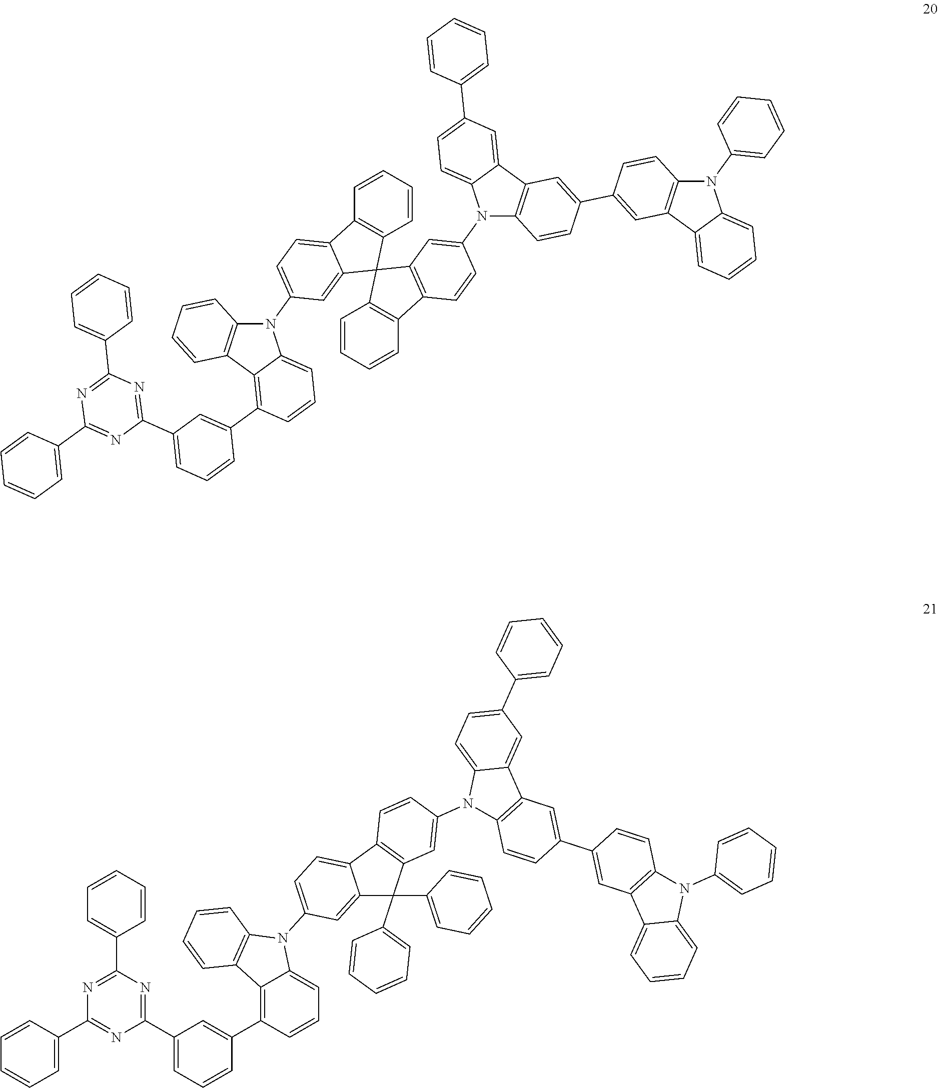 [SMILES: c1ccc(-c2ccc3c(c2)c2cc(-c4ccc5c(c4)c4ccccc4n5-c4ccccc4)ccc2n3-c2ccc3c(c2)C2(c4ccccc4-3)c3ccccc3-c3ccc(-n4c5ccccc5c5c(-c6cccc(-c7nc(-c8ccccc8)nc(-c8ccccc8)n7)c6)cccc54)cc32)cc1 ; c1ccc(-c2ccc3c(c2)c2cc(-c4ccc5c(c4)c4ccccc4n5-c4ccccc4)ccc2n3-c2ccc3c(c2)C(c2ccccc2)(c2ccccc2)c2cc(-n4c5ccccc5c5c(-c6cccc(-c7nc(-c8ccccc8)nc(-c8ccccc8)n7)c6)cccc54)ccc2-3)cc1]

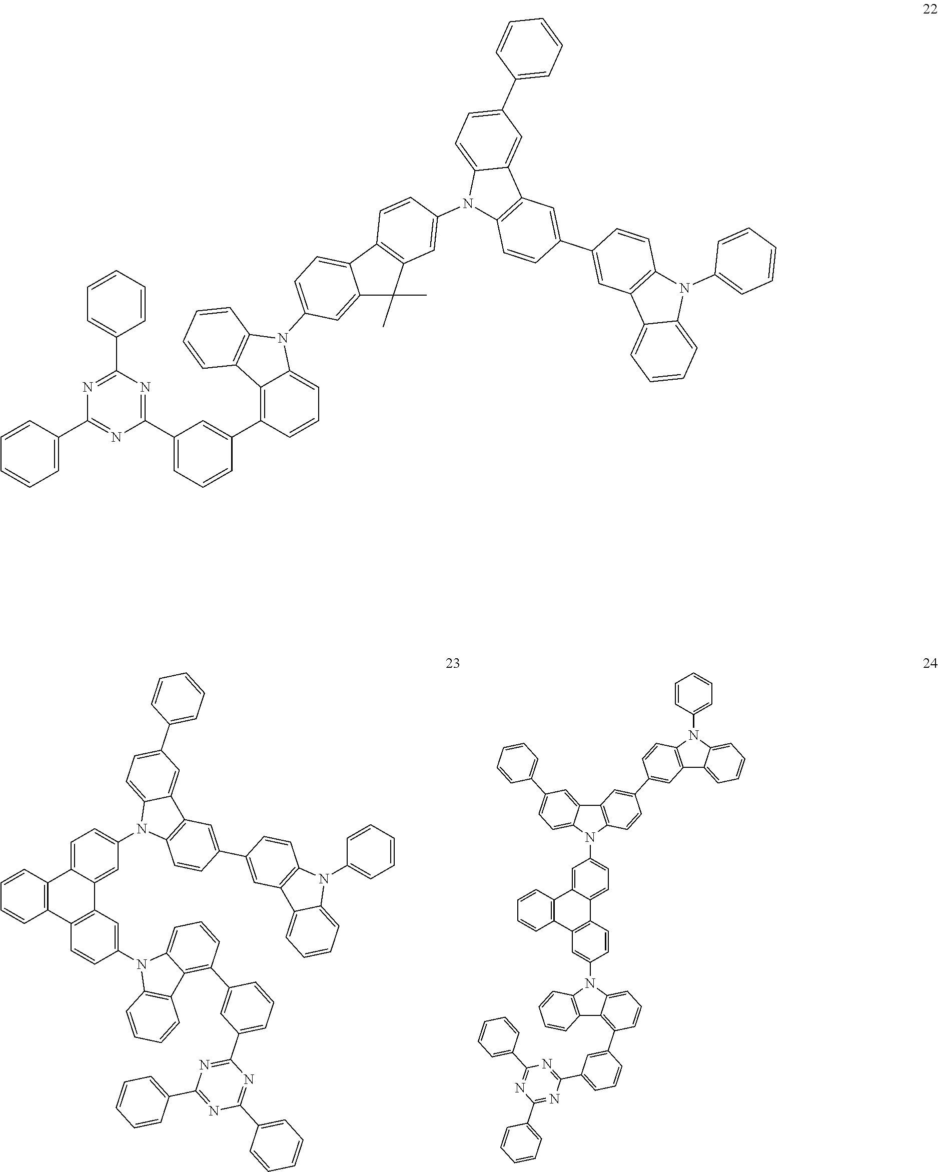 [SMILES: CC1(C)c2cc(-n3c4ccc(-c5ccccc5)cc4c4cc(-c5ccc6c(c5)c5ccccc5n6-c5ccccc5)ccc43)ccc2-c2ccc(-n3c4ccccc4c4c(-c5cccc(-c6nc(-c7ccccc7)nc(-c7ccccc7)n6)c5)cccc43)cc21 ; c1ccc(-c2ccc3c(c2)c2cc(-c4ccc5c(c4)c4ccccc4n5-c4ccccc4)ccc2n3-c2ccc3c4ccccc4c4ccc(-n5c6ccccc6c6c(-c7cccc(-c8nc(-c9ccccc9)nc(-c9ccccc9)n8)c7)cccc65)cc4c3c2)cc1 ; c1ccc(-c2ccc3c(c2)c2cc(-c4ccc5c(c4)c4ccccc4n5-c4ccccc4)ccc2n3-c2ccc3c4ccc(-n5c6ccccc6c6c(-c7cccc(-c8nc(-c9ccccc9)nc(-c9ccccc9)n8)c7)cccc65)cc4c4ccccc4c3c2)cc1]

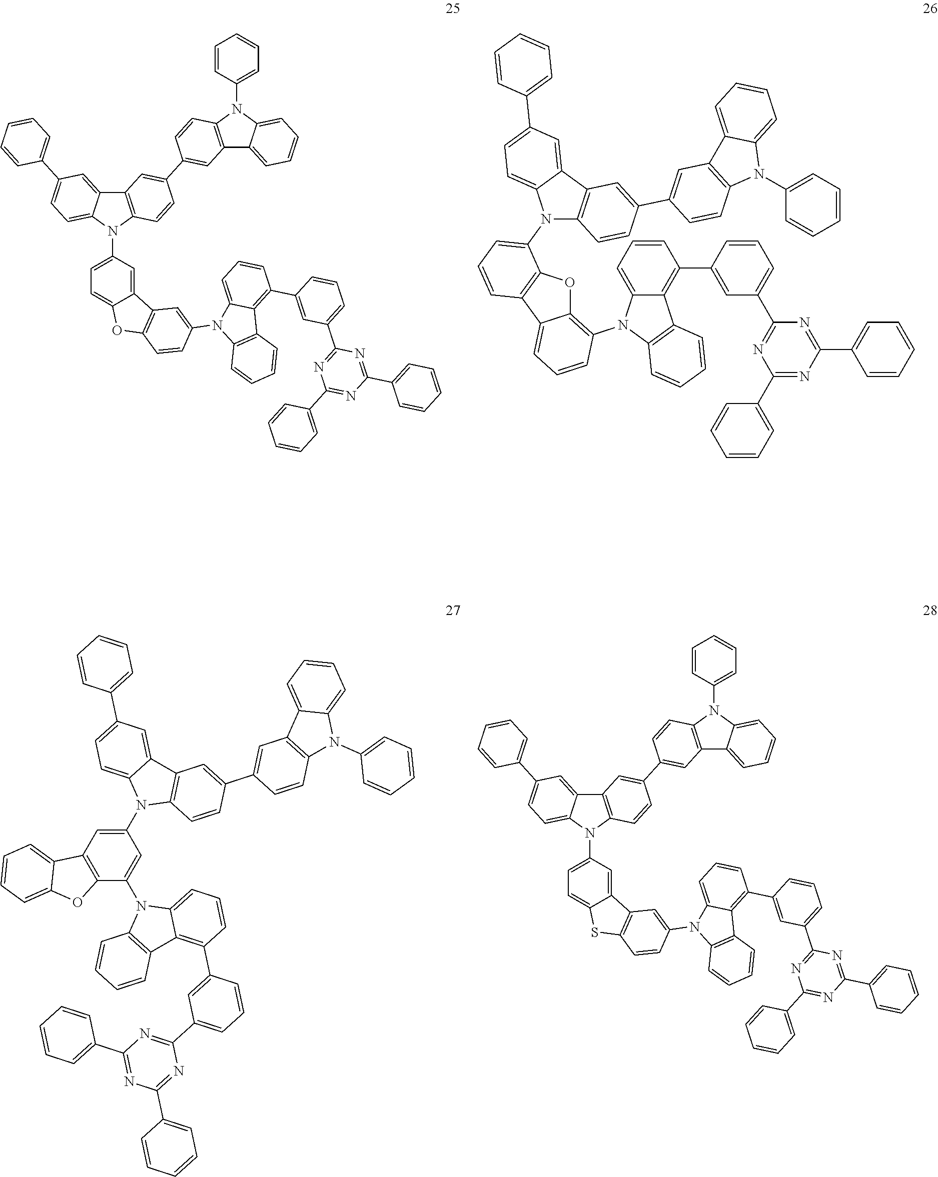

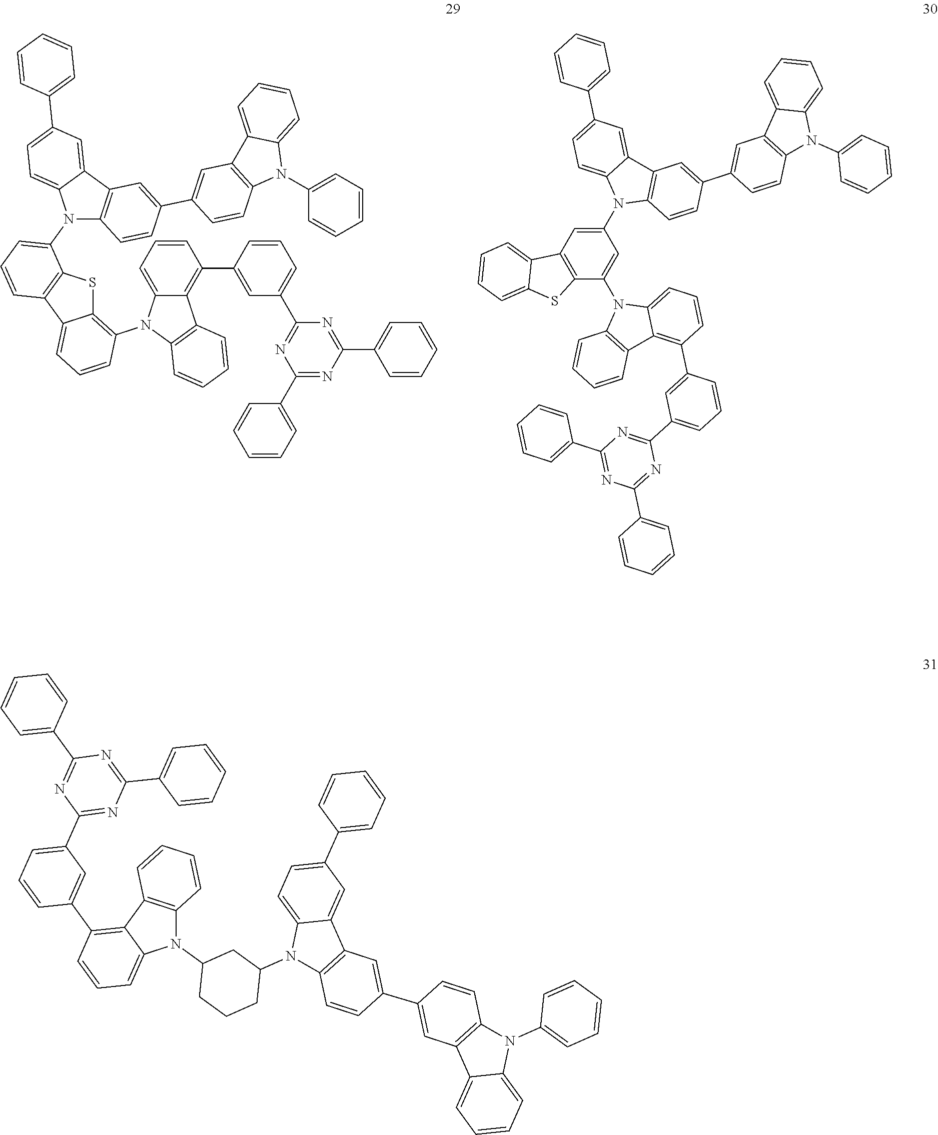 [SMILES: c1ccc(-c2ccc3c(c2)c2cc(-c4ccc5c(c4)c4ccccc4n5-c4ccccc4)ccc2n3-c2cccc3c2sc2c(-n4c5ccccc5c5c(-c6cccc(-c7nc(-c8ccccc8)nc(-c8ccccc8)n7)c6)cccc54)cccc23)cc1 ; c1ccc(-c2ccc3c(c2)c2cc(-c4ccc5c(c4)c4ccccc4n5-c4ccccc4)ccc2n3-c2cc(-n3c4ccccc4c4c(-c5cccc(-c6nc(-c7ccccc7)nc(-c7ccccc7)n6)c5)cccc43)c3sc4ccccc4c3c2)cc1 ; c1ccc(-c2ccc3c(c2)c2cc(-c4ccc5c(c4)c4ccccc4n5-c4ccccc4)ccc2n3C2CCCC(n3c4ccccc4c4c(-c5cccc(-c6nc(-c7ccccc7)nc(-c7ccccc7)n6)c5)cccc43)C2)cc1]

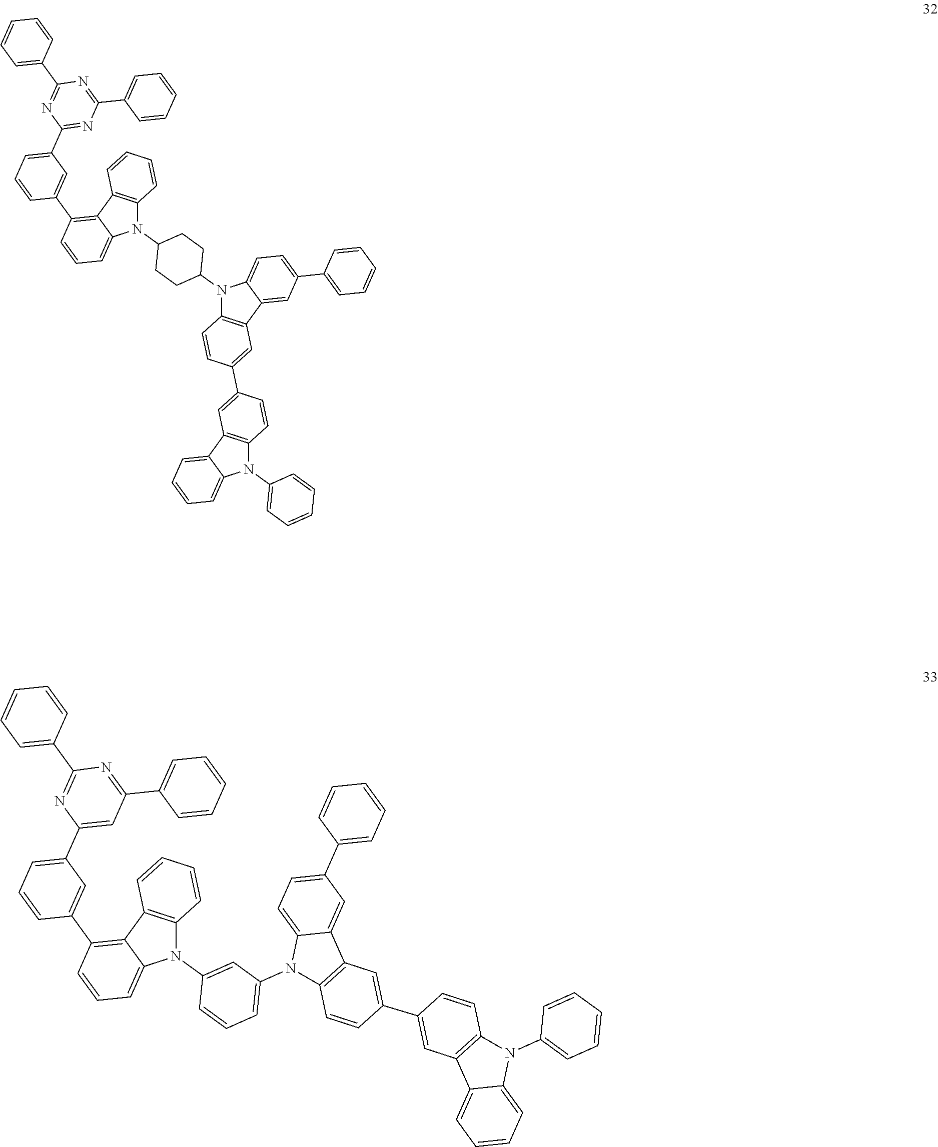 [SMILES: c1ccc(-c2ccc3c(c2)c2cc(-c4ccc5c(c4)c4ccccc4n5-c4ccccc4)ccc2n3C2CCC(n3c4ccccc4c4c(-c5cccc(-c6nc(-c7ccccc7)nc(-c7ccccc7)n6)c5)cccc43)CC2)cc1 ; c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6cc(-c7ccccc7)nc(-c7ccccc7)n6)c5)cccc43)c2)cc1]

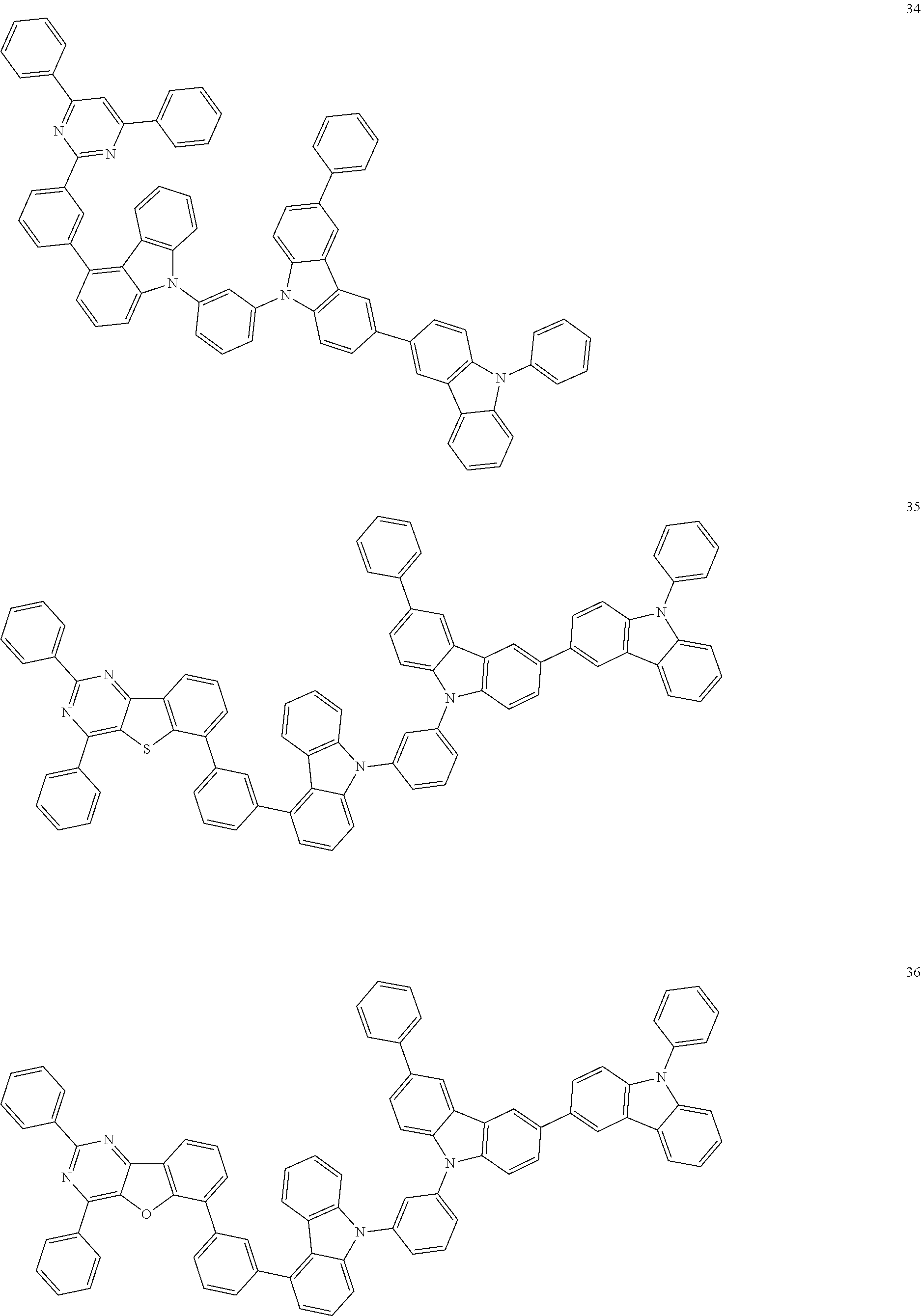 [SMILES: c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6nc(-c7ccccc7)cc(-c7ccccc7)n6)c5)cccc43)c2)cc1 ; c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6cccc7c6sc6c(-c8ccccc8)nc(-c8ccccc8)nc67)c5)cccc43)c2)cc1 ; c1ccc(-c2ccc3c(c2)c2cc(-c4ccc5c(c4)c4ccccc4n5-c4ccccc4)ccc2n3-c2cccc(-n3c4ccccc4c4c(-c5cccc(-c6cccc7c6oc6c(-c8ccccc8)nc(-c8ccccc8)nc67)c5)cccc43)c2)cc1]

14. A composition for an organic light-emitting device comprising at least one selected from the heterocyclic compound of claim 1.

15. The composition of claim 14, wherein the composition for an organic light-emitting device further comprises a solvent, and wherein a content of the heterocyclic compound in the composition for an organic light-emitting device is 0.01 percent by mass or greater.

16. An organic light-emitting device comprising:
a first electrode;
a second electrode; and
an organic layer disposed between the first electrode and the second electrode,
wherein the organic layer comprises an emission layer and at least one heterocyclic compound of claim 1.

17. The organic light-emitting device of claim 16, wherein
the first electrode is an anode,
the second electrode is a cathode,
the organic layer comprises a hole transport region disposed between the first electrode and the emission layer and an electron transport region disposed between the emission layer and the second electrode,
wherein the hole transport region comprises at least one selected from a hole injection layer, a hole transport layer, a buffer layer, an emission auxiliary layer, and an electron blocking layer, and
wherein the emission layer comprises the heterocyclic compound.

18. The organic light-emitting device of claim 17, wherein the heterocyclic compound emits light from triplet excitons.

19. The organic light-emitting device of claim 17, wherein the hole transport region comprises an amine group-containing polymer.

20. The organic light-emitting device of claim 19, wherein the hole transport region comprises a hole transport layer that is in direct contact with the emission layer, and wherein the hole transport layer comprises the amine group-containing polymer.
